(19) United States
(12) Patent Application Publication
(10) Pub. No.: US 2021/0147857 A1
(43) Pub. Date: May 20, 2021

(54) VECTORS & METHODS

(71) Applicant: SNIPR Technologies Limited, London (GB)

(72) Inventor: Jasper CLUBE, London (GB)

(21) Appl. No.: 17/103,812

(22) Filed: Nov. 24, 2020

Related U.S. Application Data
(63) Continuation of application No. 16/626,057, filed on Dec. 23, 2019.

(30) Foreign Application Priority Data
Jun. 25, 2017 (GB) 1710126.2

(51) Int. Cl. C12N 15/70; C12N 15/11; C12N 15/90

(52) U.S. Cl. CPC C12N 15/70; C12N 15/11; C12N 2310/20; C12N 2800/80; C12N 15/902

(57) ABSTRACT

The invention relates to vectors and methods for de-repressing Cas systems in host cells.

The invention relates to vectors and methods for de-repressing Cas or Cascade in host cells.

### BACKGROUND

The type I-E CRISPR-Cas system from Escherichia coli encodes six Cas genes in two operons (casABCDE and cas3) required for CRISPR RNA processing and the cleavage and degradation of target DNA. The casABCDE operon is repressed by H-NS in E. coli strains such as K-12.

Gomaa et al used a system consisting of two plasmids (pCasA-E and pCas3) that inducibly express all six E coli Cas genes. In addition, Gomaa et al generated a third plasmid, encoding an altered version of the endogenous CRISPR1 array in E. coli K-12 that accommodates the insertion of engineered spacer sequences. pCRISPR plasmids encoding engineered, genome-targeting spacers were transformed into E. coli K-12 substrain BW25113 cells that were pre-engineered with inducible expression of the T7 polymerase (BW25113-T7) and the two Cas-expressing plasmids (pCasA-E and pCas3). In an alternative, the authors forced expression of the chromosomally encoded Cas genes through deletion of the has gene.

Citorik et al discloses the use of conjugative plasmids or phage for delivery of nucleotide sequences encoding exogenous Streptococcus pyogenes Cas9 and a CRISPR locus into E. coli.

US20160333348 (SNIPR Technologies Limited) discloses the harnessing of active, endogenous Cas nuclease in bacteria, especially for targeting a species in a mixed bacterial population.

Whilst the state of the art, such as Gomaa et al, addresses the issue of repressed endogenous Cas by introducing an exogenous CRISPR/Cas system, this requires the construction of multiple vectors to accommodate all of the sequences encoding components such as CRISPR arrays, Cas3, CasA, B, C, D and E. The large amount of exogenous sequence that needs to be introduced thus requires many different vectors to enter the target bacterial cell, which reduces the probability of success and reduces the efficiency of the process. For example, targeting of natural human, animal, plant or environmental microbiomes does not allow for pre-manipulation of the target host cells to equip them with exogenous sequences encoding one or multiple components of an exogenous CRISPR/Cas system. Additionally, it would be preferable to reduce the number of nucleic acid vectors—preferably down to one—that need to enter the target cells to effect CRISPR/Cas activity (eg, killing) in the host cells.

The most commonly employed Cas9, measuring in at 4.2 kilobases (kb), comes from S pyogenes. The molecule's length pushes the limit of how much genetic material a vector (such as a bacteriophage) can accommodate, creating a barrier to using CRISPR in various settings (see Ran et al). S thermophilus Cas9 (UniProtKB—G3ECR1 (CAS9_STRTR)) nucleotide sequence has a size of 1.4 kb.

Solutions such as those employing active, endogenous Cas nucleases (eg, as described in US20160333348) are generally useful where this is a naturally active Cas in the host. These solutions avoid the need to use bulky exogenous Cas sequences, but it would be desirable to enable harnessing of endogenous Cas that is naturally repressed in the host, thereby addressing other naturally-occurring bacterial species. It is also desirable to be able to do this in wild-type cells, eg, bacterial or archaeal cells, such as for addressing microbiomes naturally found in humans, animals or environments.

### REFERENCES

Gomaa et al; MBio. 2014 Jan. 28; 5(1):e00928-13. doi: 10.1128/mBio.00928-13, “Programmable removal of bacterial strains by use of genome-targeting CRISPR-Cas systems”;
Citorik et al, Nat Biotechnol. 2014 November; 32(11):1141-5. doi: 10.1038/nbt.3011. Epub 2014 Sep. 21, “Sequence-specific antimicrobials using efficiently delivered RNA-guided nucleases.

### SUMMARY OF THE INVENTION

To this end, the invention provides:—

In a First Configuration

A nucleic acid vector for introduction into a host cell, wherein the cell comprises a CRISPR/Cas system that is repressed by a repressor in the cell, the vector comprising
(a) A nucleotide sequence encoding a de-repressor that is capable of de-repressing the CRISPR/Cas system in the cell, wherein the sequence is expressible in the cell to produce the de-repressor; and
(b) Optionally a CRISPR array for production of one or more crRNAs in the cell; and/or one or more nucleotide sequences each encoding a respective guide RNA (gRNA, eg, a single guide RNA) in the cell; wherein each crRNA or gRNA is capable of guiding Cas to modify a respective protospacer sequence of the host cell genome or to modify a protospacer sequence of an episome comprised by the host in the presence of the de-repressor.

In a Second Configuration

A nucleic acid vector for introduction into a bacterial or archaeal host cell, wherein the cell comprises an endogenous CRISPR/Cas system that is naturally repressed by a repressor in the cell, the vector comprising
(a) A nucleotide sequence encoding a de-repressor that is capable of de-repressing the CRISPR/Cas system in the cell, wherein the sequence is expressible in the cell to produce the de-repressor; and
(b) Optionally a CRISPR array for production of one or more crRNAs in the cell; and/or one or more nucleotide sequences each encoding a respective guide RNA (gRNA, eg, a single guide RNA) in the cell; wherein each crRNA or gRNA is capable of guiding Cas to modify a protospacer sequence of the host cell genome or to modify a protospacer sequence of an episome comprised by the host in the presence of the de-repressor;
   Wherein the repressor is H-NS, StpA, LRP or CRP or a homologue, or orthologue or functional equivalent thereof encoded by the cell genome; and
   Wherein the de-repressor is LeuO or a homologue, or orthologue or functional equivalent thereof that is capable of forming a complex with H-NS; or a mutant of H-NS, StpA, LRP or CRP that is capable of forming a complex with H-NS, StpA, LRP or CRP repressor respectively.

In a Third Configuration

A medicament comprising a plurality of vectors according to any preceding configuration, optionally further comprising one or more medical drugs (eg, an anti-cancer medicament) or antibiotics (eg, wherein the protospacer sequence is comprised by a host cell antibiotic resistance gene), for treating or preventing a disease or condition in a human or animal.

In a Fourth Configuration

A method of treating or preventing a disease or condition in a human or animal subject, the method comprising administering a vector or medicament of any preceding configuration to the subject, wherein host cells comprised by a microbiome of the subject are modified by endogenous de-repressed Cas of the cells, and the treatment or prevention is carried out.

In a Fifth Configuration

A method of killing a wild-type bacterial or archaeal cell (eg, E coli or Salmonella cell), wherein the cell comprises an endogenous CRISPR/Cas system comprising nucleotide sequences encoding Cas3 and Cascade proteins, wherein Cas3 and/or Cascade is naturally repressed in the cell, the method comprising
(a) de-repressing said Cas3 and/or Cascade and
(b) introducing into the cell (i) a CRISPR array for production of one or more crRNAs in the cell; or (ii) one or more nucleotide sequences each encoding a respective guide RNA (gRNA, eg, a single guide RNA);
   wherein each crRNA or gRNA guides Cas or Cascade to modify a respective protospacer sequence of the host cell genome or to modify a protospacer sequence of an episome comprised by the host.

In a Sixth Configuration

A nucleic acid vector for introduction into a host cell, wherein the cell comprises a CRISPR/Cas system that is repressed by a repressor in the cell, the vector comprising
(a) A nucleotide sequence encoding a de-repressor that is capable of de-repressing the CRISPR/Cas system in the cell, wherein the sequence is expressible in the cell to produce the de-repressor; and
(b) A site for introduction of
   (i) a CRISPR array or a CRISPR spacer sequence for production of one or more crRNAs in the cell; or
   (ii) a nucleotide sequence encoding a guide RNA (gRNA, eg, a single guide RNA) in the cell;
      wherein said crRNA or gRNA is capable of guiding Cas to modify a respective protospacer sequence of the host cell genome or to modify a protospacer sequence of an episome comprised by the host in the presence of the de-repressor.

### BRIEF DESCRIPTION OF THE FIGURES

FIG. 1 shows regulators controlling the expression of spCas9 and the self-targeting sgRNA targeting the ribosomal RNA subunit 16s.

FIG. 2 shows specific targeting of E. coli strain by an exogenous CRISPR-Cas system. The sgRNA target the genome of K-12 derived E. coli strains, like E. coli TOP10, while the other strain tested was unaffected.

FIG. 3 shows spot assay with serial dilutions of individual bacterial species used in this study and mixed culture in TH agar without induction of CRISPR-Cas9 system.

FIG. 4 shows spot assay of the dilution 10³ on different selective media. TH with 2.5 g 1⁻¹ PEA is a selective media for B. subtilis alone. MacConkey supplemented with maltose is a selective and differential culture medium for bacteria designed to selectively isolate Gram-negative and enteric bacilli and differentiate them based on maltose fermentation. Therefore TOP10 ΔmalK mutant makes white colonies on the plates while Nissle makes pink colonies; A is E coli ΔmalK, B is E coli Nissile, C is B subtilis, D is L lactis, E is mixed culture; the images at MacConkey-/B and E appear pink; the images at MacConkey+/B and E appear pink.

FIG. 5 shows selective growth of the bacteria used in this study on different media and selective plates.

### DETAILED DESCRIPTION

The invention provides, inter alia, vectors, compositions comprising a plurality of said vectors, methods and uses. The invention is useful for targeting wild-type bacterial populations found naturally in the environment (eg, in water or waterways, cooling or heating equipment, in or on agricultural plants, in soil), comprised by beverages and foodstuffs (or equipment for manufacturing, processing or storing these) or wild-type bacterial populations comprised by human or animal microbiota (eg, in the gut, lungs or on the skin). Thus, the invention finds utility in situations when pre-modification of host cells to make them receptive to killing or growth inhibition is not possible or desirable (eg, when treatment in situ of microbiota in the gut or other locations of a subject is desired). In another application, the invention finds utility for producing ex vivo a medicament (eg, a gut bacterial transplant) for administration to a human or animal subject for treating or preventing a disease or condition caused or mediated by the host cells, wherein the medicament comprises a modified mixed bacterial population (eg, obtained from faeces or gut microbiota of one or more human donors) which is the product of the use or method of the invention, wherein the population comprises a sub-population of bacteria of a species or strain that is different to the species or strain of the host cells. The former sub-population cells do not comprise the protospacer target and thus are not modified by the use or method. Thus, for example, the method can be used to reduce the proportion of a specific sub-population and spare Bacteroidetes in the mixed population, eg, for producing a medicament for treating or preventing a metabolic or GI condition (eg, colitis) or disease disclosed herein. In this way, the invention can provide a modified bacterial transplant (eg, a modified faecal transplant) medicament for such use or for said treatment or prevention in a human or animal. For example, the method can be used to modify one or more microbiota in vitro to produce a modified collection of bacteria for administration to a human or animal for medical use (eg, treatment or prevention of a metabolic condition (such as obesity or diabetes) or a GI tract condition (eg, colitis, IBD, IBS, Crohn's disease or any such condition mentioned herein) or a cancer (eg, a GI tract cancer or melanoma)) or for cosmetic or personal hygiene use (eg, for topical use on a human, eg, for reducing armpit or other body odour by topical application to an armpit of a human or other relevant location of a human). In another example, vectors of the invention are administered to a human or animal and the host cells are harboured by the human or animal, eg, comprised by a microbiota of the human or animal (such as a gut microbiota or any other type of microbiota disclosed herein). In this way, a disease or condition mediated or caused by the host cells can be treated or prevented. In an example, host cell transformation is carried out in vitro and optionally the vectors are plasmids or phagemids that are electroporated into host cells; alternatively the vectors are comprised by viruses (eg, bacteriophage) that infect the host cells and introduce the de-repressor and array or gRNA-encoding sequences into the host cells. In an example, the nucleic acid are RNA (eg, copies of the gRNA). In another example, the vectors are DNA vectors or RNA vectors.

In an example, the organism is a plant or animal, eg, vertebrate (eg, any mammal or human disclosed herein) or crop or food plant.

In an example, the method, use, vector or composition is for medical or dental or opthalmic use (eg, for treating or preventing an infection in an organism or limiting spread of the infection in an organism).

In an example, the method, use, vector or composition is for cosmetic use (eg, use in a cosmetic product, eg, make-up), or for hygiene use (eg, use in a hygiene product, eg, soap).

In an example, the composition is as any of the following: In an example, the composition is a medical, opthalmic, dental or pharmaceutical composition (eg, comprised by a an anti-host vaccine). In an example, the composition is a an antimicrobial composition, eg, an antibiotic or antiviral, eg, a medicine, disinfectant or mouthwash. In an example, the composition is a cosmetic composition (eg, face or body make-up composition). In an example, the composition is a herbicide. In an example, the composition is a pesticide (eg, when the host is a Bacillus (eg, thuringiensis) host). In an example, the composition is a beverage (eg, beer, wine or alcoholic beverage) additive. In an example, the composition is a food additive (eg, where the host is an E coli, Salmonella, Listeria or Clostridium (eg, botulinum) host). In an example, the composition is a water additive. In an example, the composition is a additive for aquatic animal environments (eg, in a fish tank). In an example, the composition is an oil or petrochemical industry composition or comprised in such a composition (eg, when the host is a sulphate-reducing bacterium, eg, a Desulfovibrio host). In an example, the composition is a oil or petrochemical additive. In an example, the composition is a chemical additive. In an example, the composition is a disinfectant (eg, for sterilizing equipment for human or animal use, eg, for surgical or medical use, or for baby feeding). In an example, the composition is a personal hygiene composition for human or animal use. In an example, the composition is a composition for environmental use, eg, for soil treatment or environmental decontamination (eg, from sewage, or from oil, a petrochemical or a chemical, eg, when the host is a sulphate-reducing bacterium, eg, a Desulfovibrio host). In an example, the composition is a plant growth stimulator. In an example, the composition is a composition for use in oil, petrochemical, metal or mineral extraction. In an example, the composition is a fabric treatment or additive. In an example, the composition is an animal hide, leather or suede treatment or additive. In an example, the composition is a dye additive. In an example, the composition is a beverage (eg, beer or wine) brewing or fermentation additive (eg, when the host is a Lactobacillus host). In an example, the composition is a paper additive. In an example, the composition is an ink additive. In an example, the composition is a glue additive. In an example, the composition is an anti-human or animal or plant parasitic composition. In an example, the composition is an air additive (eg, for air in or produced by air conditioning equipment, eg, where the host is a Legionella host). In an example, the composition is an anti-freeze additive (eg, where the host is a Legionella host). In an example, the composition is an eyewash or opthalmic composition (eg, a contact lens fluid). In an example, the composition is comprised by a dairy food (eg, the composition is in or is a milk or milk product; eg, wherein the host is a Lactobacillus, Streptococcus, Lactococcus or Listeria host). In an example, the composition is or is comprised by a domestic or industrial cleaning product (eg, where the host is an E. coli, Salmonella, Listeria or Clostridium (eg, botulinum) host). In an example, the composition is comprised by a fuel. In an example, the composition is comprised by a solvent (eg, other than water). In an example, the composition is a baking additive (eg, a food baking additive). In an example, the composition is a laboratory reagent (eg, for use in biotechnology or recombinant DNA or RNA technology). In an example, the composition is comprised by a fibre retting agent. In an example, the composition is for use in a vitamin synthesis process. In an example, the composition is an anti-crop or plant spoiling composition (eg, when the host is a saprotrophic bacterium). In an example, the composition is an anti-corrosion compound, eg, for preventing or reducing metal corrosion (eg, when the host is a sulphate-reducing bacterium, eg, a Desulfovibrio host, eg for use in reducing or preventing corrosion of oil extraction, treatment or containment equipment; metal extraction, treatment or containment equipment; or mineral extraction, treatment or containment equipment). In an example, the composition is an agricultural or farming composition or comprised in such a composition. In an example, the composition is a silage additive. The invention provides a CRISPR array, gRNA-encoding nucleotide sequence, vector or plurality of vectors described herein for use in any of the compositions described in this paragraph or for use in any application described in this paragraph, eg, wherein the host cell is a bacterial or archaeal cell. The invention provides a method for any application described in this paragraph, wherein the method comprises combining a CRISPR array, gRNA-encoding nucleotide sequence, vector or plurality of the invention with a host cell (eg, bacterial or archaeal cell). In an embodiment, the host cell is not present in or on a human (or human embryo) or animal.

Any aspect of the present invention, eg, array, vector, composition, use or method, is for an industrial or domestic use, or is used in a method for such use. For example, it is for or used in agriculture, oil or petroleum industry, food or drink industry, clothing industry, packaging industry, electronics industry, computer industry, environmental industry, chemical industry, aerospace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.

The invention provides a nucleic acid vector for introduction into a host cell, wherein the cell comprises a CRISPR/Cas system that is repressed by a repressor in the cell, the vector comprising
(a) A nucleotide sequence encoding a de-repressor that is capable of de-repressing the CRISPR/Cas system in the cell, wherein the sequence is expressible in the cell to produce the de-repressor; and
(b) A CRISPR array for production of one or more crRNAs in the cell; and/or one or more nucleotide sequences each encoding a respective guide RNA (gRNA, eg, a single guide RNA) in the cell; wherein each crRNA or gRNA is capable of guiding Cas to modify a respective protospacer sequence of the host cell genome or to modify a protospacer sequence of an episome comprised by the host in the presence of the de-repressor.

The sequence of (a) is expressible as it may be operably connected to a promoter that is operable in the cell for expression of the de-repressor.

Optionally, any host cell(s) herein is/are bacterial or archaeal cells. In an example, the cell(s) is/are in stationary phase. In an example, the cell(s) is/are in exponential phase. In an example, the cell(s) is/are in lag phase. In an example, the cell(s) is/are wild-type cells or naturally-occurring cells, eg, comprised by a naturally-occurring microbiome, eg, of a human, animal, plant, soil, water, sea, waterway or environment. In an example, the cell(s) is/are artificially genetically modified. In an example, the CRISPR/Cas system is artificially repressed and the de-repressor removes or reduced said repression.

In an example, a plurality of vectors of the invention are introduced into a plurality of said host cells, wherein the host cells are comprised by a bacterial population, eg, ex vivo, in vivo or in vitro. In an example, the host cells are comprised by a microbiota population comprised by an organism or environment (eg, a waterway microbiota, water microbiota, human or animal gut microbiota, human or animal oral cavity microbiota, human or animal vaginal microbiota, human or animal skin or hair microbiota or human or animal armpit microbiota), the population comprising first bacteria that are symbiotic or commensal with the organism or environment and second bacteria comprising said host cells, wherein the host cells are detrimental (eg, pathogenic) to the organism or environment. In an embodiment, the population is ex vivo. In an example, the ratio of the first bacteria sub-population to the second bacteria sub-population is increased. In an example, the first bacteria are Bacteroides (eg, B. fragalis and/or B. thetaiotamicron) bacteria. Optionally, the Bacteroides comprises one, two, three or more Bacteroides species selected from caccae, capillosus, cellulosilyticus, coprocola, coprophilus, coprosuis, distasonis, dorei, eggerthii, faecis, finegoldii, fluxus, fragalis, intestinalis, melaninogenicus, nordii, oleiciplenus, oralis, ovatus, pectinophilus, plebeius, stercoris, thetaiotaomicron, uniformis, vulgatus and xylanisolvens. For example, the Bacteroides is or comprises B thetaiotaomicron. For example, the Bacteroides is or comprises B. fragalis.

In an example, the host, first or second cells are any bacterial species disclosed in US20160333348, GB1609811.3, PCT/EP2017/063593 and all US equivalent applications. The disclosures of these species (including specifically, Table 1 of PCT/EP2017/063593), are incorporated herein in their entirety and for potential inclusion of one or more disclosures therein in one or more claims herein.

In an example, the host cell(s) or bacterial population is harboured by a beverage or water (eg, a waterway or drinking water) for human consumption. In an example, the host cell(s) or said population is comprised by a composition (eg, a medicament (eg, bacterial gut transplant), beverage, mouthwash or foodstuff) for administration to a human or non-human animal for populating and rebalancing the gut or oral microbiota thereof (eg, wherein said use of the medicament is to treat or prevent a disease or condition in the human or animal) In an example, the host cell(s) or said population are on a solid surface or comprised by a biofilm (eg, a gut biofilm or a biofilm on an industrial apparatus). In an example of the vector, method or use is for in vitro treating an industrial or medical fluid, solid surface, apparatus or container (eg, for food, consumer goods, cosmetics, personal healthcare product, petroleum or oil production); or for treating a waterway, water, a beverage, a foodstuff or a cosmetic, wherein the host cell(s) are comprised by or on the fluid, surface, apparatus, container, waterway, water, beverage, foodstuff or cosmetic.

In an example, the invention provides a container for medical or nutritional use, wherein the container comprises a population or the product of the use or method. For example, the container is a sterilised container, eg, an inhaler or connected to a syringe or IV needle. In an example, the product population of the use or method is useful for administration to a human or animal to populate a microbiome thereof to treat or prevent a disease or condition (eg, a disease or condition recited herein) in the human or animal. The invention provides: A foodstuff or beverage for human or non-human animal consumption comprising the population product of the use or method of the invention.

In an example, the vector(s), composition is for administration (or is administered) to the human or non-human animal by mucosal, gut, oral, intranasal, intrarectal, intravaginal, ocular or buccal administration.

Optionally, the vector or vectors lack a Cas (eg, a Cas3 and/or Cas9) nuclease-encoding sequence. In an example, the system comprises repressed host cell Cascade, Cas3, CasCas9 or cpf1 activity.

In an example, the host cells are wild-type (eg, non-engineered) bacterial cells. In another example, the host cells are engineered (such as to introduce an exogenous nucleotide sequence chromosomally or to modify an endogenous nucleotide sequence, eg, on a chromosome or plasmid of the host cell). In an example, the formation of bacterial colonies of said host cells is inhibited following introduction of the vector(s) into the host cell(s). In an example, proliferation of host cells is inhibited following said introduction. In an example, host cell(s) are killed following said introduction.

Optionally, each host cell is of a strain or species found in human microbiota, optionally wherein the host cells are mixed with cells of a different strain or species, wherein the different cells are Enterobacteriaceae or bacteria that are probiotic, commensal or symbiotic with humans (eg, in the human gut. In an example, the host cell is an E coli or Salmonella cell.

The invention is optionally for inhibiting bacterial population growth or altering the relative ratio of sub-populations of first and second bacteria in a mixed population of bacteria, eg, for altering human or animal microbiomes, such as for the alteration of the proportion of Bacteroidetes (eg, Bacteroides, eg, fragalis and/or thetaiotamicron), Firmicutes and/or gram positive or negative bacteria in microbiota of a human. For example, an embodiment of the invention provides:—

An antimicrobial composition for use in a method of treating or preventing a disease in a human or animal subject, wherein the gut of the subject comprises a mixed bacterial population, the method comprising administering the antimicrobial to the subject to modify host cells of the mixed bacterial population comprised by the gut of the subject, to favour commensal or symbiotic Bacteroidetes of the gut population, thereby increasing the proportion of Bacteroidetes bacteria in the gut of the subject, wherein said treatment or prevention is effected, wherein the composition comprises one or more vectors of the invention and the host cells comprise a CRISPR/Cas system that is naturally repressed in the gut population.

Harnessing commensal and symbiotic Bacteroidetes of the subject is advantageous for exploiting disease-modifying effects by activity of the endogenous commensals and symbionts already in the gut. This avoids the risk of dosing with exogenous Bacteroidetes, which are potentially pathogenic as taught in the art. Furthermore, it may be possible to generate a more sustainable effect by exploiting the patient's own gut bacteria (eg, by creating niches in the patient gut microbiome for expansion by targeting other gut bacteria such as gram-positives, eg, Clostridium). The possibility for useful harnessing of endogenous patient bacteria also avoids the need for consideration and maintenance of dosing of formulations using bacterial preparations or extracts thereof.

Additionally, Bacteroides such as B. fragalis and B. thetaiotamicron are strict anaerobes, which severely limits production, storage and administration of compositions in anaerobic environments. The invention avoids that by harnessing the patient's own Bacteroidetes, which are retained in the compatible anaerobic environment of the gut.

Further, the patient's own endogenous Bacteroidetes and the patient's immune system and other interacting factors in the gut have evolved together and are matched to work effectively (eg, to stimulate useful immune responses for addressing disease), and the invention can exploit this advantage by harnessing the endogenous gut Bacteroidetes, eg, for stimulating an immune response in the subject. Additionally, it is not necessary for exogenously administered Bacteroidetes (or peptides thereof) which may be cleared somewhat (a dosing issue) and need somehow find their way to effectively colonise the correct intestinal crypt location for beneficial use. Instead the effector bacteria in the invention are already in position in the patient and are immediately useful.

In an example, the method is for treating or preventing an inflammatory bowel disease (IBD). In an example, the method is for treating or preventing obesity for medical purposes. In an example, the method is for treating or preventing diabetes. In an example, the method comprises increasing the relative ratio of Bacteroidetes versus Firmicutes. In an example, the Bacteroidetes are B. fragalis and/or B. thetaiotamicron. In an example, the Bacteroidetes are B. uniformis.

In an example, the vectors of the invention are for use in any method disclosed in US20160333348, GB1609811.3, PCT/EP2017/063593 and all US equivalent applications; in an example, the vectors of the invention are according to any vector disclosed in US20160333348, GB1609811.3, PCT/EP2017/063593 and all US equivalent applications. The disclosure of US20160333348, GB1609811.3, PCT/EP2017/063593 and all US equivalent applications, including these specific disclosures, are incorporated herein in its entirety and for potential inclusion of one or more disclosures therein in one or more claims herein.

In an example, the vector(s) or composition of the invention comprises a nucleotide sequence for expressing in the host cell an endolysin for host cell lysis, optionally wherein the endolysin is a phage phi11, phage Twort, phage P68, phage phiWMY or phage K endolysin (eg, MV-L endolysin or P-27/HP endolysin).

The de-repressor may act as an activator that is capable of activating the CRISPR/Cas system in the cell, wherein the activator activates said system in the presence of the repressor. For example, the repressor may be bound to a nucleic acid, such as a promoter, of the system and the activator over-rides the repression and activates the system when the repressor is bound to an element of the system.

In an example, the protospacer sequence is comprised by a chromosome of the host cell, eg, wherein the sequence is comprised by an antibiotic resistance gene, virulence gene or essential gene of the host cell. An example, provides the vector(s) of the invention in combination with an antibiotic agent (eg, a beta-lactam antibiotic), eg, wherein the vector(s) target a protospacer sequence comprised by an antibiotic resistance gene comprised by host cell genome or episome (eg, a plasmid comprised by the host cell(s)). In an example, the episome is a plasmid, transposon, mobile genetic element or viral sequence (eg, phage or prophage sequence).

In an example, the target sequence is a chromosomal sequence, an endogenous host cell sequence, a wild-type host cell sequence, a non-viral chromosomal host cell sequence, not an exogenous sequence and/or a non-phage sequence (ie, one more or all of these), eg, the sequence is a wild-type host chromosomal cell sequence such as a antibiotic resistance gene or essential gene sequence comprised by a host cell chromosome. In an example, the sequence is a host cell plasmid sequence, eg, an antibiotic resistance gene sequence.

Optionally, the or each host cell protospacer sequence is a adjacent a NGG, NAG, NGA, NGC, NGGNG, NNGRRT or NNAGAAW protospacer adjacent motif (PAM), eg, a AAAGAAA or TAAGAAA PAM (these sequences are written 5′ to 3′). In an embodiment, the PAM is immediately adjacent the 3′ end of the protospacer sequence. In an example, the Cas is a S aureus, S thermophilus or S pyogenes Cas. In an example, the Cas is Cpf1 and/or the PAM is TTN or CTA.

Optionally, the system is a Type I (eg, Type I-A, I-B, I-C, I-D, I-E, or I-F) CRISPR/Cas system. Optionally, the system is a Type II CRISPR/Cas system. Optionally, the system is a Type IIII CRISPR/Cas system. Optionally, the system is a Type IV CRISPR/Cas system. Optionally, the system is a Type V CRISPR/Cas system. Optionally, the system is a Type VI CRISPR/Cas system.

Optionally, the CRISPR array comprises multiple copies of the same spacer for targeting the protospacer sequence. Optionally, there is provide a vector or plurality of vectors of the invention, wherein the vector(s) comprises a plurality of CRISPR arrays of said gRNA-encoding sequences for host cell protospacer sequence targeting. Optionally, the or each vector comprises two, three or more of copies of nucleic acid sequences encoding crRNAs (eg, gRNAs), wherein the copies comprise the same spacer sequence for targeting a host cell sequence (eg, a virulence, resistance or essential gene sequence).

In an example, at least two target sequences are modified by Cas, for example an antibiotic resistance gene and an essential gene. Multiple targeting in this way may be useful to reduce evolution of escape mutant host cells.

In an example, the Cas is a wild-type endogenous host cell Cas nuclease. In an example, protospacer target modification or cutting is carried out by a dsDNA Cas nuclease (eg, a Cas9, eg, a spCas9 or saCas9), whereby repair of the cut is by non-homologous end joining (NHEJ); alternatively the Cas is an exonuclease or Cas3. In an example, the Cas is a Cas nuclease for cutting, dead Cas (dCas) for interrupting or a dCas (eg, dCas3 or dCas9) conjugated to a transcription activator for activating the target.

In an example, the array, gRNA-encoding sequence or vector is not in combination with a Cas endonuclease-encoding sequence that is naturally found in a cell together with repeat sequences of the array or, gRNA-encoding sequence.

A tracrRNA sequence may be omitted from an array or vector of the invention, for example for Cas systems of a Type that does not use tracrRNA, or an endogenous tracrRNA may be used with the crRNA encoded by the vector.

In an example, the host protospacer sequence comprises at least 5, 6, 7, 8, 9, 10, 20, 30 or 40 contiguous nucleotides.

In an example, the or each vector comprises an exogenous promoter functional for transcription of the crRNA or gRNA in the host.

In an example, the or each array repeats are identical (or at least 90, 95 or 98% identical) to a repeat in a host array comprised by the CRISPR/Cas system of the host, wherein the vector array does not comprise a PAM recognised by a Cas nuclease of the host CRISPR/Cas system. This applies mutatis mutandis to repeat sequence of the gRNA. This is advantageous since it simply enables the CRISPR array to use the endogenous host Cas to target the host target sequence. This then is efficient as the array is tailored for use by the host machinery, and thus aids functioning in the host cell. Additionally, or alternatively this enables the vector-encoded array sequence to combine with endogenously-encoded tracrRNA, since the CRISPR array repeats will hybridise to the endogenous tracrRNA for the production of pre-crRNA and processing into mature crRNA that hybridises with the host target sequence. The latter complex can then guide the endogenous Cas nuclease (eg, Cas3). This embodiment therefore provides the flexibility of simply constructing a vector (eg, packaged virus or phage) containing the CRISPR array but not comprising a tracrRNA- and/or Cas nuclease-encoding sequence. This is more straightforward for vector construction and also it frees up valuable space in the vector (eg, virus or phage) which is useful bearing in mind the capacity limitation for vectors, particularly viral vectors (eg, phage). The additional space can be useful, for example, to enable inclusion of many more spacers in the array, eg, to target the host genome for modification, such as to inactivate host genes or bring in desired non-host sequences for expression in the host. Additionally or alternatively, the space can be used to include a plurality of CRISPR arrays in the vector. These could, for example, be an arrangement where a first array is of a first CRISPR/Cas type (eg, Type II or Type II-A) and the second array could be of a second type (eg, Type I or III or Type II-B). Additionally or alternatively, the arrays could use different Cas nucleases in the host (eg, one array is operable with the host Cas nuclease and the second array is operable with an exogenous Cas nuclease (ie, a vector-encoded nuclease) or a different host Cas). These aspects provide machinery for targeting in the host once the vector has been introduced, which is beneficial for reducing host resistance to the vector, as the host would then need to target a greater range of elements. For example, if the host were able to acquire a new spacer based on the first CRISPR array sequence, the second CRISPR array could still function in the host to target a respective target sequence in the host cell. Thus, this embodiment is useful to reduce host adaptation to the vector.

Optionally, the vector of the invention comprises one, two, three, four, five, six or more CRISPR arrays or gRNA-encoding sequences of the invention comprising a plurality (eg, 2, 3, 4 5, 10, 15, 20, 25, 30, 35, 40, 45, 50, 55, 60, 70, 80, 90, 100 or more) copies of a spacer for hybridising to a host target sequence. This reduces the chances of all of these spacers being lost by recombination in the host cell. In a further application of this aspect, the CRISPR arrays comprise a first array comprising one or more (eg, 2, 3, 4 5, 10, 15, 20, 25, 30, 35, 40, 45, 50, 55, 60, 70, 80, 90 or more) of the spacer copies and a second array comprising one or more (eg, 2, 3, 4 5, 10, 15, 20, 25, 30, 35, 40, 45, 50, 55, 60, 70, 80, 90 or more) of the identical spacer copies, wherein spacer copies in the first array are each flanked by first repeats and the identical spacer copies in the second array are each flanked by second repeats, wherein the first repeats are different from the second repeats. This has the benefit the first repeats can be selected to be recognised by a first (eg, de-repressed) host Cas nuclease, and the second repeats are recognised by a second Cas (eg, a vector-encoded or host Cas) to reduce the chances of host adaptation involving more than one of the arrays.

Optionally, there is provide a vector or plurality of vectors of the invention, wherein each vector is a plasmid, cosmid, virus, a virion, phage, phagemid or prophage. For example, the invention provides a plurality of bacteriophage comprising a plurality of vectors of the invention, eg, wherein the vectors are identical. In an example, the vector is a viral vector. Viral vectors have a particularly limited capacity for exogenous DNA insertion, thus virus packaging capacity needs to be considered. Room needs to be left for sequences encoding vital viral functions, such as for expressing coat proteins and polymerase. In an example, the vector is a phage vector or an AAV or lentiviral vector. Phage vectors are useful where the host is a bacterial cell. In an example, the vector is a virus capable of infecting an archaea host cell.

Optionally, vector components (a) and (b) are comprised by a transposon that is capable of transfer into and/or between host cells. The transposon can be a transposon as described in US20160333348, GB1609811.3 and all US equivalent applications; the disclosures of these, including these specific transposon disclosures, are incorporated herein in its entirety and for potential inclusion of one or more disclosures therein in one or more claims herein.

In an example, the or each vector is provided by a nanoparticle or in liposomes.

In an example, transcription of one or more components of the CRISPR/Cas system is repressed. For example, transcription of one or more Cas sequences (eg, Cas3, Cas9 or Cpf1) is repressed. For example, transcription of one or more of CasA, B, C, D and E of a Type I CRISPR/Cas system is repressed, eg, CasA and/or Cas3 is repressed.

Optionally, Cas modification of the host cell genome
a. kills the host cell;
b. reduces growth or proliferation of the cell or episome;
c. increases growth or proliferation of the cell or episome;
d. reduces or prevents transcription of a nucleotide sequence that comprises or is adjacent a said protospacer sequence; or
e. increases transcription of a nucleotide sequence that comprises or is adjacent a said protospacer sequence.

In an example, inhibition of host cell population growth is at least 2, 3, 4, 5, 6, 7, 8, 9 or 10-fold compared to the growth of said host cells not exposed to vectors of the invention. For example, growth inhibition is indicated by a lower bacterial colony number of a first sample of host cells (alone or in a mixed bacterial population) by at least 2, 3, 4, 5, 6, 7, 8, 9 or 10-fold compared to the colony number of a second sample of the host cells (alone or in a mixed bacterial population), wherein the first sample of cells have been transformed by said vectors but the second sample has not been exposed to said vectors. In an embodiment, the colony count is determined 12, 24, 36 or 48 hours after the first sample has been exposed to the vectors of the invention. In an embodiment, the colonies are grown on solid agar in vitro (eg, in a petri dish). It will be understood, therefore, that growth inhibition can be indicated by a reduction (<100% growth compared to no treatment, ie, control sample growth) in growth of cells or populations comprising the target sequence, or can be a complete elimination of such growth. In an example, growth of the host cell population is reduced by at least 10, 20, 30, 40, 50, 60, 70, 80, 90 or 95%, ie, over a predetermined time period (eg, 24 hours or 48 hours following combination with the crRNA or gRNA in the host cells), ie, growth of the host cell population is at least such percent lower than growth of a control host cell population that has not been exposed to said vectors, but otherwise has been kept in the same conditions for the duration of said predetermined period. In an example, percent reduction of growth is determined by comparing colony number in a sample of each population at the end of said period (eg, at a time of mid-exponential growth phase of the control sample). For example, after exposing the test population to the vectors at time zero, a sample of the test and control populations is taken and each sample is plated on an agar plate and incubated under identical conditions for said predetermined period. At the end of the period, the colony number of each sample is counted and the percentage difference (ie, test colony number divided by control colony number and then times by 100, and then the result is subtracted from 100 to give percentage growth reduction). The fold difference is calculated by dividing the control colony number by the test colony number.

Inhibition of population growth can be indicated, therefore, by a reduction in proliferation of host cell number in the population. This may be due to cell killing by the de-repressed or activated CRISPR/Cas system and/or by downregulation of host cell proliferation (eg, division and/or cell growth) by the action of the system on the target protospacer sequence in host cells. In an embodiment of a method, use, treatment or prevention as disclosed herein, host cell burden of the human or animal subject or environment is reduced, whereby the disease or condition is treated (eg, reduced or eliminated) or prevented (ie, the risk of the subject developing the disease or condition) is reduced or eliminated or the environment is treated.

In an example, components (a) and (b) are instead comprised by first and second vectors that are different, for introduction of the vectors into the host cell wherein said Cas modification takes place.

In an example, the de-repressor is a protein or an RNA. For example, the de-repressor is a silencing RNA (siRNA) that is complementary to a nucleotide sequence comprised by a host cell gene encoding the repressor, eg, wherein the sequence is an ORF or a sequence of a regulatory element, such as a promoter or enhancer, of the gene.

In an example, the repressor is an anti-CRISPR or anti-Cas (eg, anti-Cas3 or anti-Cas9) protein, nucleic acid or RNA, eg, encoded by a prophage comprised by the host cell. For example, the repressor is encoded by a acr, acrIIA2 and acrIIA4, aca1 and aca2 gene or orthologue, homologue or paralogue thereof; and optionally the de-repressor is a siRNA that is complementary to the de-repressor gene sequence in the host cell, thereby silencing the expression of the gene. In an example, the repressor is an AcrIIA protein, eg, AcrIIA2 and/or AcrIIA4.

Because H-NS often acts in combination with other nucleoid-associated proteins (NAPs), the binding of related regulatory proteins, such as StpA, LRP and FIS (Luijsterburg et al., 2006; Dorman, 2009) has been analysed. The repressor may be H-NS (nucleoid-structuring protein), StpA, FIS, LRP ((leucine-responsive regulatory protein) or CRP (cAMP receptor protein); or an orthologue, or paralogue, homologue or functional equivalent thereof that acts as a repressor in the host cell. For example, the repressor may be an orthologue, or paralogue, homologue or functional equivalent of an E coli H-NS, StpA, FIS, LRP or CRP protein. In an example, the CRISPR/Cas system is repressed by more than one such repressor, eg, H-NS and LRP; or H-NS and CRP.

The de-repressor may be a mutant H-NS, StpA, LRP or CRP that is capable of forming a complex with H-NS, StpA, LRP or CRP repressor (eg, wild-type host or E coli H-NS, StpA, LRP or CRP) respectively in the host cell to prevent or reduce repression of the CRISPR/Cas system. The de-repressor may be a mutant H-NS, StpA, LRP or CRP that is capable of forming a complex with wild-type host or E coli H-NS, StpA, LRP or CRP respectively (eg, in vitro or in a host cell or in E coli).

In an example, the repressor is H-NS or StpA and the de-repressor is LeuO.

In an example, the episome is a plasmid.

The following definitions apply:—

### Homologue

A gene or protein related to a second gene or protein by descent from a common ancestral DNA sequence. The term, homologue, may apply to the relationship between genes or their protein products separated by the event of speciation (see orthologue) or to the relationship between genes separated by the event of genetic duplication (see paralogue).

Orthologue
Orthologues are genes or proteins in different species that evolved from a common ancestral gene or protein by speciation. Normally, orthologues retain the same function in the course of evolution.

Paralogue
Paralogues are genes or proteins related by duplication within a genome. Orthologues retain the same function in the course of evolution, whereas paralogues evolve new functions, even if these are related to the original one.

A homologue of a repressor or de-repressor itself has activity as a repressor or de-repressor in a host cell. An orthologue of a repressor or de-repressor itself has activity as a repressor or de-repressor in a host cell. An paralogue of a repressor or de-repressor itself has activity as a repressor or de-repressor in a host cell.

In an example, the cell is a bacterial or archaeal cell. In an example, the cell is comprised by an environment, soil, plant, mammal, human, mouse, rat, pig, dog, primate, monkey, sheep, cow, horse, cat, ruminant, livestock, insect or a bird, eg, a chicken or turkey, such as comprised by a microbiome thereof. The microbiome may be a plant leaf, plant stem, soil, gut, skin, oral, lung, ocular, ear, tongue, armpit, vagina, rectal, scrotal, penile or hair microbiome. In an example, the cell is a vertebrate, invertebrate, mammal, human, rodent, mouse, rat, fish or insect cell.

In an example, the host cell(s) are E coli cell(s), eg, selected from
Shiga toxin-producing E. coli (STEC) (STEC may also be referred to as Verocytotoxin-producing E. coli (VTEC) or enterohemorrhagic E. coli (EHEC). This pathotype is the one most commonly heard about in the news in association with foodborne outbreaks);
Enterotoxigenic E. coli (ETEC);
Enteropathogenic E. coli (EPEC);
Enteroaggregative E. coli (EAEC);
Enteroinvasive E. coli (EIEC); and
Diffusely adherent E. coli (DAEC).

The strain of Shiga toxin-producing E. coli O104:H4 that caused a large outbreak in Europe in 2011 was frequently referred to as EHEC. The most commonly identified STEC in North America is E. coli O157:H7. In an example, the cell(s) are E. coli O104:H4 or E. coli O157:H7.

It has been observed that endogenous CRISPR/Cas systems may be somewhat de-repressed and/or upregulated in host cells in stationary phase, eg, to combat phage invasion or plasmid horizontal transfer when cells are densely packed. In an example of the vector of the invention component (a) and/or (b) is operably linked to a promoter for expression in host cells in stationary growth phase. Densely packed cells may be present in biofilms, and thus, in an example of the vector of the invention component (a) and/or (b) is operably linked to a promoter for expression in host cells comprised by a biofilm (eg, in an environment or in a human or animal body, such as a lung or gut biofilm).

To enable protospacer targeting also or alternatively in the exponential growth phase (eg, where endogenous CRISPR/Cas systems may be repressed),in an example of the vector of the invention component (a) and/or (b) is operably linked to a promoter for expression in host cells in exponential growth phase. In an example of the vector of the invention component (a) and/or (b) is operably linked to a promoter for expression in host cells in lag phase.

The transcriptional regulator CsgD is central to biofilm formation, controlling the expression of the curli structural and export proteins, and the diguanylate cyclase, adrA, which indirectly activates cellulose production. Chirwa NT and Herrington MB, Microbiology. 2003 February; 149(Pt 2):525-35, “CsgD, a regulator of curli and cellulose synthesis, also regulates serine hydroxymethyltransferase synthesis in Escherichia coli K-12” explains that the homologous CsgD and AgfD proteins are members of the FixJ/UhpA/LuxR family and are proposed to regulate curli (thin aggregative fibres) and cellulose production by Escherichia coli and Salmonella enterica serovar Typhimurium, respectively. It is proposed that CsgD upregulates glyA to facilitate synthesis of curli. In an example of the vector of the invention component (a) and/or (b) is operably linked to a promoter for expression in the host cell, wherein the promoter is controlled by CsgD or AgfD, eg, an E coli CsgD or AgfD. This may be useful, for example, to promote expression of the vector component in host cells in biofilms or involved in biogenesis of biofilms. Optionally the host cell is an Escherichia coli and Salmonella enterica serovar Typhimurium cell.

The gene rpoS (RNA polymerase, sigma S) encodes the sigma factor sigma-38 (σ38, or RpoS), a 37.8 kD protein in Escherichia coli. Sigma factors are proteins that regulate transcription in bacteria. Sigma factors can be activated in response to different environmental conditions. rpoS is transcribed in late exponential phase, and RpoS is the primary regulator of stationary phase genes. RpoS is a central regulator of the general stress response and operates in both a retroactive and a proactive manner: it not only allows the cell to survive environmental challenges, but it also prepares the cell for subsequent stresses (cross-protection). In an example of the vector of the invention component (a) and/or (b) is operably linked to a promoter for expression in the host cell, wherein the promoter is regulated by a sigma factor, such as RpoS, eg, an E coli RpoS. This may be useful, for example, to promote expression of the vector component in host cells in growth phases where sigma factor and RpoS regulation is upregulated. Optionally the host cell is an Escherichia coli and Salmonella enterica serovar Typhimurium cell. Transcription of rpoS in E. coli is mainly regulated by the chromosomal rpoSp promoter. rpoSp promotes transcription of rpoS mRNA, and is induced upon entry into stationary phase in cells growing on rich media. Thus, in one example the or each said promoter comprised by the vector operates to upregulate transcription of its component (a) or (b) in host cells in stationary phase, eg, the or each promoter is a rpoSp promoter.

As a defence mechanism, the bacterial host environments are hostile to invading pathogens, such as phage. Therefore, infection can be a stressful event for pathogenic bacteria and control of virulence genes may be temporally correlated with the timing of infection by pathogens. Discovery of RpoS-dependent virulence genes in Salmonella are consistent with RpoS as a general regulator of the stress response: the spy gene found on a virulence plasmid in this bacterium is controlled by RpoS, and interestingly, required for growth in deep lymphoid tissue such as the spleen and liver. Thus, in an embodiment, the vector is a virus, eg, a bacteriophage that is capable of infecting the host cell (eg, an Escherichia coli or Salmonella cell and component (a) and/or (b) is operably linked to a promoter for expression in the host cell, wherein the promoter is regulated by RpoS, eg, an E. coli RpoS. Optionally, the host cell(s) is comprised by a spleen or liver bacterial population comprised by a human or animal. Optionally, the vector is for administration to said human or animal to treat or prevent a condition or disease, eg, a spleen, liver or immune-related disease or condition. In an example, the or each promoter is a promoter of a virulence gene, eg, a host cell virulence gene, eg, a spy gene.

Optionally, nucleotide sequence (a) comprises a constitutive promoter or strong promoter for expression of the sequence in the host cell.

Optionally, nucleotide sequence or array (b) comprises a constitutive promoter or strong promoter for expression of the sequence or array in the host cell.

Optionally, (a) and (b) are comprised by the same operon or under the control of a common expression control (eg, same promoter) that is operable in the cell.

Optionally, the promoter is a strong and/or constitutive promoter for expression in the host cell.

Optionally, the host cell is a wild-type host cell, for example, wherein the vector is for use in a natural environment or human or animal microbiome.

Optionally, the vector comprises an expressible htpG sequence, eg, wherein the host is an E. coli host, such as comprised by a human or animal microbiome. HtpG increases steady-state Cas3 protein levels in E. coli at 37 degrees C. This embodiment is therefore particularly useful for modifying host cells, such as E coli, comprised by humans or animals (eg, comprised by a gut microbiome thereof). In an example, the repressed Cas is a Cas3.

In an embodiment, there is provided a nucleic acid vector for introduction into a host cell, wherein the host cell comprises a CRISPR/Cas system comprising Cascade and Cas3, wherein the Cascade is repressed (eg, by H-NS) in the host cell and the vector comprises
(i) An expressible nucleotide sequence encoding a de-repressor (eg, LeuO) of said Cascade repression; and
(ii) An expressible nucleotide sequence encoding a Cas3, wherein the Cas 3 is capable of functioning with de-repressed Cascade in the host cell;
   Wherein the nucleotide sequences are capable of being expressed in the host cell, whereby the de-repressor de-represses or activates the Cascade, whereby the Cascade functions with the Cas3 to modify a protospacer sequence of the host cell genome or to modify a protospacer sequence of an episome comprised by the host in the presence of the de-repressor;
Or
A nucleic acid vector for introduction into a host cell, wherein the host cell comprises a CRISPR/Cas system comprising Cascade and Cas3, wherein the Cascade is repressed (eg, by H-NS) in the host cell, the vector comprising
(i) An expressible nucleotide sequence encoding a de-repressor (eg, LeuO) of said Cascade repression; and
(ii) An expressible nucleotide sequence encoding a Cas3, wherein the Cas 3 is capable of functioning with de-repressed Cascade in the host cell;
   Wherein the nucleotide sequences are capable of being expressed in the host cell, whereby the de-repressor de-represses or activates the Cascade, whereby the Cascade functions with the Cas3 to modify a protospacer sequence of the host cell genome or to modify a protospacer sequence of an episome comprised by the host in the presence of the de-repressor.

Optionally, the nucleotide sequences of (i) and (ii) are comprised by the same operon or under the control of a common expression control (eg, same promoter) that is operable in the cell.

Optionally, sequences (i) and (ii) and the CRISPR array or sequence encoding said gRNA are comprised by two or more different vectors for introduction into the host cell for expression of the de-repressor, Cas3 and array or gRNA together in the host cell.

An aspect provides:—

A nucleic acid vector (optionally according to any other configuration, example, embodiment or aspect of the invention) for introduction into a bacterial or archaeal host cell, wherein the cell comprises an endogenous CRISPR/Cas system that is naturally repressed by a repressor in the cell, the vector comprising
(a) A nucleotide sequence encoding a de-repressor that is capable of de-repressing the CRISPR/Cas system in the cell, wherein the sequence is expressible in the cell to produce the de-repressor; and
(b) A CRISPR array for production of one or more crRNAs in the cell; and/or one or more nucleotide sequences each encoding a respective guide RNA (gRNA, eg, a single guide RNA) in the cell; wherein each crRNA or gRNA is capable of guiding Cas to modify a protospacer sequence of the host cell genome or to modify a protospacer sequence of an episome comprised by the host in the presence of the de-repressor; Wherein the repressor is H-NS, StpA, LRP or CRP or a homologue, or an orthologue or a functional equivalent thereof (eg, a homologue, orthologue or functional equivalent of an E coli K12 H-NS, StpA, LRP or CRP) encoded by the cell genome; and Wherein the de-repressor is LeuO or a homologue, or an orthologue or a functional equivalent thereof (eg, a homologue, orthologue or functional equivalent of an E coli K12 LeuO) that is capable of forming a complex with the repressor or an E coli K12 H-NS, StpA, LRP or CRP; or a mutant of H-NS, StpA, LRP or CRP (eg, a mutant of an E coli K12 H-NS, StpA, LRP or CRP) that is capable of forming a complex with the repressor.

In an example, the repressor inhibits, sterically blocks or binds one or more sequences of the CRISPR/Cas system (eg, a promoter sequence, eg, the promoter of the repressed Cas) to reduce or prevent transcription of Cas in the cell. For example, the promoter sequence is a Cas (eg, CasA or Cas3) gene promoter. In an example, the repressor is capable of competing with said H-NS, StpA or CRP for binding to a Type I CasA (cse1) promoter. In an example, the repressor is capable of competing with said H-NS, StpA or CRP for binding to a Type I (eg, Type I-B or -F) Cas3 promoter For example, this can be determined in vitro using a standard competition assay, such as surface plasmon resonance (SPR) or ELISA. In an example, the repressor Inhibits, sterically blocks or binds a σ⁷⁰-dependent promoter sequence, a P_{cispr1} promoter sequence, a P_{cas} promoter sequence and/or an anti-P_{cas} promoter sequence, eg, wherein the host cell is an E. coli cell.

In an example, the de-repressor is a LysR-type regulator protein. In an example, the de-repressor is capable of competing with LeuO for binding to a Type I CasA (cse1) or Cas3 promoter (eg, such a promoter from E. coli K12). For example, this can be determined in vitro using a standard competition assay, such as SPR or ELISA. In an example, the repressor is H-NS and the de-repressor is H-NS^{G113D}.

In an example, the repressor binds a CasA promoter comprised by a CRISPR array of a CRISPR/Cas system of the host cell species, wherein the cas is said repressed Cas, eg, CasA or Cas3.

A σ⁷⁰-dependent promoter has been observed in E. coli about 50 bp upstream of the first (5′-most) nucleotide from the first CRISPR repeat sequence of a Type I array. The DNase footprint demonstrates the existence of a σ⁷⁰-dependent promoter, located between positions-40 to -90, which is termed Pcrispr1. Thus, in an example, the repressor binds to a sequence of a Binding of σ⁷⁰-dependent promoter comprised by a CRISPR array of a CRISPR/Cas system of the host cell. In an example, the repressor inhibits σ⁷⁰ RNA polymerase transcription of a CRISPR array of a CRISPR/Cas system of the host cell.

In an example, the de-repressor is a H-NS paralogue, eg, as further discussed below.

StpA, which is a paralogue of H-NS with 58% amino acid identity (Zhang and Belfort, 1992), shows a very similar DNA-binding characteristic as H-NS, producing the same large region of DNase I protection in its target binding site. Consistent with a generally higher affinity for DNA (Zhang et al., 1996) StpA reaches complete protection at somewhat lower concentrations than H-NS. LRP and FIS cause weaker protection from DNase I cleavage. In an example, the repressor is a nucleoid-associated protein (NAP). In an example, the de-repressor is a mutant of a nucleoid-associated protein (NAP), wherein the mutant competes with the NAP for binding to a target binding site of the NAP (eg, a IGLB sequence), such as a binding site comprised by a CRISPR array of a CRISPR/Cas system of the host cell species, wherein the Cas is said repressed Cas, eg, Cas3 or CasA). In an example, the NAP is StpA, LRP, FIS or H-NS. In an example, the repressor is H-NS and the de-repressor is LRP and/or FIS expressed from the vector(s), eg, by under the control of a strong or constitutive promoter, for expression in the host cell(s). In this way, the de-repressor may be expressed in excess that out-competes H-NS for binding to the target binding site, and yet the de-repressor may not repress or only weakly repress the Cas or Cascade activity in the host cell. The promoter (or any other promoter herein) may, for example, be the bacterial constitutive promoter OXB17, OXB18, OXB19 or OXB20, preferably the latter as it is the strongest (see http://www.oxfordgenetics.com/Products/Plasmids/Details/Bacterial/pSF-OXB18/OG561). In an example, the promoter is a T7 promoter and the vector(s) encode T7 RNA polymerase for expression in the host cell.

Numerous factors influencing the H-NS silencing and antisilencing have been documented in the past (Navarre et al., 2007; Stoebel et al., 2008) including for instance SlyA (Lithgow et al., 2007; Perez et al., 2008). SlyA, as some other related proteins, do not act themselves as regulators interacting with DNA target sites but rather form DNA-binding defective heterodimers with H-NS, thereby counteracting H-NS-mediated silencing. In an example, therefore, the de-repressor comprises SlyA. In an example, the de-repressor comprises PhoP, PhoQ, Crp and/or Fnr. A variety of anti-silencing mechanisms have been observed involving (i) protein-independent processes that operate at the level of local DNA structure, (ii) DNA-binding proteins such as Ler, LeuO, RovA, SlyA, VirB, and proteins related to AraC, and (iii) modulatory mechanisms in which H-NS forms heteromeric protein-protein complexes with full-length or partial paralogues such as StpA, Sfh, Hha, YdgT, YmoA or H-NST. The RovA protein is a homologue of SlyA that was identified originally as a positive regulator of inv, the gene coding for invasin, in response to temperature and growth phase in Yersinia (Cathelyn et al., 2007). RovA is now known to control the transcription of a regulon of genes that, like inv, are subject to repression by the H-NS protein. It has been proposed that the principal function of RovA in Yersinia enterocolitica is to act as an antagonist of H-NS-mediated transcriptional silencing (Cathelyn et al., 2007). In an example, therefore, the de-repressor comprises one, two, three or more of Ler, LeuO, RovA, SlyA, VirB, AraC, StpA, Sfh, Hha, YdgT, YmoA and H-NST; or a homologue, orthologue, paralogue or functional equivalent thereof.

The major virulence factors of V cholerae, the aetiological agent of Asiatic cholera, are encoded by genes within A+T-rich horizontally transmissible genetic elements (Davis & Waldor, 2003; McLeod et al., 2005; Murphy & Boyd, 2008). These genes are regulated by several environmental signals to ensure that their products are expressed when the bacterium arrives at appropriate sites in the host and that they are repressed elsewhere (Lee et al., 1999; Schild et al., 2007). Among the major virulence factors expressed by V. cholerae are cholera toxin, CTX, and the toxin co-regulated pilus, Tcp (Skorupski & Taylor, 1997). H-NS silences the transcription of the genes encoding these major virulence factors by targeting their A+T-rich promoters (Nye et al., 2000). This silencing is opposed by the ToxT regulatory protein, an AraC-like DNA-binding protein that derepresses transcription of a number of virulence gene promoters in V. cholerae (Yu & DiRita, 2002). The mechanism is thought to involve not only the displacement of H-NS but also the activation of transcription by ToxT, possibly due to direct interaction between ToxT and RNA polymerase (Hulbert & Taylor, 2002; Yu & DiRita, 2002). In an example, therefore, the de-repressor comprises ToxT (eg, V. cholerae ToxT). In an example, therefore, the de-repressor comprises AraC or a homologue, orthologue, paralogue or functional equivalent thereof. Examples of the latter are AppY, CfaD, GadW, GadX, HilC, HilD, PerA, RegA, Rns, UreR and VirF.

The ability to form nucleoprotein filaments with DNA plays an important role in H-NS-mediated transcriptional silencing. LeuO has been identified as a protein that can set limits to the polymerization of H-NS along the genetic material. It is a LysR-like DNA-binding protein that was identified as a transcription activator in the promoter relay that governs the expression of the leuABCD operon in Salmonella Typhimurium (Chen & Wu, 2005; Chen et al., 2005; Fang & Wu, 1998). In an example, therefore, the de-repressor comprises LysR or a homologue, orthologue, paralogue or functional equivalent thereof.

Other nucleoid-associated proteins can antagonize H-NS binding to DNA. Experiments with magnetic tweezers and atomic force microscopy have suggested that the abundant HU protein can compete with H-NS for the same binding sites in DNA, opening up H-NS-condensed promoter regions (van Noort et al., 2004). The Fis protein has also been reported to antagonize H-NS repression, for example at rRNA gene promoters where its binding sites are distributed among those of H-NS (Schneider et al., 2003). At later stages of growth when Fis levels are low, H-NS represses the rRNA gene promoters (Afflerbach et al., 1998). The nucleoid-associated protein HU and the RpoS stress and stationary-phase sigma factor of RNA polymerase have been described as having positive regulatory roles at the H-NS-repressed proU promoter in E. coli (Manna & Gowrishankar, 1994), and a wider overlap between the H-NS and RpoS regulons has been described (Barth et al., 1995). This may indicate a role for RpoS in overcoming H-NS-mediated repression in bacteria undergoing stress. In an example, therefore, the de-repressor comprises HU, RpoS and/or Fis; or a homologue, orthologue, paralogue or functional equivalent thereof.

An intriguing group of proteins is made up of small polypeptides with homology to the oligomerisation domain of H-NS. Those with the closest amino acid sequence similarity to this domain are members of the H-NST family, so-called because they resemble H-NS truncates that lack the nucleic acid binding and linker domains (Williamson & Free, 2005). The genes coding for these truncates have been detected in pathogenicity islands of various pathogenic enterobacteria including enteropathogenic E. coli (EPEC) and uropathogenic E. coli. The protein from EPEC, H-NST(EPEC), co-purifies with H-NS. This protein can interfere with the ability of H-NS to repress the proU operon in E. coli. In an example, therefore, the de-repressor comprises H-NST; or a homologue, orthologue, paralogue or functional equivalent thereof; eg, wherein the repressor is H-NS or StpA.

Genes coding for small proteins that interact directly with H-NS are found in the ancestral chromosome and on horizontally acquired islands. The YmoA protein of Yersinia was recognized originally as a regulator of virulence gene expression in Y enterocolitica (Cornelis et al., 1991). It is related to the Hha protein, discovered initially as a modulator of haemolysin gene expression in E coli, and the two proteins can substitute for one another functionally (Balsalobre et al., 1996; Mikulskis & Cornelis, 1994). The Hha protein must interact with H-NS in order to exert its effect on haemolysin gene expression; YmoA also interacts with H-NS and this relationship was exploited in the isolation of the H-NS protein from Yersinia (Nieto et al., 2000, 2002). The solution structure of YmoA has been solved using nuclear magnetic resonance spectroscopy (McFeeters et al., 2007). The results lend weight to the view that YmoA (and Hha) should be regarded as independent oligomerisation domains of H-NS. Potentially, the proteins may oligomerise to produce YmoA-H-NS and Hha-H-NS heteromers. The absence of a nucleic acid-binding domain on the YmoA and Hha partners may result in a failure of the heteromers to participate in DNA-protein-DNA bridging, compromising (or at least modifying) the structure of repression complexes. The discovery of paralogues of Hha-like proteins has added a further layer of complexity. The ydgT gene codes for an Hha-like protein in E. coli and Salmonella, and it can interact with H-NS and the H-NS paralogue, the StpA protein (Paytubi et al., 2004). In an example, therefore, the de-repressor comprises YmoA and/or Hha and/or ydgT; or a homologue, orthologue, paralogue or functional equivalent thereof; eg, wherein the repressor is H-NS or StpA.

Not all H-NS paralogues are thought to act by direct protein-protein interaction with H-NS. The Ler DNA-binding protein is encoded by the LEE (locus of enterocyte effacement) pathogenicity island of enterohaemorrhagic E. coli (EHEC) and EPEC. It activates the transcription of the major virulence operons in the island at 37° C. by opposing the silencing activity of H-NS (Barba et al., 2005; Bustamante et al., 2001; Haack et al., 2003; Umanski et al., 2002). Ler and H-NS are partial paralogues whose oligomerisation domains are highly divergent coiled-coils; there is no evidence that Ler and H-NS form heterodimers. Instead, Ler is thought to displace H-NS (Haack et al., 2003). It also acts on gene expression outside the LEE (Elliott et al., 2000). For example, Ler counteracts the silencing activity of H-NS at the 1 pf operon in EHEC, which encodes long polar fimbriae (Torres et al., 2007). Thus, despite its homology to H-NS, Ler acts more like VirB or SlyA. In an example, therefore, the de-repressor comprises Ler; or a homologue, orthologue, paralogue or functional equivalent thereof; eg, wherein the repressor is H-NS or StpA.

The gp5.5 protein from bacteriophage T7 is known to bind and inactivate H-NS, thereby supporting the propagation of the phage (Liu and Richardson, 1993). In an example, therefore, the de-repressor comprises The gp5.5; or a homologue, orthologue, paralogue or functional equivalent thereof; eg, wherein the repressor is H-NS or StpA.

One might ask, why the CRISPR-cas system is cryptic. Because the Cas proteins are involved in the integration of foreign DNA spacers the cell must avoid that host DNA elements are erroneously integrated. The constant expression of cas genes might certainly be detrimental to the cell. Therefore mechanisms must exist, which keep cas gene expression silent until needed. Our data suggest that H-NS is at least one important component responsible for this kind of control. Thus, in one configuration, the invention provides any vector (or use or method using such vector, or composition) disclosed herein except wherein the vector(s) does not comprise the nucleotide sequence that encodes a crRNA or gRNA or does not comprise a CRISPR array, but wherein the vector(s) encodes one or more of said de-repressors (eg and the repressor is H-NS). In this configuration, expression of the de-repressor in the cell de-represses or activates endogenous Cas expression in the host cell (eg, Cas3 and/or Cascade Cas, such as CasA) and such expression causes modification (eg, cutting of host DNA, such as chromosomal DNA) which kills the cell(s) or reduces cell growth or proliferation. It may be advantageous for each de-repressor to be encoded in the vector(s) from a strong and/or constitutive promoter for expression in the host cell. High levels of de-repressor expression may displace the repressor, such as H-NS, and cause endogenous Cas to cut the chromosome or other DNA of the host cell, thereby killing the host cell or reducing its growth or proliferation.

For example, the vector does not comprise a CRISPR array for production of one or more crRNAs in the cell; and does not comprise one or more nucleotide sequences each encoding a respective guide RNA (gRNA, eg, a single guide RNA) in the cell. For example, the vector does not encode a crRNA or gRNA. This may be useful where de-repression is sufficient to activate endogenous Cas nuclease activity in the host cell, whereby the nuclease activity kills the host cell or inhibits host cell growth or proliferation. For example, the repressor is H-NS or StpA and the de-repressor is LeuO or any other de-repressor disclosed herein. For example, the expression of the de-repressor is under the control of a strong and/or constitutive promoter for expression in the host cell.

In an example the invention provides:—

A method of treating or preventing a disease or condition in a human or animal subject, the method comprising administering a vector to the subject, wherein host cells (eg, E. coli, Salmonella, or S. enteric serovar typhimurium) comprised by a microbiome of the subject are modified by endogenous de-repressed Cas of the cells, and the treatment or prevention is carried out; wherein
(i) each cell comprises a CRISPR/Cas system that is repressed by a repressor (eg, H-NS and/or StpA) in the cell (eg, a Cascade Cas, Cas3 or Cas9 is repressed),
(ii) the vector comprising an nucleotide sequence encoding a de-repressor (eg, LeuO) that is capable of de-repressing the CRISPR/Cas system in the cell, wherein the sequence is expressible in the cell to produce the de-repressor (eg, under the control of a strong and/or constitutive promoter); and
(iii) the vector being devoid of a CRISPR array for production of one or more crRNAs in the cell; and devoid of one or more nucleotide sequences encoding a respective guide RNA (gRNA, eg, a single guide RNA) in the cell.

A method of treating or preventing a disease or condition in a human or animal subject, the method comprising administering a vector to the subject, wherein host cells (eg, E. coli, Salmonella, or S. enteric serovar typhimurium) comprised by a microbiome of the subject are modified by endogenous de-repressed Cas of the cells, and the treatment or prevention is carried out; wherein
(i) each cell comprises a CRISPR/Cas system that is repressed by a repressor selected from H-NS and/or StpA in the cell, wherein a Cascade Cas, Cas3 or Cas9 is repressed,
(ii) the vector comprising an nucleotide sequence encoding a de-repressor that is capable of de-repressing the CRISPR/Cas system in the cell, wherein the sequence is expressible in the cell to produce the de-repressor under the control of a strong and/or constitutive promoter; and
(iii) the vector being devoid of a CRISPR array for production of one or more crRNAs in the cell; and devoid of one or more nucleotide sequences encoding a respective guide RNA (gRNA, eg, a single guide RNA) in the cell.

A method of killing a wild-type bacterial or archaeal cell (eg, E. coli or Salmonella cell), wherein the cell comprises an endogenous CRISPR/Cas system comprising nucleotide sequences encoding Cas3 and Cascade proteins, wherein Cas3 and/or Cascade is naturally repressed in the cell, the method comprising de-repressing said Cas3 and/or Cascade without introducing into the cell (i) a CRISPR array for production of one or more crRNAs in the cell; or (ii) one or more nucleotide sequences encoding a respective guide RNA (gRNA, eg, a single guide RNA); or without engineering the host cell to encode a crRNA or guide RNA. In an example, the method comprises introducing into the host cell a nucleic acid vector, wherein
(i) the cell comprises a CRISPR/Cas system that is repressed by a repressor selected from H-NS and/or StpA in the cell, wherein a Cascade Cas, Cas3 or Cas9 is repressed,
(ii) the vector comprising an nucleotide sequence encoding a de-repressor that is capable of de-repressing the CRISPR/Cas system in the cell, wherein the sequence is expressible in the cell to produce the de-repressor under the control of a strong and/or constitutive promoter; and
(iii) the vector being devoid of a CRISPR array for production of one or more crRNAs in the cell; and devoid of one or more nucleotide sequences encoding a respective guide RNA (gRNA, eg, a single guide RNA) in the cell.

A medicament comprising a plurality of nucleic acid vectors for introduction into host cells described herein, optionally further comprising one or more medical drugs (eg, an anti-cancer medicament) or antibiotics (eg, wherein the protospacer sequence is comprised by a host cell antibiotic resistance gene), for treating or preventing a disease or condition in a human or animal; wherein
(i) each cell comprises a CRISPR/Cas system that is repressed by a repressor selected from H-NS and/or StpA in the cell, wherein a Cascade Cas, Cas3 or Cas9 is repressed,
(ii) the vector comprising an nucleotide sequence encoding a de-repressor that is capable of de-repressing the CRISPR/Cas system in the cell, wherein the sequence is expressible in the cell to produce the de-repressor under the control of a strong and/or constitutive promoter; and
(iii) the vector being devoid of a CRISPR array for production of one or more crRNAs in the cell; and devoid of one or more nucleotide sequences encoding a respective guide RNA (gRNA, eg, a single guide RNA) in the cell.

In an example, the or each host cell (or first and/or second bacteria) is a gram positive cell. In an example, the or each host cell is an Enterobacteriaceae, eg, Salmonella, Yersinia pestis, Klebsiella, Shigella, Proteus, Enterobacter, Serratia, or Citrobacter cells. Optionally, the or each cell is an E coli (eg, E coli K12) or Salmonella (eg, S enteric serovar typhimurium) cell. Optionally, the or each host cell (or first and/or second bacteria) is a gram negative cell.

Optionally, the host (or first and/or second bacteria) is a mycoplasma, chlamydiae, spirochete or mycobacterium. Optionally, the host (or first and/or second bacteria) is a Streptococcus (eg, pyogenes or thermophilus) host. Optionally, the host (or first and/or second bacteria) is a Staphylococcus (eg, aureus, eg, MRSA) host. Optionally, the host (or first and/or second bacteria) is an E. coli (eg, 0157: H7) host. Optionally, the host (or first and/or second bacteria) is a Pseudomonas (eg, aeruginosa) host. Optionally, the host (or first and/or second bacteria) is a Vibrio (eg, cholerae (eg, 0139) or vulnificus) host. Optionally, the host (or first and/or second bacteria) is a Neisseria (eg, gonnorrhoeae or meningitidis) host. Optionally, the host (or first and/or second bacteria) is a Bordetella (eg, pertussis) host. Optionally, the host (or first and/or second bacteria) is a Haemophilus (eg, influenzae) host. Optionally, the host (or first and/or second bacteria) is a Shigella (eg, dysenteriae) host. Optionally, the host (or first and/or second bacteria) is a Brucella (eg, abortus) host. Optionally, the host (or first and/or second bacteria) is a Francisella host. Optionally, the host (or first and/or second bacteria) is a Xanthomonas host. Optionally, the host (or first and/or second bacteria) is a Agrobacterium host. Optionally, the host (or first and/or second bacteria) is a Erwinia host. Optionally, the host (or first and/or second bacteria) is a Legionella (eg, pneumophila) host. Optionally, the host (or first and/or second bacteria) is a Listeria (eg, monocytogenes) host. Optionally, the host (or first and/or second bacteria) is a Campylobacter (eg, jejuni) host. Optionally, the host (or first and/or second bacteria) is a Yersinia (eg, pestis) host. Optionally, the host (or first and/or second bacteria) is a Borelia (eg, burgdorferi) host. Optionally, the host (or first and/or second bacteria) is a Helicobacter (eg, pylori) host. Optionally, the host (or first and/or second bacteria) is a Clostridium (eg, dificile or botulinum) host. Optionally, the host (or first and/or second bacteria) is a Erlichia (eg, chaffeensis) host. Optionally, the host (or first and/or second bacteria) is a Salmonella (eg, typhi or enterica, eg, serotype typhimurium, eg, DT 104) host. Optionally, the host (or first and/or second bacteria) is a Chlamydia (eg, pneumoniae) host. Optionally, the host (or first and/or second bacteria) is a Parachlamydia host. Optionally, the host (or first and/or second bacteria) is a Corynebacterium (eg, amycolatum) host. Optionally, the host (or first and/or second bacteria) is a Klebsiella (eg, pneumoniae) host. Optionally, the host (or first and/or second bacteria) is a Enterococcus (eg, faecalis or faecim, eg, linezolid-resistant) host. Optionally, the host (or first and/or second bacteria) is a Acinetobacter (eg, baumannii, eg, multiple drug resistant) host.

Optionally, the de-repressed Cas is Cas3. Optionally, the de-repressed Cas is Cas9. Optionally, the de-repressed Cas is a Cascade Cas, eg, when the host cell is an E coli cell. Cascade is also known as CRISPR-associated complex for antiviral defence.

Optionally, the CRISPR/Cas system is a Type I (eg, Type I-B, Type I-E or Type I-F) system.

Optionally, the protospacer sequence is comprised by an essential gene, virulence gene or antibiotic resistance gene comprised by the cell.

Optionally, the vector comprises no sequences from the group consisting of CasA, B, C, D and E (eg, when the cell is an E coli cell) or CasABCDE12 (eg, when the host cell is a S. enterica serovar typhimurium cell) nucleotide sequences, or wherein the vector does not comprise all of the sequences of said group.

Optionally, the vector comprises no sequences from the group consisting of Cas1, Cas2, Cas5 and Cas6 sequences.

Optionally, the vector comprises no sequences from the group consisting of vector comprises no Cas 3 nucleotide sequence.

Optionally, the vector or any other aspect of the invention for medical use for treating or preventing a disease or condition in a human or animal subject, wherein the host cell is comprised by the subject. In an example, the host cell(s) is the disease (ie, an infection of the subject by the host cells) or is associated with or mediates a disease or condition (eg, IBD, colitis, Crohn's disease, a cancer, an autoimmune disease or condition, obesity, diabetes or a CNS disease or condition (eg, Alzheimer's disease or Parkinson's disease). Optionally, the vector or any other aspect of the invention for use in a medical method of treatment, prophylaxis, diagnosis of a human or animal body.

Optionally, the vector or any other aspect of the invention for reducing the growth or proliferation of host cell(s) in an environment (eg, soil, a composition comprising said host cells and yeast cells), human, animal or plant microbiome. This is useful, for example, when the microbiome is naturally-occurring.

Optionally, the vector or any other aspect of the invention for killing a plurality of host cells or for reducing the growth or proliferation thereof.

The or each host cell may be comprised by a microbiome (eg, gut microbiome or environmental microbiome) comprising a plurality of said host cells and comprising one or more cells of a species or strain (eg, bacterial species or strain, or archaeal species or strain) that is different from the species or strain of the host cells (eg, bacteria or archaea host cells).

An aspect provides a medicament comprising a plurality of vectors according to the invention, optionally further comprising one or more medical drugs (eg, an anti-cancer medicament) or antibiotics (eg, wherein the protospacer sequence is comprised by a host cell antibiotic resistance gene), for treating or preventing a disease or condition in a human or animal.

An aspect provides a method of treating or preventing a disease or condition in a human or animal subject, the method comprising administering a vector or medicament of the invention to the subject, wherein host cells comprised by a microbiome of the subject are modified by endogenous de-repressed Cas of the cells, and the treatment or prevention is carried out.

An aspect provides a method of killing a wild-type bacterial or archaeal cell (eg, E. coli or Salmonella cell), wherein the cell comprises an endogenous CRISPR/Cas system comprising nucleotide sequences encoding Cas3 and Cascade proteins, wherein Cas3 and/or Cascade is naturally repressed in the cell, the method comprising
(a) de-repressing said Cas3 and/or Cascade and
(b) introducing into the cell (i) a CRISPR array for production of one or more crRNAs in the cell; or (ii) one or more nucleotide sequences each encoding a respective guide RNA (gRNA, eg, a single guide RNA);
   wherein each crRNA or gRNA guides Cas or Cascade to modify a respective protospacer sequence of the host cell genome or to modify a protospacer sequence of an episome comprised by the host.

Optionally, in step (b) a vector according to the invention is introduced into the cell, thereby introducing (i) or (ii) into the cell.

Optionally, Cas3 transcription, expression or activity is de-repressed.

Optionally, Cas transcription, expression or activity is de-repressed, wherein the Cas is a Cascade Cas (eg, CasA, B, C, D or E).

Optionally, an expressible de-repressor sequence is introduced into the cell simultaneously or sequentially together with said array or gRNA-encoding sequence.

Optionally, the de-repressor sequence and the array or gRNA-encoding sequence are comprised by the same nucleic acid vector (eg, a phagemid, phage or plasmid).

Optionally, the de-repressor sequence and the array or gRNA-encoding sequence are comprised by the same operon or under the control of a common expression control (eg, same promoter) that is operable in the cell. This is advantageous to coordinate expression of these elements in the host cell.

Optionally, step (a) comprises expressing in the cell (i) LeuO or a, homologue, orthologue or functional equivalent thereof that is capable of forming a complex with H-NS; or (ii) a mutant of H-NS, StpA, LRP or CRP that is capable of forming a complex with H-NS, StpA, LRP or CRP repressor respectively.

A pharmaceutical composition, foodstuff, beverage, composition for environmental remediation, pesticide, herbicide, or cosmetic comprising a vector or vectors according to the invention are also provided.

An aspect provides a nucleic acid vector for introduction into a host cell, wherein the cell comprises a CRISPR/Cas system that is repressed by a repressor in the cell, the vector comprising
(a) A nucleotide sequence encoding a de-repressor that is capable of de-repressing the CRISPR/Cas system in the cell, wherein the sequence is expressible in the cell to produce the de-repressor; and
(b) A site for introduction of
   (i) a CRISPR array or a CRISPR spacer sequence for production of one or more crRNAs in the cell; or
   (ii) a nucleotide sequence encoding a guide RNA (gRNA, eg, a single guide RNA) in the cell;
      wherein said crRNA (eg, when comprised by a guide RNA) or gRNA is capable of guiding Cas to modify a respective protospacer sequence of the host cell genome or to modify (eg, cut or cause mutation of) a protospacer sequence of an episome comprised by the host in the presence of the de-repressor.

Optionally, site (b) is comprised by a CRISPR array, wherein the array is capable of accommodating one or more said spacer sequences for targeting respective protospacer sequences of the host cell. This is useful for pre-empting the development of resistance of the host cells to the vector.

Optionally, the array of (i) or sequence of (ii) is under the control of a promoter that is a strong and/or constitutive promoter for expression in the host cell. Strong promoters are useful to maximize the chances of expression during any or different phases of host cell population growth or existence.

Optionally, the sequence of (a) is under the control of a promoter that is a strong and/or constitutive promoter for expression of the de-repressor in the host cell.

Optionally, an expressible Cas3 sequence and/or an expressible htpG sequence for expression in the host cell. The upregulation of Cas3 by htpG may be advantageous to promote modification of the target sequence.

Optionally, each said crRNA is operable with a Cas (eg, a Cas nuclease, eg, Cas9 or Cas3) in the host cell.

Optionally, the Cas is encoded by an endogenous nucleotide sequence of the host cell genome, wherein when de-repressed the Cas has nuclease activity.

Optionally, the Cas is encoded by a nucleotide sequence comprised by the vector or by a different vector that can be introduced into the host cell for expression of said Cas therein.

Optionally, when the spacer of (i) or the sequence of (ii) has been inserted therein, the vector is then according to a vector of the invention that can be introduced into host cells.

The BglJ-RcsB heteromer is known to activate the HNS repressed leuO and bgl loci, and thus in an example, the vector(s) encode BglJ and/or RcsB, for formation of BglJ-RcsB heteromer in the host cell(s).

In an example, the protospacer is comprised by a gene encoding a protein that mediates host cell population quorum sensing, eg, a BglJ or LuxI family gene. LuxI family proteins generate N-acyl homoserine lactone (AHL) quorum sensing signals, and it may be beneficial to therefore target these to reduce host cell population growth, viability or proliferation.

In an example of a promoter herein, the promoter is a constitutive promoter that is devoid of a binding site of H-NS or a H-NS family member. A significant feature of constitutive promoters is the high level conservation of canonical TTGACA(-35)-17 bp-TATAAT(-10) sequence, and thus in an example, the promoter comprises a TTGACA and/or TATAAT sequence.

In an example of a promoter herein, the promoter is a H-NS promoter, eg, comprising the sequence of a H-NS promoter of the host cell species. This may be useful to provide for vector sequence expression at the same proportion and/or time as H-NS repressor in the cell(s).

Hha with H-NS increases the repressive ability of H-NS. In an example, the vector(s) encode an inhibitor Hha/H-NS multimerisation or YdgT/H-NS multimerisation, for expression of the inhibitor in the host cell(s).

In an example, the host cell(s) are S. enterica serovar Typhimurium strain LT2 cells. It is proposed that the expression of the type E (Cse) cas genes from S. enterica are likely to be regulated by H-NS and LeuO. For instance, in S. enterica serovar Typhi transcription of casA (STY3070) appears to be affected by H-NS and LeuO (Hernandez-Lucas et al., 2008), despite the poor conservation of the intergenic region between the divergently oriented cas3 and casA genes in this strain.

In an example, the host cell(s) are E. coli EPEC, EHEC, K12 or strain MG1655 cells.

Since H-NS is known to bind DNA of incoming phage or plasmid directly (Navarre et al., 2006; Navarre et al., 2007) this might result in redistribution of H-NS (Doyle et al., 2007; Dillon et al., 2010), allowing expression of the Cascade genes due to decreased local concentrations of the repressor. In an example, the vector(s) comprise a H-NS or StpA binding site. In an example, the vector(s) do not comprise a H-NS or StpA binding site.

As leuO expression is negatively regulated by H-NS and positively by LeuO itself (Hommais et al., 2001; Chen et al., 2005), this would further amplify the activating signal for cas gene transcription. In an example, the vector(s) comprise a plurality of LeuO-encoding sequences. This usefully may amplify the positive feedback in the presence of LeuO protein.

The intergenic region (IGLB, for intergenic region ygcL-ygcB) between the Cascade region, casA gene (ygcL) and the cas3 gene (ygcB) of the host cell may comprise a binding site for H-NS (eg, in E coli). Thus, in an example, the vector(s) encode an inhibitor that inhibits binding of the repressor to one or more IGLB region(s) of the host cell genome. For example in E coli, the following primers can be used to amplify a IGLB sequence—

UP-IGLB (Used as upstream primer for cloning of
the IGLB region)
(SEQ ID NO: 1)
5′-TTG TTC TCC TTC ATA TGC TCC GAC ATT TCT-3′
DOWN-IGLB (Used as downstream primer for cloning
of the IGLB region)
(SEQ ID NO: 2)
5′-CTT CGG GAA TGA TTG TTA TCA ATG ACG ATA-3′

The casA-cas3 intergenic region (here denoted IGLB) contains Pcas, for which H-NS has strong binding affinity as well as the divergently oriented anti-cas3 (known as anti-Pcas) promoter, that is located 80 bp upstream of Pcas and gives rise to an antisense transcript of unknown function (Pul et al, Mol Microbiol. 2010 March; 75(6):1495-512. doi: 10.1111/j.1365-2958.2010.07073.x. Epub 2010 Feb. 1, “Identification and characterization of E. coli CRISPR-cas promoters and their silencing by H-NS”). Both LeuO and H-NS bind the IGLB fragment, as determined by electrophoretic mobility shift assay (EMSA). In an example of the invention, the de-repressor and/or the repressor bind a CRISPR/Cas system IGLB comprised by the host cell genome. In an example of the invention, the de-repressor and/or the repressor bind a CRISPR/Cas system Pcas (or orthologue or homologue) comprised by the host cell genome. In an example of the invention, the de-repressor and/or the repressor bind a CRISPR/Cas system anti-Peas (or orthologue or homologue) comprised by the host cell genome. The binding site is comprised, for example, by a Type I CRISPR array of the host cell(s).

As an example test of a de-repressor, the de-repressor binds an IGLB sequence (which sequence is comprised by the host species genome), as determined by an electrophoretic mobility shift assay (EMSA) eg, as described in Westra et al 2010 (Westra et al, Mol Microbiol. 2010 September; 77(6):1380-93. doi: 10.1111/j.1365-2958.2010.07315.x. Epub 2010 Aug. 18, “H-NS-mediated repression of CRISPR-based immunity in Escherichia coli K12 can be relieved by the transcription activator LeuO”). Pre-bound LeuO will impede cooperative binding of H-NS to the IGLB fragment in an in vitro binding assay. In line with this, pre-bound H-NS is partly released from the IGLB when the de-repressor is added to the H-NS/LeuO complex. In order to map the binding region of LeuO or other de-repressor within the IGLB sequence, DNase I footprint analysis can be performed. Upon limited DNase I hydrolysis of the IGLB DNA, H-NS causes an extended footprint, as shown before (Pul et al., 2010).

The de-repressor may comprise an oligonucleotide which is either complementary to a binding site for the repressor (eg, H-NS) comprised by the host cell genome, eg, complementary to a Pcas promoter or anti-cas promoter of a CRISPR/Cas system of the host cell, wherein transcripts can be initiated from the Pcas promoter or the anti-cas promoter in the presence of the de-repressor. In another example, the de-repressor is not capable of binding LGLB DNA comprised by the host genome.

Optionally, the de-repressor is the dominant negative H-NS mutant protein G113D (Ueguchi et al., 1996; Pul et al., 2007). This H-NS mutant protein has lost DNA-binding activity,

but is able to form heteromers with wild-type H-NS. The resulting heteromers have also lost their DNA-binding properties (Pul et al., 2005). For example, the de-repressor is G113D or a functional equivalent thereof, eg, encoded in the vector(s) by a nucleotide sequence that is under the control of a strong and/or constitutive promoter for expression of G113D or the equivalent in the host cell(s).

In an example, the expression of the de-repressor in the host cell is inducible, eg, wherein the de-repressor is encoded by a vector nucleotide sequence that is under the control of an inducible promoter. For example, the induction can be physical (eg, heat induction), by light or by chemical means.

In an example, the method comprises or the vector is for de-repression of H-NS repression of RNA polymerase-promoter interaction in a CRISPR/Cas array in the host cell, wherein the Cas is said repressed Cas.

In an example, the repressor binds to a IGLB region of a CRISPR/Cas system of the host cell. In an example, the repressor binds to a promoter of a CRISPR/Cas system of the host cell. In an example, the repressor is an inhibitor of an RNA polymerase binding site of a CRISPR/Cas system of the host cell. In an example, the repressor is an inhibitor of an RNA transcription from a CRISPR/Cas system of the host cell. In an example, the repressor is an inhibitor of an RNA transcription from a transcription start site comprised by a CRISPR/Cas system of the host cell, wherein the transcription start site is within 100, 50 or 30 nucleotides upstream of the first (5′-most) nucleotide of the first CRISPR repeat of a CRISPR array of the system, or wherein the start site is within the leader region of a CRISPR array of the system. For example, the repressor is H-NH or StpA which bind such regions.

In the E. coli Type I-E system, the PAM corresponds to the 5′-AWG-3′ sequence located immediately upstream of (5′ of) a proto-spacer. Thus, when the host cell is an E coli cell, the protospacer is immediately 3′ of a PAM, wherein the PAM is 5′-AWG-3′, eg, AAG, AGG, GAG or ATG.

When the host cell is a S thermophilus cell, the protospacer is immediately 3′ or immediately 5′ of a PAM, wherein the PAM is NNAGAAW or NGGNG. In an example, the PAM is 5′-AW-3′, eg, AA or AT, or AG. Optionally, the PAM is a PAM of the CRISPR4 system of S thermophilus. For example, the CRISPR array of the invention or the guide RNA-encoding nucleotide sequence comprises a repeat sequence, wherein the repeat sequence is

(SEQ ID NO: 74)
5′-GTTTTTCCCGCACACGCGGGGGTGATCC-3′.

Optionally, the system comprises a repressed Cas and

(a) the Cas comprises the amino acid sequence selected from SEQ ID NO: 58, 60, 66 and 68, or an amino acid sequence that is at least 70, 75, 80, 85, 90, 95, 96, 97, 98 or 99% (eg, at least 80%) identical to the selected sequence, or is an orthologue or homologue thereof that is operable with a repeat comprising a sequence selected from SEQ ID NOs: 49-52 and a PAM comprising or consisting of AWG, eg, AAG, AGG, GAG or ATG, wherein optionally the host cell is an E coli cell; or
(b) the Cas comprises the amino acid sequence selected from SEQ ID NO: 56, 64, 70 and 72, or an amino acid sequence that is at least 70, 75, 80, 85, 90, 95, 96, 97, 98 or 99% (eg, at least 80%) identical to the selected sequence, or is an orthologue or homologue thereof that is operable with a repeat comprising SEQ ID NO: 53, wherein optionally the host cell is a S enterica; or
(c) the Cas comprises the amino acid sequence selected from SEQ ID NO:62, or an amino acid sequence that is at least 70, 75, 80, 85, 90, 95, 96, 97, 98 or 99% (eg, at least 80%) identical to the selected sequence, or is an orthologue or homologue thereof that is operable with a PAM comprising or consisting of NNAGAAW, NGGNG or AW, eg, AA, AT or AG, wherein optionally the host cell is a S thermophilus cell.

Optionally, the system comprises a repressed Cas and the Cas is operable with

(a) a repeat comprising a sequence selected from SEQ ID NOs: 49-52 and a PAM comprising or consisting of AWG, eg, AAG, AGG, GAG or ATG, wherein optionally the host cell is an E coli cell;
(b) a repeat comprising SEQ ID NO: 53, wherein the host cell is a S enterica; or
(c) a PAM comprising or consisting of NNAGAAW, NGGNG or AW, eg, AA, AT or AG, wherein optionally the host cell is a S thermophilus cell.

Optionally, the system comprises a repressed Cas and the Cas is operable with

(a) a PAM comprising or consisting of AWG, eg, AAG, AGG, GAG or ATG and optionally the Cas nuclease comprises the amino acid sequence selected from SEQ ID NO: 58, 60, 66 and 68, or an amino acid sequence that is at least 70, 80, 90, 95 or 98% identical to the selected sequence, wherein optionally the host cell is an E coli cell; or
(b) a PAM comprising or consisting of NNAGAAW, NGGNG or AW, eg, AA, AT or AG and optionally the Cas nuclease comprises the amino acid sequence selected from SEQ ID NO: 62, or an amino acid sequence that is at least 70, 80, 90, 95 or 98% identical to the selected sequence, wherein optionally the host cell is a S thermophilus cell.

Optionally, the target nucleotide sequence or protospacer comprises the sequence of at least 5, 6, 7, 8, 9 or 10 contiguous nucleotides immediately 3′ of a said PAM in the genome of the host cell. Optionally, the target nucleotide sequence or protospacer comprises the sequence of at least 5, 6, 7, 8, 9 or 10 contiguous nucleotides immediately 5′ of a said PAM in the genome of the host cell. In an example, the PAM is comprised by a chromosome or episome of the host cell

Optionally, the repressor is
(i) H-NS comprising an amino acid sequence selected from SEQ ID NO: 17, 19, 21, 23, 25 and 27, or an amino acid sequence that is at least 70, 80, 90, 95 or 98% identical to said selected sequence; or
(ii) StpA comprising an amino acid sequence selected from SEQ ID NO: 29, 31, 33 and 35, or an amino acid sequence that is at least 70, 80, 90, 95 or 98% identical to said selected sequence.

Optionally, the de-repressor is
(iii) LeuO comprising an amino acid sequence selected from SEQ ID NO: 3, 5, 7, 9, 11, 13 and 15, or an amino acid sequence that is at least 70, 80, 90, 95 or 98% identical to said selected sequence; or
(iv) LRP comprising an amino acid sequence selected from SEQ ID NO: 37, 39 and 41, or an amino acid sequence that is at least 70, 80, 90, 95 or 98% identical to said selected sequence; or
(v) CRP comprising an amino acid sequence selected from SEQ ID NO: 43, 45 and 47, or an amino acid sequence that is at least 70, 80, 90, 95 or 98% identical to said selected sequence.

In an example, the CRISPR/Cas system comprised by the host cell (eg, a mammalian, human, mouse, bacterial or archaeal cell) comprises a repressed Cas, eg, a Cast, 2, 3, 9, A, B, C, D or E. For example, the Cas is encoded by an endogenous nucleotide sequence of the host cell, wherein the host cell is a bacterial or archaeal cell. In another example, the Cas is encoded by an exogenous nucleotide sequence comprised by the cell, eg, that has been introduced by a vector (eg, a vector of the invention).

In certain embodiments of the invention, the host cell is a bacterial or archaeal cell that comprises an endogenous CRISPR/Cas system, wherein the system comprises a repressed Cascade or Cas (eg, a Cas1, 2, 3, 9, A, B, C, D or E) encoded by one or more endogenous nucleotide sequences of the host genome. Advantageously, the invention may be used to de-repress the Cascade or Cas, whereby the Cascade or Cas can be harnessed to modify (eg, cut) a target protospacer sequence comprised by the host genome (eg, a chromosomal or episomal protospacer sequence), for example to kill the cell. This aspect involves introducing one or more vectors into the host cell encoding a crRNA or guide RNA (eg, single guide RNA) that is operable with the Cascade or Cas once it has been de-repressed. De-repression is carried out by means of the de-repressor of the invention carried by one or more of said vectors, wherein the de-repressor is expressed inside the host cell for de-repressing the repressed Cascade or Cas. Usefully, the ability to harness a de-repressed endogenous Cas enables one to omit corresponding Cas-encoding sequence(s) on the vector(s) of the invention. This frees up valuable space on the vector (especially considering that some Cas-encoding sequences are large, eg, S pyogenes Cas9 sequence is 4.2 kb, which nears the packaging capacity of a phage vector for example. The free space enables one to include more spacers and/or CRISPR arrays or gRNA-encoding sequences to enable multiplexing of cutting of host sequences. This is useful to minimize the chances of the host evolving resistance to vector(s) of the invention. Another advantage of being to harness endogenous Cas, rather than relying on an exogenous Cas encoded by a vector, is that the endogenous Cas is native to the host cell machinery and thus is likely to work efficiently once de-repressed. An exogenous Cas, eg, a S pyogenes Cas expressed in an E coli cell, may be inferior as it is a foreign protein and may not work so efficiently with the E coli machinery. Thus, optionally, the vector(s) of the invention are devoid of a nucleotide sequence encoding the repressed Cas or Cascade; or the vector(s) are devoid of any nucleotide sequence encoding a Cas. Optionally, the method of the invention does not comprise the introduction into the host cell(s) of a nucleotide sequence encoding the repressed Cas, or does not comprise the introduction into the host cell(s) of any nucleotide sequence encoding a Cas.

An example application of this configuration of the invention is the introduction of one or more vectors of the invention into an E coli cell (eg, an Escherichia coli O157 H7 EDL933 (EHEC) cell) that comprises a repressed Cas3 and/or Cascade (or a CasA thereof) (wherein H-NS represses the Cas and/or Cascade). The vector(s) comprise (a) a nucleotide sequence encoding a de-repressor (such as LeuO) that is capable of de-repressing the Cascade or Cas in the cell, wherein the sequence is expressible in the cell to produce the de-repressor; and (b) a CRISPR array for production of one or more crRNAs in the cell; and/or one or more nucleotide sequences encoding a respective guide RNA (gRNA, eg, a single guide RNA) in the cell; wherein each crRNA or gRNA is capable of guiding the Cas or a Cas of the Cascade to modify a respective protospacer sequence of the host cell genome or to modify a protospacer sequence of an episome comprised by the host in the presence of the de-repressor. Component (b) comprises a CRISPR repeat sequence that is operable with the de-repressed Cas or Cascade, eg, the repeat sequence comprises or consists of a sequence selected from SEQ ID NO: 49-52. In an example, the de-repressed Cas is a Cas3 comprising an amino acid sequence of SEQ ID NO: 58 or 60. In an example, the de-repressed Cas is a CasA comprising an amino acid sequence of SEQ ID NO: 66 or 68. Optionally, the target nucleotide sequence or protospacer comprises the sequence of at least 5, 6, 7, 8, 9 or 10 contiguous nucleotides immediately 3′ of a PAM in the genome of the host cell, wherein the PAM is selected from AWG, AAG, AGG, GAG and ATG.

An example application of this configuration of the invention is the introduction of one or more vectors of the invention into an S enterica cell (eg, a Salmonella enterica subsp. enterica serovar Typhimurium cell, eg, Salmonella enterica subsp. enterica serovar Typhimurium LT2 cell or Salmonella enterica subsp. enterica serovar Typhimurium Paratyphi A cell) that comprises a repressed Cas3 and/or Cascade (or a CasA thereof) (wherein H-NS represses the Cas and/or Cascade). The vector(s) comprise (a) a nucleotide sequence encoding a de-repressor (such as LeuO) that is capable of de-repressing the Cascade or Cas in the cell, wherein the sequence is expressible in the cell to produce the de-repressor; and (b) a CRISPR array for production of one or more crRNAs in the cell; and/or one or more nucleotide sequences encoding a respective guide RNA (gRNA, eg, a single guide RNA) in the cell; wherein each crRNA or gRNA is capable of guiding the Cas or a Cas of the Cascade to modify a respective protospacer sequence of the host cell genome or to modify a protospacer sequence of an episome comprised by the host in the presence of the de-repressor. Component (b) comprises a CRISPR repeat sequence that is operable with the de-repressed Cas or Cascade, eg, the repeat sequence comprises or consists of SEQ ID NO: 53. In an example, the de-repressed Cas is a Cas3 comprising an amino acid sequence of SEQ ID NO: 56 or 64. In an example, the de-repressed Cas is a CasA comprising an amino acid sequence of SEQ ID NO: 70 or 72. Optionally, the target nucleotide sequence or protospacer comprises the sequence of at least 5, 6, 7, 8, 9 or 10 contiguous nucleotides immediately 3′ of a PAM in the genome of the host cell, wherein the PAM is operable with the Cas3.

Optionally, the vector is devoid of a Cas-encoding nucleotide sequence or a nucleotide sequence encoding a repressed Cas of the system.

Optionally, in an alternative the vector comprises component (a) but not component (b), wherein component (b) (and optionally also component (b)) is comprised by a second vector that is in combination with the first vector; wherein optionally the vectors are devoid of a Cas-encoding nucleotide sequence or a nucleotide sequence encoding a repressed Cas of the system.

Optionally, the system comprises a repressed Cascade (eg, CasA, B, C, D and E) and the de-repressor is capable of de-repressing the Cascade in the host cell (eg, an E coli or Salmonella cell), optionally wherein each vector is devoid of a nucleotide sequence encoding one or more Cas of said repressed Cascade.

Optionally, the system comprises a repressed CasA, Cas3 or Cas9 and the de-repressor is capable of de-repressing the Cas in the host cell (eg, an E coli or Salmonella cell), optionally wherein each vector is devoid of a nucleotide sequence encoding the Cas.

### Aspects:

Certain Aspects of the invention are as follows:—
1. A nucleic acid vector for introduction into a host cell, wherein the cell comprises a CRISPR/Cas system that is repressed by a repressor in the cell, the vector comprising
   (a) A nucleotide sequence encoding a de-repressor that is capable of de-repressing the CRISPR/Cas system in the cell, wherein the sequence is expressible in the cell to produce the de-repressor; and
   (b) A CRISPR array for production of one or more crRNAs in the cell; and/or one or more nucleotide sequences encoding a respective guide RNA (gRNA, eg, a single guide RNA) in the cell; wherein each crRNA or gRNA is capable of guiding Cas to modify a respective protospacer sequence of the host cell genome or to modify a protospacer sequence of an episome comprised by the host in the presence of the de-repressor.
2. The vector of Aspect 1, wherein transcription of one or more components of the CRISPR/Cas system is repressed.
3. The vector of Aspect 1 or 2, wherein transcription of one or more Cas sequences is repressed.
4. The vector of any preceding Aspect, wherein transcription of one or more of CasA, B, C, D and E of a Type I CRISPR/Cas system is repressed.
5. The vector of any preceding Aspect, wherein transcription of a Cas3 is repressed.
6. The vector of any preceding Aspect, wherein Cas modification of the host cell genome
   (a) kills the host cell;
   (b) reduces growth or proliferation of the cell or episome;
   (c) increases growth or proliferation of the cell or episome;
   (d) reduces or prevents transcription of a nucleotide sequence that comprises or is adjacent a said protospacer sequence; or
   (e) increases transcription of a nucleotide sequence that comprises or is adjacent a said protospacer sequence.
7. The vector of any preceding Aspect, wherein components (a) and (b) are instead comprised by first and second vectors that are different, for introduction of the vectors into the host cell wherein said Cas modification takes place.
8. The vector of any preceding Aspect, wherein the de-repressor is a protein or an RNA.
9. The vector of any preceding Aspect, wherein the repressor is H-NS, StpA, LRP or CRP.
10. The vector of any preceding Aspect, wherein the de-repressor is a mutant H-NS, StpA, LRP or CRP that is capable of forming a complex with H-NS, StpA, LRP or CRP repressor respectively in the host cell to prevent or reduce repression of the CRISPR/Cas system.
11. The vector of any preceding Aspect, wherein the repressor is H-NS or StpA and the de-repressor is LeuO.
12. The vector of any preceding Aspect, wherein the episome is a plasmid.
13. The vector of any preceding Aspect, wherein the cell is a bacterial or archaeal cell.
14. The vector of any preceding Aspect, wherein nucleotide sequence (a) comprises a constitutive promoter or strong promoter for expression of the sequence in the host cell.
15. The vector of any preceding Aspect, wherein nucleotide sequence or array (b) comprises a constitutive promoter or strong promoter for expression of the sequence or array in the host cell.
16. The vector of any preceding Aspect, wherein (a) and (b) are comprised by the same operon or under the control of a common expression control (eg, same promoter) that is operable in the cell.
17. The vector of Aspect 16, wherein the promoter is a strong and/or constitutive promoter for expression in the host cell.
18. The vector of any preceding Aspect, wherein the host cell is a wild-type host cell.
19. The vector of any preceding Aspect, wherein the vector comprises an expressible htpG sequence.
20. The vector of any preceding Aspect, wherein the cell comprises a CRISPR/Cas system comprising Cascade and Cas3, wherein the Cascade is repressed (eg, by H-NS) in the host cell, wherein the vector comprises
   (i) An expressible nucleotide sequence encoding a de-repressor (eg, LeuO) of said Cascade repression; and
   (ii) An expressible nucleotide sequence encoding a Cas3, wherein the Cas 3 is capable of functioning with de-repressed Cascade in the host cell; Wherein the nucleotide sequences are capable of being expressed in the host cell.
21. The vector of Aspect 20, wherein the nucleotide sequences of (i) and (ii) are comprised by the same operon or under the control of a common expression control (eg, same promoter) that is operable in the cell.
22. The vector of Aspect 20 or 21, wherein sequences (i) and (ii) and the CRISPR array or sequence encoding said gRNA are comprised by two or more different vectors for introduction into the host cell for expression of the de-repressor, Cas3 and array or gRNA together in the host cell.
23. A nucleic acid vector (optionally according to any preceding Aspect) for introduction into a bacterial or archaeal host cell, wherein the cell comprises an endogenous CRISPR/Cas system that is naturally repressed by a repressor in the cell, the vector comprising
   (a) A nucleotide sequence encoding a de-repressor that is capable of de-repressing the CRISPR/Cas system in the cell, wherein the sequence is expressible in the cell to produce the de-repressor; and
   (b) A CRISPR array for production of one or more crRNAs in the cell; and/or one or more nucleotide sequences encoding a respective guide RNA (gRNA, eg, a single guide RNA) in the cell; wherein each crRNA or gRNA is capable of guiding Cas to modify a protospacer sequence of the host cell genome or to modify a protospacer sequence of an episome comprised by the host in the presence of the de-repressor;
      Wherein the repressor is H-NS, StpA, LRP or CRP or a functional equivalent thereof encoded by the cell genome; and
      Wherein the de-repressor is LeuO or a functional equivalent thereof that is capable of forming a complex with H-NS; or a mutant of H-NS, StpA, LRP or CRP that is capable of forming a complex with H-NS, StpA, LRP or CRP repressor respectively.
24. The vector of Aspect 23, wherein the cell is an E coli (eg, E coli K12) or Salmonella (eg, S enteric serovar typhimurium) cell.
25. The vector of Aspect 23 or 24, wherein the Cas is Cas3.
26. The vector of any one of Aspect 23 to 25, wherein the CRISPR/Cas system is a Type I (eg, Type I-E or Type I-F) system.
27. The vector of any one of Aspects 23 to 26, wherein the protospacer sequence is comprised by an essential gene, virulence gene or antibiotic resistance gene comprised by the cell.
28. The vector of any one of Aspects 23 to 27, wherein the vector comprises no sequences from the group consisting of CasA, B, C, D and E nucleotide sequences, or wherein the vector does not comprise all of the sequences of said group.
29. The vector of any one of Aspects 23 to 28, wherein the vector comprises no sequences from the group consisting of Cas1, Cas2, Cas5 and Cas6 sequences.
30. The vector of any one of Aspects 23 to 29, wherein the vector comprises no Cas 3 nucleotide sequence.
31. The vector of any preceding Aspect for medical use for treating or preventing a disease or condition in a human or animal subject, wherein the host cell is comprised by the subject.
32. The vector of any preceding Aspect for killing said host cell or for reducing the growth or proliferation thereof in a human, animal or plant microbiome.
33. A plurality of vectors of any preceding Aspect for killing a plurality of host cells or for reducing the growth or proliferation thereof.
34. The vector(s) of any preceding Aspect, wherein the or each host cell is comprised by a microbiome (eg, gut microbiome or environmental microbiome) comprising a plurality of said host cells and comprising one or more cells of a species or strain that is different from the species or strain of the host cells.
35. A medicament comprising a plurality of vectors according to any preceding Aspect, optionally further comprising one or more medical drugs (eg, an anti-cancer medicament) or antibiotics (eg, wherein the protospacer sequence is comprised by a host cell antibiotic resistance gene), for treating or preventing a disease or condition in a human or animal.
36. A method of treating or preventing a disease or condition in a human or animal subject, the method comprising administering a vector or medicament of any preceding Aspect to the subject, wherein host cells comprised by a microbiome of the subject are modified by endogenous de-repressed Cas of the cells, and the treatment or prevention is carried out.
37. A method of killing a wild-type bacterial or archaeal cell (eg, E coli or Salmonella cell), wherein the cell comprises an endogenous CRISPR/Cas system comprising nucleotide sequences encoding Cas3 and Cascade proteins, wherein Cas3 and/or Cascade is naturally repressed in the cell, the method comprising
   (a) de-repressing said Cas3 and/or Cascade and
   (b) introducing into the cell (i) a CRISPR array for production of one or more crRNAs in the cell; or (ii) one or more nucleotide sequences encoding a respective guide RNA (gRNA, eg, a single guide RNA);
      wherein each crRNA or gRNA guides Cas or Cascade to modify a respective protospacer sequence of the host cell genome or to modify a protospacer sequence of an episome comprised by the host.
38. The method of Aspect 37, wherein in step (b) a vector according to any one of Aspects 1 to 33 is introduced into the cell.
39. The method of Aspect 37 or 38, wherein Cas3 transcription, expression or activity is de-repressed.
40. The method of Aspect 37, 38 or 39, wherein Cas transcription, expression or activity is de-repressed, wherein the Cas is a Cascade Cas (eg, CasA, B, C, D or E).
41. The method of any one of Aspects 37 to 40, wherein an expressible de-repressor sequence is introduced into the cell simultaneously or sequentially together with said array or gRNA-encoding sequence.
42. The method of any one of Aspects 37 to 41, wherein the de-repressor sequence and the array or gRNA-encoding sequence are comprised by the same nucleic acid vector (eg, a phagemid, phage or plasmid).
43. The method of any one of Aspects 37 to 42, wherein the de-repressor sequence and the array or gRNA-encoding sequence are comprised by the same operon or under the control of a common expression control (eg, same promoter) that is operable in the cell.
44. The method of any one of Aspects 37 to 43, wherein step (a) comprises expressing in the cell (i) LeuO or a functional equivalent thereof that is capable of forming a complex with H-NS; or (ii) a mutant of H-NS, StpA, LRP or CRP that is capable of forming a complex with H-NS, StpA, LRP or CRP repressor respectively.
45. A pharmaceutical composition, foodstuff, beverage, composition for environmental remediation, pesticide, herbicide, or cosmetic comprising a vector or vectors according to any one of Aspects 1 to 34.
46. A nucleic acid vector for introduction into a host cell, wherein the cell comprises a CRISPR/Cas system that is repressed by a repressor in the cell, the vector comprising
   (a) A nucleotide sequence encoding a de-repressor that is capable of de-repressing the CRISPR/Cas system in the cell, wherein the sequence is expressible in the cell to produce the de-repressor; and
   (b) A site for introduction of
      (i) a CRISPR array or a CRISPR spacer sequence for production of one or more crRNAs in the cell;
      (ii) a nucleotide sequence encoding a guide RNA (gRNA, eg, a single guide RNA) in the cell;
         wherein said crRNA or gRNA is capable of guiding Cas to modify a respective protospacer sequence of the host cell genome or to modify a protospacer sequence of an episome comprised by the host in the presence of the de-repressor.
47. The vector of Aspect 46, wherein site (b) is comprised by a CRISPR array, wherein the array is capable of accommodating one or more said spacer sequences for targeting respective protospacer sequences of the host cell.
48. The vector of Aspect 46 or 47, wherein the array of (i) or sequence of (ii) is under the control of a promoter that is a strong and/or constitutive promoter for expression in the host cell.
49. The vector of Aspect 46 or 47, wherein the sequence of (a) is under the control of a promoter that is a strong and/or constitutive promoter for expression of the de-repressor in the host cell.
50. The vector of any one of Aspects 46 to 49, comprising an expressible Cas3 sequence and/or an expressible htpG sequence for expression in the host cell.
51. The vector or any one of Aspects 46 to 50, wherein each said crRNA is operable with a Cas (eg, a Cas nuclease) in the host cell.
52. The vector of Aspect 51, wherein the Cas is encoded by an endogenous nucleotide sequence of the host cell genome.
53. The vector of Aspect 51, wherein the Cas is encoded by a nucleotide sequence comprised by the vector or by a different vector that can be introduced into the host cell for expression of said Cas therein.
54. The vector of any one of Aspects 46 to 53, wherein when the spacer of (i) or the sequence of (ii) has been inserted therein, the vector is then according to a vector of any one of Aspects 1 to 34.
55. A method of treating or preventing a disease or condition in a human or animal subject, the method comprising administering a vector to the subject, wherein bacterial or archaeal host cells (eg, E coli, Salmonella, or S enteric serovar typhimurium) comprised by a microbiome of the subject are modified by endogenous de-repressed Cas of the cells, and the treatment or prevention is carried out; wherein
   (i) each cell comprises a CRISPR/Cas system that is repressed by a repressor in the cell,
   (ii) the vector comprising an nucleotide sequence encoding a de-repressor that is capable of de-repressing the CRISPR/Cas system in the cell, wherein the sequence is expressible in the cell to produce the de-repressor; and
   (iii) the vector being devoid of a CRISPR array for production of one or more crRNAs in the cell; and devoid of one or more nucleotide sequences encoding a respective guide RNA (gRNA, eg, a single guide RNA) in the cell.
56. A medicament comprising a plurality of nucleic acid vectors for introduction into bacterial or archaeal host cells, optionally further comprising one or more medical drugs (eg, an anti-cancer medicament) or antibiotics (eg, wherein the protospacer sequence is comprised by a host cell antibiotic resistance gene), for treating or preventing a disease or condition in a human or animal; wherein
   (i) each cell comprises a CRISPR/Cas system that is repressed by a repressor selected from H-NS and/or StpA in the cell,
   (ii) the vector comprising an nucleotide sequence encoding a de-repressor that is capable of de-repressing the CRISPR/Cas system in the cell, wherein the sequence is expressible in the cell to produce the de-repressor optionally under the control of a strong and/or constitutive promoter; and
   the vector being devoid of a CRISPR array for production of one or more crRNAs in the cell; and devoid of one or more nucleotide sequences encoding a respective guide RNA (gRNA, eg, a single guide RNA) in the cell.
57. The method or Aspect 55 or medicament of Aspect 56, wherein a Cascade Cas, Cas3 or Cas9 is repressed.
58. The vector, medicament, method or composition of any preceding Aspect, wherein the system comprises a repressed Cas and
   (a) the Cas comprises the amino acid sequence selected from SEQ ID NO: 58, 60, 66 and 68, or an amino acid sequence that is at least 70, 75, 80, 85, 90, 95, 96, 97, 98 or 99% (eg, at least 80%) identical to the selected sequence, or is an orthologue or homologue thereof that is operable with a repeat comprising a sequence selected from SEQ ID NOs: 49-52 and a PAM comprising or consisting of AWG, eg, AAG, AGG, GAG or ATG, wherein optionally the host cell is an E coli cell; or
   (b) the Cas comprises the amino acid sequence selected from SEQ ID NO: 56, 64, 70 and 72, or an amino acid sequence that is at least 70, 75, 80, 85, 90, 95, 96, 97, 98 or 99% (eg, at least 80%) identical to the selected sequence, or is an orthologue or homologue thereof that is operable with a repeat comprising SEQ ID NO: 53, wherein optionally the host cell is a S enterica; or
   (c) the Cas comprises the amino acid sequence selected from SEQ ID NO:62, or an amino acid sequence that is at least 70, 75, 80, 85, 90, 95, 96, 97, 98 or 99% (eg, at least 80%) identical to the selected sequence, or is an orthologue or homologue thereof that is operable with a PAM comprising or consisting of NNAGAAW, NGGNG or AW, eg, AA, AT or AG, wherein optionally the host cell is a S thermophilus cell.
59. The vector, medicament, method or composition of any preceding Aspect, wherein the system comprises a repressed Cas and the Cas is operable with
   (a) a repeat comprising a sequence selected from SEQ ID NOs: 49-52 and a PAM comprising or consisting of AWG, eg, AAG, AGG, GAG or ATG, wherein optionally the host cell is an E coli cell;
   (b) a repeat comprising SEQ ID NO: 53, wherein the host cell is a S enterica; or
   (c) a PAM comprising or consisting of NNAGAAW, NGGNG or AW, eg, AA, AT or AG, wherein optionally the host cell is a S thermophilus cell.
60. The vector, medicament, method or composition of any preceding Aspect, wherein the system comprises a repressed Cas and the Cas is operable with
   (a) a PAM comprising or consisting of AWG, eg, AAG, AGG, GAG or ATG and optionally the Cas nuclease comprises the amino acid sequence selected from SEQ ID NO: 58, 60, 66 and 68, or an amino acid sequence that is at least 70, 75, 80, 85, 90, 95, 96, 97, 98 or 99% (eg, at least 80%) identical to the selected sequence, wherein optionally the host cell is an E coli cell; or
   (b) a PAM comprising or consisting of NNAGAAW, NGGNG or AW, eg, AA, AT or AG and optionally the Cas nuclease comprises the amino acid sequence selected from SEQ ID NO: 62, or an amino acid sequence that is at least 70, 75, 80, 85, 90, 95, 96, 97, 98 or 99% (eg, at least 80%) identical to the selected sequence, wherein optionally the host cell is a S thermophilus cell.
61. The vector, medicament, method or composition of any one of Aspects 58 to 60, wherein the protospacer comprises the sequence of at least 5, 6, 7, 8, 9 or 10 contiguous nucleotides immediately 3′ of a said PAM in the genome of the host cell.
62. The vector, medicament, method or composition of any one of Aspects 58 to 60, wherein the protospacer comprises the sequence of at least 5, 6, 7, 8, 9 or 10 contiguous nucleotides immediately 5′ of a said PAM in the genome of the host cell.
63. The vector, medicament, method or composition of any preceding Aspect, wherein the repressor is
   (i) H-NS comprising an amino acid sequence selected from SEQ ID NO: 17, 19, 21, 23, 25 and 27, or an amino acid sequence that is at least 70, 75, 80, 85, 90, 95, 96, 97, 98 or 99% (eg, at least 80%) identical to said selected sequence; or
   (ii) StpA comprising an amino acid sequence selected from SEQ ID NO: 29, 31, 33 and 35, or an amino acid sequence that is at least 70, 75, 80, 85, 90, 95, 96, 97, 98 or 99% (eg, at least 80%) identical to said selected sequence.
64. The vector, medicament, method or composition of any preceding Aspect, wherein the de-repressor is
   (iii) LeuO comprising an amino acid sequence selected from SEQ ID NO: 3, 5, 7, 9, 11, 13 and 15, or an amino acid sequence that is at least 70, 75, 80, 85, 90, 95, 96, 97, 98 or 99% (eg, at least 80%) identical to said selected sequence; or
   (iv) LRP comprising an amino acid sequence selected from SEQ ID NO: 37, 39 and 41, or an amino acid sequence that is at least 70, 75, 80, 85, 90, 95, 96, 97, 98 or 99% (eg, at least 80%) identical to said selected sequence; or
   (v) CRP comprising an amino acid sequence selected from SEQ ID NO: 43, 45 and 47, or an amino acid sequence that is at least 70, 75, 80, 85, 90, 95, 96, 97, 98 or 99% (eg, at least 80%) identical to said selected sequence.
65. The vector, medicament, method or composition of any preceding Aspect, wherein the vector is devoid of a Cas-encoding nucleotide sequence or a nucleotide sequence encoding a repressed Cas of the system.
66. The vector, medicament, method or composition of any preceding Aspect, wherein alternatively the vector comprises component (a) but not component (b), wherein component (b) is comprised by a second vector that is in combination with the first vector; wherein optionally the vectors are devoid of a Cas-encoding nucleotide sequence or a nucleotide sequence encoding a repressed Cas of the system.
67. The vector, medicament, method or composition of any preceding Aspect, wherein the system comprises a repressed Cascade (eg, CasA, B, C, D and E) and the de-repressor is capable of de-repressing the Cascade in the host cell (eg, an E coli or Salmonella cell), optionally wherein each vector is devoid of a nucleotide sequence encoding one or more Cas of said repressed Cascade.
68. The vector, medicament, method or composition of any preceding Aspect, wherein the system comprises a repressed CasA, Cas3 or Cas9 and the de-repressor is capable of de-repressing the Cas in the host cell (eg, an E coli or Salmonella cell), optionally wherein each vector is devoid of a nucleotide sequence encoding the Cas.

### Recombineering

The invention also provides a nucleic acid recombineering method as follows:—
69. An in vitro method of carrying out nucleic acid (eg, DNA) recombineering in a cell (eg, a bacterial cell, eg an E coli cell), wherein the cell comprises a CRISPR/Cas system that is repressed by a repressor, the method comprising
   (a) Introducing a nucleic acid of interest (NOI) into the cell (eg, by electroporation);
   (b) Introducing a vector of the invention (eg, according to any one of the above Aspects or Clauses below) into the cell (eg, by electroporation), wherein steps (a) and (b) are carried out simultaneously or in any order;
   (c) Expressing in the cell the de-represssor and the crRNA or gRNA encoded by the vector,
   wherein the de-repressor de-represses the CRISPR/Cas system and a Cas nuclease of the system is guided by the crRNA or gRNA to modify (eg, cut) a protospacer sequence comprised by the NOI; and
   (d) Optionally isolating the modified NOI.

The cell is a recombineering-competent cell, eg, comprising rac prophage RecE/RecT or lambda Redαβδ. For example, the cell is a recombineering-competent cell comprising a lambda red recombination system. In an example, the NOI is a DNA (eg, a dsDNA or a ssDNA). In another example, the NOI is a RNA. The isolated modified NOI may comprise modifications in addition to the modification produced by the Cas, for example, modifications made before or after the modification made by the Cas.

This aspect of the invention is useful for controlling the recombineering method. For example, the initiation, timing and/or duration of Cas modification (eg, Cas cutting) can be controlled by expression of the de-repressor. For example, the NOI and a template nucleic acid or insert nucleic acid or other component of the recombineering method, may be introduced first into the cell, followed by expression of the de-repressor, whereby modification is carried out by the Cas and the template/insert nucleic acid is used to also modify or copy the NOI modified by the Cas. For example, the method comprises introducing a second NOI into the cell simultaneously or sequentially with the introduction of the first NOI; the de-repressor is expressed; the Cas cuts the protospacer, thereby producing recombinogenic ends in the first NOI; nucleotide sequence comprised by the second NOI is inserted at or adjacent the cut in the first NOI; and optionally a contiguous modified NOI is produced comprising sequence of the first NOI contiguous with sequence of the second NOI. In another embodiment, the method comprises using the second NOI to retrieve a sequence of the first NOI that is at or adjacent the cut. See, for example, WO2017/118598 for suitable techniques of inserting or retrieving sequences that can be used in the method of the invention and which are thus incorporated herein by reference.
70. The method of Aspect 69, wherein the nuclease is a dsDNA nuclease.
71. The method of Aspect 69, wherein the nuclease is a ssDNA nuclease.
72. The method of Aspect 69, wherein the nuclease is a nickase.
73. The method of any one of Aspects 69 to 72, wherein the nuclease is a Cas9.
74. The method of any one of Aspects 69 to 72, wherein the nuclease is a Cas3 and optionally the repressor represses Cascade (eg, CasA).
75. The method of any one of Aspects 69 to 74, comprising introducing the isolated modified NOI into a second cell (eg, a non-human vertebrate, mammalian, human, animal (eg, cow, pig sheep, goat, livestock, fish, salmon or horse), rodent, mouse, rat or zebrafish or Xenopus cell) and optionally obtaining progeny cells therefrom.
76. The method of Aspect 75, wherein the second cell is an embryonic stem cell (ES cell) or induced pluripotent stem cell (iPS).
   For example the second cell is a non-human animal (eg, mammal or non-human vertebrate) cell, such as a rodent, mouse or rat cell.
77. The method of Aspect 76, comprising developing the second cell or a progeny cell into a non-human animal (eg, a cow, pig sheep, goat, livestock, fish, salmon, horse, rodent, mouse, rat, zebrafish or Xenopus).
78. The method of Aspect 77, further comprising isolating a protein or a nucleic acid (or a nucleotide sequence thereof) from the animal, eg, isolating an antibody, antibody chain, antibody variable region or nucleic acid thereof.
79. The method of Aspect 78, further comprising inserting the nucleic acid (or a nucleotide sequence thereof) into an expression vector or a host cell for expression of a protein comprising an amino acid sequence (eg, an antibody variable domain) encoded by the nucleic acid or nucleotide sequence thereof, expressing the protein and isolating the protein, and optionally formulating the isolated protein into a medicament for use in humans or animals.

Optionally the nucleic acid or the sequence is mutated or fused to another nucleic acid or nucleotide sequence before, during or after insertion into the expression vector. For example, an antibody variable domain sequence can be operatively connected in the vector to a nucleotide sequence encoding an antibody constant region for expression of antibody chains from the vector.

### Clauses

Certain Clauses of the invention are as follows:—
1. A nucleic acid vector for introduction into a host cell, wherein the cell comprises a CRISPR/Cas system that is repressed by a repressor in the cell, the vector comprising
   (a) A nucleotide sequence encoding a de-repressor that is capable of de-repressing the CRISPR/Cas system in the cell, wherein the sequence is expressible in the cell to produce the de-repressor; and
   (b) A CRISPR array for production of one or more crRNAs in the cell; and/or one or more nucleotide sequences encoding a respective guide RNA (gRNA) in the cell; wherein each cRNA or gRNA is capable of guiding Cas to modify a respective protospacer sequence of the host cell genome or to modify a protospacer sequence of an episome comprised by the host in the presence of the de-repressor.
2. The vector of Clause 1, wherein nucleotide sequence (a) comprises a constitutive promoter or strong promoter for expression of the sequence in the host cell.
3. The vector of Clause 1 or 2, wherein nucleotide sequence or array (b) comprises a constitutive promoter or strong promoter for expression of the sequence or array in the host cell.
4. The vector of any preceding Clause, wherein (a) and (b) are comprised by the same operon or under the control of a common promoter that is operable in the cell.
5. The vector of any preceding Clause, wherein the host cell is a wild-type host cell.
6. The vector of any preceding Clause, wherein transcription of one or more Cas sequences is repressed, optionally wherein transcription of one or more of CasA, B, C, D and E of a Type I CRISPR/Cas system is repressed.
7. The vector of any preceding Clause, wherein Cas modification of the host cell genome
   (a) kills the host cell;
   (b) reduces growth or proliferation of the cell or episome;
   (c) increases growth or proliferation of the cell or episome;
   (d) reduces or prevents transcription of a nucleotide sequence that comprises or is adjacent a said protospacer sequence; or
   (e) increases transcription of a nucleotide sequence that comprises or is adjacent a said protospacer sequence.
8. The vector of any preceding Clause, wherein the repressor is H-NS, StpA, LRP or CRP.
9. The vector of any preceding Clause, wherein the de-repressor is a mutant H-NS, StpA, LRP or CRP that is capable of forming a complex with H-NS, StpA, LRP or CRP repressor respectively in the host cell to prevent or reduce repression of the CRISPR/Cas system.
10. The vector of any preceding Clause, wherein the de-repressor is LeuO or LysR or a functional equivalent thereof.
11. The vector of any preceding Clause, wherein the cell is a bacterial or archaeal cell.
12. The vector of any preceding Clause, wherein the vector comprises an expressible htpG sequence.
13. The vector of any preceding Clause, wherein the cell comprises a CRISPR/Cas system comprising Cascade and Cas3, wherein the Cascade is repressed in the host cell, wherein the vector comprises
   (i) An expressible nucleotide sequence encoding a de-repressor of said Cascade repression; and
   (ii) An expressible nucleotide sequence encoding a Cas3, wherein the Cas 3 is capable of functioning with de-repressed Cascade in the host cell; Wherein the nucleotide sequences are capable of being expressed in the host cell.
14. The vector of Clause 13, wherein the nucleotide sequences of (i) and (ii) are under the control of one or more constitutive promoters that is (are) operable in the cell.
15. A nucleic acid vector (optionally according to any preceding Clause) for introduction into a bacterial or archaeal host cell, wherein the cell comprises an endogenous CRISPR/Cas system that is naturally repressed by a repressor in the cell, the vector comprising
   (a) A nucleotide sequence encoding a de-repressor that is capable of de-repressing the CRISPR/Cas system in the cell, wherein the sequence is expressible in the cell to produce the de-repressor; and
   (b) A CRISPR array for production of one or more crRNAs in the cell; and/or one or more nucleotide sequences encoding a respective guide RNA (gRNA) in the cell; wherein each cRNA or gRNA is capable of guiding Cas to modify a protospacer sequence of the host cell genome or to modify a protospacer sequence of an episome comprised by the host in the presence of the de-repressor;
   Wherein
   (c) the repressor is H-NS, StpA, LRP or CRP or a functional equivalent thereof encoded by the cell genome;
   (d) nucleotide sequence (a) comprises a constitutive promoter or strong promoter for expression of the sequence in the host cell; and
   (e) nucleotide sequence or array (b) comprises a constitutive promoter or strong promoter for expression of the sequence in the host cell.
16. The vector of Clause 15, wherein the de-repressor is a LeuO or a functional equivalent thereof; or a mutant of the repressor; or a siRNA that is complementary to a nucleotide sequence comprised by the host cell encoding the repressor.
17. The vector of any preceding Clause, wherein the cell is an E coli, Streptococcus or Salmonella cell, optionally an EHEC E coli or S enteric serovar typhimurium cell.
18. The vector of any preceding Clause, wherein the protospacer sequence is a chromosomal sequence, an endogenous host cell sequence, a wild-type host cell sequence, a non-viral chromosomal host cell sequence, not an exogenous sequence and/or a non-phage sequence.
19. The vector of any preceding Clause, wherein the CRIPSR/Cas system comprises a Cas3 and a repressed Cascade and the de-repressor is capable of de-repressing the Cascade in the cell, wherein the nucleotide sequence or array (b) comprises a CRISPR repeat sequence that is operable with the de-repressed CRISPR/Cas system, the repeat sequence comprising or consisting of a sequence selected from SEQ ID NOs: 49-52, or a sequence that is at least 70, 75, 80, 85, 90, 95, 96, 97, 98 or 99% identical to said selected sequence.
20. The vector of Clause 19, wherein the Cas3 comprises an amino acid sequence selected from SEQ ID NO: 58 or 60, or a sequence that is at least 70, 75, 80, 85, 90, 95, 96, 97, 98 or 99% identical to said selected sequence.
21. The vector of Clause 19 or 20, wherein the Cas3 is operable with a PAM comprising or consisting of the nucleotide sequence AWG.
22. The vector of Clause 19, 20 or 21, wherein the Cascade comprises a repressed CasA and the de-repressor is capable of de-repressing the CasA, the CasA comprising an amino acid sequence selected from SEQ ID NO: 66 or 68, or a sequence that is at least 70, 75, 80, 85, 90, 95, 96, 97, 98 or 99% identical to said selected sequence.
23. The vector of any one of Clauses 1 to 18, wherein the cell is a S enterica cell and the CRISPR/Cas system comprises a type E (Cse) Cas that is repressed, wherein the de-repressor is capable of de-repressing the Cas, optionally wherein the de-repressor is a LeuO or a functional equivalent thereof.
24. The vector of any one of Clauses 1 to 18 and 23, wherein the CRIPSR/Cas system comprises a Cas3 and a repressed Cascade and the de-repressor is capable of de-repressing the Cascade in the cell, wherein the nucleotide sequence or array (b) comprises a CRISPR repeat sequence that is operable with the de-repressed CRISPR/Cas system, the repeat sequence comprising or consisting of SEQ ID NO: 53, or a sequence that is at least 70, 75, 80, 85, 90, 95, 96, 97, 98 or 99% identical thereto.
25. The vector of Clause 24, wherein the Cas3 comprises an amino acid sequence selected from SEQ ID NO: 56 or 64, or a sequence that is at least 70, 75, 80, 85, 90, 95, 96, 97, 98 or 99% identical to said selected sequence.
26. The vector of Clause 24 or 25, wherein the Cascade comprises a repressed CasA and the de-repressor is capable of de-repressing the CasA, the CasA comprising an amino acid sequence selected from SEQ ID NO: 70 or 72, or a sequence that is at least 70, 75, 80, 85, 90, 95, 96, 97, 98 or 99% identical to said selected sequence.
27. The vector of any preceding Clause, wherein the nucleotide sequence or array (b) comprises a CRISPR repeat sequence that is operable with the de-repressed CRISPR/Cas system in the cell, the repeat sequence being at least 90% identical to a repeat in a host array comprised by the CRISPR/Cas system of the cell, wherein the vector or sequence or array (b) does not comprise a PAM recognised by a Cas nuclease of the host CRISPR/Cas system.
28. The vector of any preceding Clause, wherein the vector comprises no sequences from the group consisting of CasA, B, C, D and E nucleotide sequences, or wherein the vector does not comprise all of the sequences of said group.
29. The vector of any one of Clauses 1 to 28, wherein the vector comprises no sequences from the group consisting of Cas1, Cas2, Cas5 and Cas6 sequences.
30. The vector of any preceding Clause, wherein the vector comprises no Cas 3 nucleotide sequence.
31. The vector of any preceding Clause for medical use for treating or preventing a disease or condition in a human or animal subject, wherein the host cell is comprised by the subject.
32. The vector of any preceding Clause for medical use for killing said host cell or for reducing the growth or proliferation thereof in a human or animal microbiome.
33. The vector of Clause 32, wherein the microbiome comprises a plurality of said host cells and comprises further cells of a species or strain that is different from the species or strain of the host cells, wherein the further cells do not comprise the protospacer sequence.
34. A plurality of bacteriophage or phagemids comprising a plurality of vectors of any preceding Clause, optionally wherein the vectors are identical.
35. The plurality of bacteriophage or phagemids of Clause 34 when dependent from Clause
33, wherein the phage are capable of infecting the host cells but are not capable of infecting the further cells, or the phagemids are comprised by such phage.
36. A medicament comprising a plurality of vectors, bacrteriophage or phagemids according to any preceding Clause, optionally further comprising one or more medical drugs or antibiotics, for treating or preventing a disease or condition in a human or animal.
37. A method of treating or preventing a disease or condition in a human or animal subject, the method comprising administering a vector, plurality of vectors or medicament of any preceding Clause to the subject, wherein host cells comprised by a microbiome of the subject are modified by endogenous de-repressed Cas of the cells, and the treatment or prevention is carried out.
38. The method of Clause 37, wherein the method kills wild-type E coli or Salmonella host cells.
39. A nucleic acid vector for introduction into a host cell, wherein the cell comprises a CRISPR/Cas system that is repressed by a repressor in the cell, the vector comprising
   (a) A nucleotide sequence encoding a de-repressor that is capable of de-repressing the CRISPR/Cas system in the cell, wherein the sequence is expressible in the cell to produce the de-repressor; and
   (b) A site for introduction of
      (i) a CRISPR array or a CRISPR spacer sequence for production of one or more crRNAs in the cell;
      (ii) a nucleotide sequence encoding a guide RNA (gRNA) in the cell;
         wherein said cRNA or gRNA is capable of guiding Cas to modify a respective protospacer sequence of the host cell genome or to modify a protospacer sequence of an episome comprised by the host in the presence of the de-repressor.
40. The vector of Clause 39, wherein insertion of (i) or (ii) into said site forms a vector according to any one of Clauses 1 to 33.
41. A medicament comprising a plurality of nucleic acid vectors for introduction into bacterial or archaeal host cells, optionally further comprising one or more medical drugs or antibiotics, for treating or preventing a disease or condition in a human or animal; wherein
   (i) each cell comprises a CRISPR/Cas system that is repressed by a repressor selected from H-NS and/or StpA in the cell,
   (ii) the vector comprises a nucleotide sequence encoding a de-repressor that is capable of de-repressing the CRISPR/Cas system in the cell, wherein the sequence is expressible in the cell to produce the de-repressor optionally under the control of a strong and/or constitutive promoter; and
   the vector being devoid of a CRISPR array for production of one or more crRNAs in the cell; and devoid of one or more nucleotide sequences encoding a respective guide RNA (gRNA) in the cell.
42. The vector, bacteriophage, phagemids, medicament or method of any preceding Clause, wherein a Cascade Cas, Cas3 or Cas9 is repressed.
43. The vector, bacteriophage, phagemids, medicament or method of any preceding Clause, wherein the system comprises a
   (a) Cas that comprises the amino acid sequence selected from SEQ ID NO: 58, 60, 66 and 68, or an amino acid sequence that is at least 70, 75, 80, 85, 90, 95, 96, 97, 98 or 99% (eg, at least 80%) identical to the selected sequence, or is an orthologue or homologue thereof that is operable with a repeat comprising a sequence selected from SEQ ID NOs: 49-52 and a PAM comprising or consisting of AWG; or
   (b) Cas that comprises the amino acid sequence selected from SEQ ID NO: 56, 64, 70 and 72, or an amino acid sequence that is at least 70, 75, 80, 85, 90, 95, 96, 97, 98 or 99% (eg, at least 80%) identical to the selected sequence, or is an orthologue or homologue thereof that is operable with a repeat comprising SEQ ID NO: 53; or
   (c) Cas that comprises the amino acid sequence selected from SEQ ID NO:62, or an amino acid sequence that is at least 70, 75, 80, 85, 90, 95, 96, 97, 98 or 99% (eg, at least 80%) identical to the selected sequence, or is an orthologue or homologue thereof that is operable with a PAM comprising or consisting of NNAGAAW, NGGNG or AW.
44. The vector, bacteriophage, phagemids, medicament or method of any preceding Clause, wherein the system comprises
   (a) a repeat comprising a sequence selected from SEQ ID NOs: 49-52 (or a sequence that is at least 70, 75, 80, 85, 90, 95, 96, 97, 98 or 99% identical to said selected sequence) and a PAM comprising or consisting of AWG;
   (b) a repeat comprising SEQ ID NO: 53 (or a sequence that is at least 70, 75, 80, 85, 90, 95, 96, 97, 98 or 99% identical to said selected sequence); or
   (c) a PAM comprising or consisting of NNAGAAW, NGGNG or AW.
45. The vector, bacteriophage, phagemids, medicament or method of any preceding Clause, wherein the system comprises a repressed Cas and the Cas is operable with
   (a) a PAM comprising or consisting of AWG, and optionally the Cas nuclease comprises the amino acid sequence selected from SEQ ID NO: 58, 60, 66 and 68, or an amino acid sequence that is at least 70, 75, 80, 85, 90, 95, 96, 97, 98 or 99% (eg, at least 80%) identical to the selected sequence; or
   (b) a PAM comprising or consisting of NNAGAAW, NGGNG or AW and optionally the Cas nuclease comprises the amino acid sequence selected from SEQ ID NO: 62, or an amino acid sequence that is at least 70, 75, 80, 85, 90, 95, 96, 97, 98 or 99% (eg, at least 80%) identical to the selected sequence.
46. The vector, bacteriophage, phagemids, medicament or method of any preceding Clause, wherein the repressor comprises
   (i) an amino acid sequence selected from SEQ ID NO: 17, 19, 21, 23, 25 and 27, or an amino acid sequence that is at least 70, 75, 80, 85, 90, 95, 96, 97, 98 or 99% (eg, at least 80%) identical to said selected sequence; or
   (ii) an amino acid sequence selected from SEQ ID NO: 29, 31, 33 and 35, or an amino acid sequence that is at least 70, 75, 80, 85, 90, 95, 96, 97, 98 or 99% (eg, at least 80%) identical to said selected sequence.
47. The vector, bacteriophage, phagemids, medicament or method of any preceding Clause, wherein the de-repressor comprises
   (i) an amino acid sequence selected from SEQ ID NO: 3, 5, 7, 9, 11, 13 and 15, or an amino acid sequence that is at least 70, 75, 80, 85, 90, 95, 96, 97, 98 or 99% (eg, at least 80%) identical to said selected sequence; or
   (ii) an amino acid sequence selected from SEQ ID NO: 37, 39 and 41, or an amino acid sequence that is at least 70, 75, 80, 85, 90, 95, 96, 97, 98 or 99% (eg, at least 80%) identical to said selected sequence; or
   (iii) an amino acid sequence selected from SEQ ID NO: 43, 45 and 47, or an amino acid sequence that is at least 70, 75, 80, 85, 90, 95, 96, 97, 98 or 99% (eg, at least 80%) identical to said selected sequence.
48. The vector, bacteriophage, phagemids, medicament or method of any preceding Clause, wherein the or each vector is devoid of a Cas-encoding nucleotide sequence or a nucleotide sequence encoding a repressed Cas of the system.

Providing the de-repressor-encoding sequence and the crRNA or gRNA-encoding sequence on the same vector is useful for ensuring that these means for bringing about Cas-mediated modification of the host cell genome are introduced into the cell simultaneously. Thus, this ensures that all cells in which de-repression is effected will also be supplied with the means for Cas-mediated modification. If separate vectors were used instead for the various components, then this would be more difficult to ensure and control; some cells may receive the de-repressor, but not the crRNA/gRNA or vice versa. The invention, by ensuring that the components are delivered together, is useful for addressing wild-type host cells where pre-modification to genomically encode the crRNA/gRNA-encoding sequence is not possible; such cells may, for example, be comprised by a microbiome of a human, animal or natural environment (eg, soil or a waterway or water source). The invention configurations where the de-repressor-encoding sequence and the crRNA or gRNA-encoding sequence are comprised by the same vector are therefore useful methods of medicine practised on humans or animals. These configurations, further, are useful because the potential for co-transfer of the sequences into the target host cells enables more predictable dosing of each sequence, and thus allows for more reliable dosing for medical or other uses of compositions to recipient host cells, eg, comprised by microbiomes. Furthermore, configurations where the de-repressor-encoding sequence and the crRNA or gRNA-encoding sequence are comprised by the same vector allow for co-control of the expression of the sequences, eg, by a common promoter or by designing the vector so that these sequences are comprised by the same operon. For example, an inducible promoter could be used wherein vectors of the invention have been introduced into host cells, where provision of the inducing agent (eg, administration to a human or animal to which vectors have or are administered) switches on the promoter for simultaneous expression of the de-repressor and crRNA/gRNA.

In other embodiments, the use of a constitutive promoter is advantageous, as this ensures expression of the de-repressor and crRNA/gRNA in the host cells, which then increases the chances that the de-repressed Cas will be guided by the crRNA or gRNA of the invention (to cut or otherwise modify the target protospacer), rather than guided by endogenously-produced crRNA or gRNA. For medical use, eg, where the vectors are administered to a human or animal subject (such as to a gut microbiome thereof), it may be useful to ensure constitutive expression to control dosing and to maximise the chances that vectors reaching their target in the body will be effective, rather than trying to rely on switching on activity by administering an inducer (where instead an inducible promoter is to be used) in the hope that the inducer reaches the target cells and in an effective dose for effective induction and production of effective levels of de-repressor and crRNA/gRNA in the microbiome. Similarly, a strong promoter is useful to increase the chances of de-reperession and also to increase the chances that desirable expression of crRNA/gRNA of the invention is high (and possibly also out-produces that background level of endogenously-encoded crRNA or gRNA). Thus, the use of the strong promoter increases that chances that cutting (or other modification) of the target protospacer will happen in host cells.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications and all US equivalent patent applications and patents are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” or similar as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non limiting Examples.

### EXAMPLES

### Example 1: Medical Use

An application of the invention provides a plurality of vectors, medicaments and methods as described herein for treating, preventing or reducing (eg, reducing spread of or expansion of) a host cell bacterial infection in a human or animal.

In a first example, a host cell modifying (HM)-array and LeuO-encoding nucleotide sequence of the invention is contained in a population of Class I, II or III Staphylococcus packaged phage (Caudovirales or Myoviridae phage). The phage population is administered to a MRSA-infected patient with or without methicillin or vancomycin. In one trial, the phage HM-arrays target (i) the region of 20 nucleotides at the 3′ of the leader promoter of endogenous S aureus CRISPR arrays and (ii) the methicillin resistance genes in the host cells. When vancomycin is administered, a lower dose than usual is administered to the patient. It is expected that host cell infection will be knocked-down and resistance to the phage medicine will not be established or established at a lower rate or severity than usual. In other trials, the design is identical except that the phage in those trials also target the essential S aureus gene ftsZ (Liang et al, Int J Infect Dis. 2015 January; 30:1-6. doi: 10.1016/j.ijid.2014.09.015. Epub 2014 Nov. 5, “Inhibiting the growth of methicillin-resistant Staphylococcus aureus in vitro with antisense peptide nucleic acid conjugates targeting the ftsZ gene”). LeuO is expressed in the host Staphylococcus cells and de-represses H-NS repressed Cas in the host cells. The phage vectors are devoid of any Cas-encoding sequence, but instead crRNAs expressed from the HM-array operate with endogenous Cas encoded by the host genome.

A further trial will repeat the trials above, but phage K endolysin was administered in addition or instead of methicillin.

### Example 2: Selective Bacterial Population Growth Inhibition in a Mixed Consortium of Different Microbiota Species

We demonstrated selective growth inhibition of a specific bacterial species in a mixed population of three species. We selected species found in gut microbiota of humans and animals (S thermophilus DSM 20617(T), Lactobacillus lactis and E coli). We included two gram-positive species (the S thermophilus and L lactis) to see if this would affect the ability for selective killing of the former species; furthermore to increase difficulty (and to more closely simulate situations in microbiota) L lactis was chosen as this is a phylogenetically-related species to S thermophilus (as indicated by high 16s ribosomal RNA sequence identity between the two species). The S thermophilus and L lactis are both Firmicutes. Furthermore, to simulate microbiota, a human commensal gut species (E coli) was included.

1. Materials & Methods

Methods as set out in Example 6 of US20160333348 were used strain (except that selective media was TH media supplemented with 2.5 g 1⁻¹ of 2-phenylethanol (PEA)).

1.1 Preparation of Electro-Competent L. lactis Cells

Overnight cultures of L. lactis in TH media supplemented with 0.5 M sucrose and 1% glycine were diluted 100-fold in 5 ml of the same media and grown at 30° C. to an OD₆₀₀ between 0.2-0.7 (approximately 2 hours after inoculation). The cells were collected at 7000×g for 5 min at 4° C. and washed three times with 5 ml of ice cold wash buffer (0.5 M sucrose+10% glycerol). After the cells were washed, they were suspended to an OD₆₀₀ of 15-30 in electroporation buffer (0.5 M sucrose, 10% glycerol and 1 mM MgCl₂). The cells in the electroporation buffer were kept at 4° C. until use (within one hour) or aliquot 50 μl in eppendorf tubes, freezing them in liquid nitrogen and stored at −80° C. for later use.

Electroporation conditions for all species were as described in Example 6 of US20160333348.

1.2 Activation of CRISPR Array: Consortium Experiments.

S thermophilus DSM 20617, L. lactis MG1363 and E. coli TOP10 were genetically transformed with the plasmid containing the CRISPR array targeting the DNA polymerase III and tetA of S thermophilus. After transformation all cells were grown alone and in co-culture for 3 hours at 37° C. allowing for recovery to develop the antibiotic resistance encoded in the plasmid. We decided to use transformation efficiency as a read out of CRISPR-encoded growth inhibition. Therefore, after allowing the cells for recovery the cultures were plated in TH media, TH supplemented with PEA and MacConkey agar all supplemented with Kanamycin, and induced by 1% xylose.

2. Results

2.0 Phylogenetic Distance Between L. lactis, E. Coli and S thermophilus

The calculated sequence similarity in the 16S rrNA-encoding DNA sequence of the S thermophilus and L. lactis was determined as 83.3%. The following 16S sequences were used: E. coli: AB030918.1, S thermophilus: AY188354.1, L. lactis: AB030918. The sequences were aligned with needle (http://www.ebi.ac.uk/Tools/psa/emboss_needle/nucleotide.html) with the following parameters: -gapopen 10.0-gapextend 0.5-endopen 10.0-endextend 0.5-aformat3 pair-snucleotidel-snucleotide2. FIG. 11 of US20160333348 shows the maximum-likelihood phylogenetic tree of 16S sequences from S thermophilus, L. lactis and E. coli.

2.1 Growth Condition and Selective Media

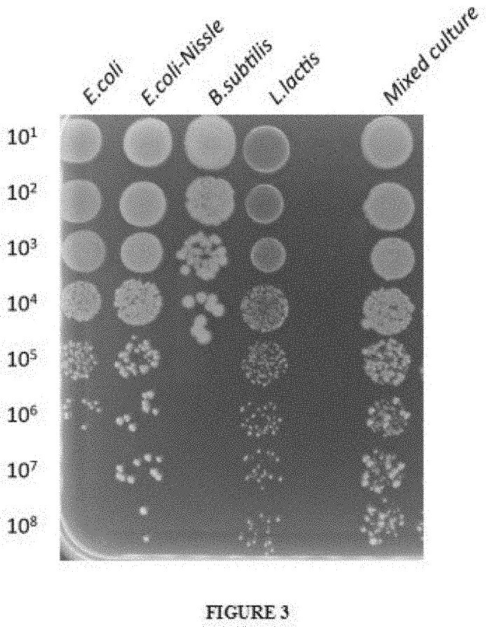

S thermophilus and L. lactis are commonly used in combination in many fermented foods and yoghurt. We chose these strains since they are commonly known to be gut microbes that form an intimate association with the host and previous characterizations of the 16S ribosomal RNA region of S thermophilus and L. lactis have shown that these organisms are phylogenetically closely related (Ludwig et al., 1995). In parallel we also evaluated the growth of E. coli for our mixed population co-culture experiments, since this organism is also commonly found in gut microbe communities. We first set out to establish the bacterial strains and cultivation protocol that would support growth for all strains we planned to use for the co-cultivation experiments. We found that all strains were able to support growth in TH broth at 37° C. (FIG. 3 of US20160333348).

Distinguishing the different bacteria from a mixed culture is important in order to determine cell number of the different species. With MacConkey agar is possible to selectively grow E. coli, however there is no specific media for selective growth of S. thermophilus. PEA agar is a selective medium that is used for the isolation of gram-positive (S thermophilus) from gram-negative (E. coli). Additionally, different concentrations of PEA partially inhibit the growth of the different grams positive species and strains, which allow for selection between the other gram-positive bacteria used in this work. Using 2.5 g 1⁻¹ of PEA proved to selectively grow S thermophilus while limiting growth of L. lactis and E. coli.

All strains were transformed with a plasmid that used the vector backbone of pBAV1KT5 that has a kanamycin selection marker; we found that using media supplemented with 30 μg ml⁻¹ of kanamycin was enough to grow the cells while keeping the plasmid.

2. 3 Transformation & Selective Growth Inhibition in a Mixed Population

We transformed S thermophilus, L. lactis and E. coli with plasmid containing the CRISPR array and cultured them in a consortium of all the bacterial species combined in equal parts, which would allow us to determine if we could cause cell death specifically in S. thermophilus. We transformed all the species with either the pBAV1KT5-XylR-CRISPR-P_{XylA} or pBAV1KT5-XylR-CRISPR-P_{ldha+Xy/A} plasmid.

FIG. 12 of US20160333348 shows the selective S thermophilus growth inhibition in a co-culture of E. coli, L. lactis and S thermophilus harboring either the pBAV1KT5-XylR-CRISPR-P_{xylA} or the pBAV1KT5-XylR-CRISPR-P_{ldhA+XylA} plasmid. No growth difference is observed between E. coli harboring the pBAV1KT5-XylR-CRISPR-P_{xylA}, or the pBAV1KT5-XylR-CRISPR-P_{ldhA+XylA} plasmid (middle column) However, S. thermophilus (selectively grown on TH agar supplemented with 2.5 gl⁻¹ PEA, last column) shows a decrease in transformation efficiency between the pBAV1KT5-XylR-CRISPR-P_{xylA} (strong) or the pBAV1KT5-XylR-CRISPR-P_{ldhA+XylA} (weak) plasmid as we expected. We thus demonstrated a selective growth inhibition of the target S thermophilus sub-population in the mixed population of cells.

Targeting E coli in Mixed Consortia by Harnessing De-Repressed Endogenous Cas

An illustrative application of this example of the invention is the targeting of E coli cells comprised by a mixed bacterial population comprising at least 3 different bacterial species, by the introduction of one or more vectors of the invention into an E coli cell (eg, an Escherichia coli O157 H7 EDL933 (EHEC) cell) that comprises a repressed Cas3 and/or Cascade (or a CasA thereof) (wherein H-NS represses the Cas and/or Cascade). The vector(s) comprise (a) a nucleotide sequence encoding a de-repressor (such as LeuO) that is capable of de-repressing the Cascade or Cas in the cell, wherein the sequence is expressible in the cell to produce the de-repressor; and (b) a CRISPR array for production of one or more crRNAs in the cell; and/or one or more nucleotide sequences encoding a respective guide RNA (gRNA, eg, a single guide RNA) in the cell; wherein each crRNA or gRNA is capable of guiding the Cas or a Cas of the Cascade to modify a respective protospacer sequence of the host cell genome or to modify a protospacer sequence of an episome comprised by the host in the presence of the de-repressor. Component (b) comprises a CRISPR repeat sequence that is operable with the de-repressed Cas or Cascade, eg, the repeat sequence comprises or consists of a sequence selected from SEQ ID NO: 49-52. In an example, the de-repressed Cas is a Cas3 comprising an amino acid sequence of SEQ ID NO: 58 or 60. In an example, the de-repressed Cas is a CasA comprising an amino acid sequence of SEQ ID NO: 66 or 68. Optionally, the target nucleotide sequence or protospacer comprises the sequence of at least 5, 6, 7, 8, 9 or 10 contiguous nucleotides immediately 3′ of a PAM in the genome of the host cell, wherein the PAM is selected from AWG, AAG, AGG, GAG and ATG.

Targeting S enterica in Mixed Consortia by Harnessing De-Repressed Endogenous Cas

An illustrative application of this example of the invention is the targeting of S enterica cells comprised by a mixed bacterial population comprising at least 3 different bacterial species, by the introduction of one or more vectors of the invention into an S enterica cell (eg, a Salmonella enterica subsp. enterica serovar Typhimurium cell, eg, Salmonella enterica subsp. enterica serovar Typhimurium LT2 cell or Salmonella enterica subsp. enterica serovar Typhimurium Paratyphi A cell) that comprises a repressed Cas3 and/or Cascade (or a CasA thereof) (wherein H-NS represses the Cas and/or Cascade). The vector(s) comprise (a) a nucleotide sequence encoding a de-repressor (such as LeuO) that is capable of de-repressing the Cascade or Cas in the cell, wherein the sequence is expressible in the cell to produce the de-repressor; and (b) a CRISPR array for production of one or more crRNAs in the cell; and/or one or more nucleotide sequences encoding a respective guide RNA (gRNA, eg, a single guide RNA) in the cell; wherein each crRNA or gRNA is capable of guiding the Cas or a Cas of the Cascade to modify a respective protospacer sequence of the host cell genome or to modify a protospacer sequence of an episome comprised by the host in the presence of the de-repressor. Component (b) comprises a CRISPR repeat sequence that is operable with the de-repressed Cas or Cascade, eg, the repeat sequence comprises or consists of SEQ ID NO: 53. In an example, the de-repressed Cas is a Cas3 comprising an amino acid sequence of SEQ ID NO: 56 or 64. In an example, the de-repressed Cas is a CasA comprising an amino acid sequence of SEQ ID NO: 70 or 72. Optionally, the target nucleotide sequence or protospacer comprises the sequence of at least 5, 6, 7, 8, 9 or 10 contiguous nucleotides immediately 3′ of a PAM in the genome of the host cell, wherein the PAM is operable with the Cas3.

### Example 3: Vector-Encoded System for Selective Species & Strain Growth Inhibition in a Mixed Bacterial Consortium

In Example 2 we surprisingly established the possibility of harnessing endogenous Cas nuclease activity in host bacteria for selective population growth inhibition in a mixed consortium of different species. We next explored the possibility of instead using vector-encoded Cas activity for selective population growth inhibition in a mixed consortium of different species. We demonstrated selective growth inhibition of a specific bacterial species in a mixed population of three different species, and further including a strain alternative to the target bacteria. We could surprisingly show selective growth inhibition of just the target strain of the predetermined target species. Furthermore, the alternative strain was not targeted by the vector-encoded CRISPR/Cas system, which was desirable for establishing the fine specificity of such vector-borne systems in a mixed bacterial consortium that mimicked human or animal gut microbiota elements.

We selected species found in gut microbiota of humans and animals (Bacillus subtilis, Lactobacillus lactis and E coli). We included two strains of the human commensal gut species, E coli. We thought it of interest to see if we could distinguish between closely related strains that nevertheless had sequence differences that we could use to target killing in one strain, but not the other. This was of interest as some strains of E coli in microbiota are desirable, whereas others may be undesirable (eg, pathogenic to humans or animals) and thus could be targets for Cas modification to knock-down that strain.

### 1. Material and Methods

### 1.1. Plasmids and Strains

All strains were cultivated in Todd-Hewitt broth (TH) (T1438 Sigma-Aldrich), in aerobic conditions and at 37° C., unless elsewhere indicated. The strains were stored in 25% glycerol at −80° C.

The self-targeting sgRNA-Cas9 complex was tightly regulated by a theophylline riboswitch and the AraC/P_{BAD} expression system respectively. Tight regulation of Cas9 is desired in order to be carried stably in E. coli. The plasmid contained the exogenous Cas9 from Streptococcus pyogenes with a single guide RNA (sgRNA) targeting E. coli's K-12 strains. Therefore K-12 derived strains TOP10 was susceptible to double strand self-cleavage and consequent death when the system was activated. E. coli strains like Nissle don't have the same target sequence therefore they were unaffected by the sgRNA-Cas9 activity. See Tables 9-11 in U.S. Ser. No. 15/478,912 (filed 4^{th} April 2017, and incorporated herein by reference), which show sequences used. We chose a target sequence (ribosomal RNA-encoding sequence) that is conserved in the target cells and present in multiple copies (7 copies), which increased the chances of cutting host cell genomes in multiple places to promote killing using a single gRNA design.

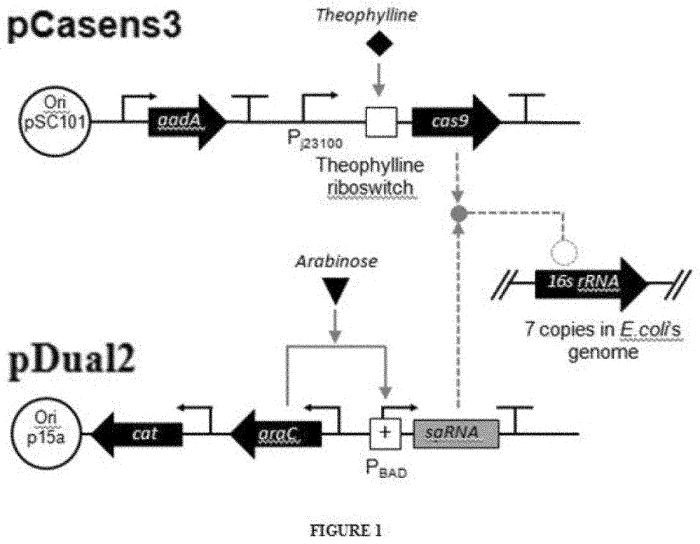

FIG. 1 shows regulators controlling the expression of spCas9 and the self-targeting sgRNA targeting the ribosomal RNA subunit 16s.

### 1. 2. Differential Growth Media

All strains were grown on TH media at 37° C. for 20 hours. Selective media for B. subtilis was TH media supplemented with 2.5 g 1⁻¹ of 2-phenylethanol (PEA). PEA was added to the media and autoclaved at 121° C. for 15 minutes at 15 psi. Agar plates were prepared by adding 1.5% (wt/vol) agar to the corresponding media.

### 1. 3. Cloning

E. coli (One Shot® ThermoFisher TOP10 Chemically Competent cells) was used in all subcloning procedures. PCR was carried out using Phusion™ polymerase. All PCR products were purified with Nucleospin™ Gel and PCR Clean-up by Macherey-Nagel™ following the manufacturer's protocol. The purified fragments were digested with restriction enzyme DpnI in 1×FD buffer with 1 μl enzyme in a total volume of 34 μl. The digested reaction was again purified with Nucleospin Gel and PCR Clean-up by Macherey-Nagel following the manufacturer's protocol. Gibson assembly was performed in 10 μl reactions following the manufacturer's protocol (New England Biolab).

Plasmid DNA was prepared using Qiagen kits according to the manufacturer's instructions. Modifications for Gram-positive strains included growing bacteria in a medium supplemented with 0.5% glycine and lysozyme to facilitate cell lysis.

### 1. 4. Transformation

1. 4.1 Electro-Competent E. coli Cells and Transformation

Commercially electrocompetent cells were used for cloning and the experiments (One Shot® Thermo Fisher TOP10 electrocompetent E. coli). Electroporation was done using standard settings: 1800 V, 25 μF and 200Ω using an Electro Cell Manipulator (BTX Harvard Apparatus ECM630). Following the pulse, 1 ml LB-SOC media was added and the cells were incubated at 37° C. for 1 hour. The transformed cells were plated in LB-agar containing the corresponding antibiotics.

1.5. Activation of sgRNA-Cas9 in E. coli and Consortium Experiments.

E. coli TOP10 and Nissle both with the plasmid containing the sgRNA targeting the ribosomal RNA-encoding sequence of K-12 derived strains and the other bacteria were grown overnight in 3 ml of TH broth. The next day the cells were diluted to ˜OD 0.5 and next 10-fold serially diluted in TH media and using a 96-well replicator (Mettler Toledo Liquidator™ 96) 4 μL volume drops were spotted on TH agar, TH agar with inducers (1% arabinose and 2 mM theophylline), TH agar supplemented with 2.5 g 1⁻¹ PEA and MacConkey agar supplemented with 1% maltose. The plates were incubated for 20 h at 37° C. and the colony forming units (CFU) were calculated from triplicate measurements.

### 2. Results

2.1 Specific Targeting of E. coli Strains Using an Exogenous CRISPR-Cas9 System

We first tested if the system could differentiate between two E. coli strains by introducing the killing system in both E. coli TOP10 and Nissle.

2.1 Targeting of E. coli Using an Exogenous CRISPR-Cas9 System in a Mixed Culture

Serial dilutions of overnight cultures were done in duplicate for both E. coli strains, B. subtilis, L. lactis, and in triplicate for the mixed cultures. All strains were grown at 37° C. for 20 hours in selective plates with and without the inducers. Induction of the system activates the sgRNA-Cas9 targeting K-12 derived strains, while leaving intact the other bacteria.

Distinguishing the different bacteria from a mixed culture is important in order to determine cell numbers of the different species and determine the specific removal of a species. MacConkey agar selectively grows E. coli, PEA agar is a selective medium that is used for the isolation of gram-positive (B. subtilis) from gram-negative (E. coli). Additionally, we found that different concentrations of PEA partially inhibit the growth of other gram positives. 2.5 g 1⁻¹ of PEA proved to selectively grow B. subtilis while limiting growth of E. coli and L. lactis.

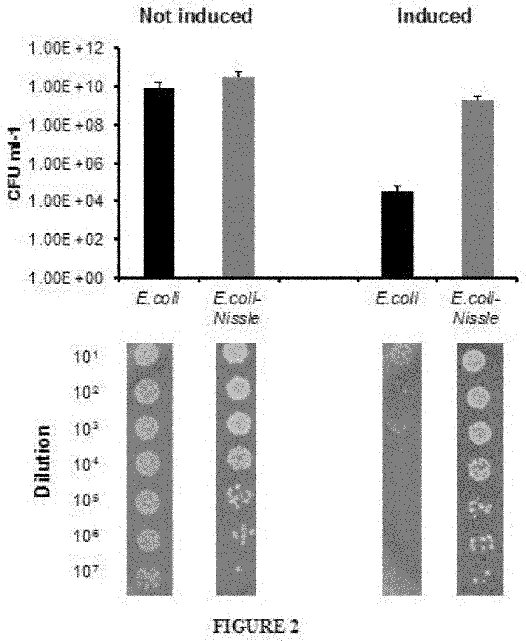

FIG. 2 shows specific targeting of E. coli strain by the inducible, exogenous, vector-borne CRISPR-Cas system. The sgRNA target the genome of K-12 derived E. coli strain E. coli TOP10, while the other E. coli strain tested was unaffected.

FIG. 3 shows spot assay with serial dilutions of individual bacterial species used in this study and mixed culture in TH agar without induction of the CRISPR-Cas9 system.

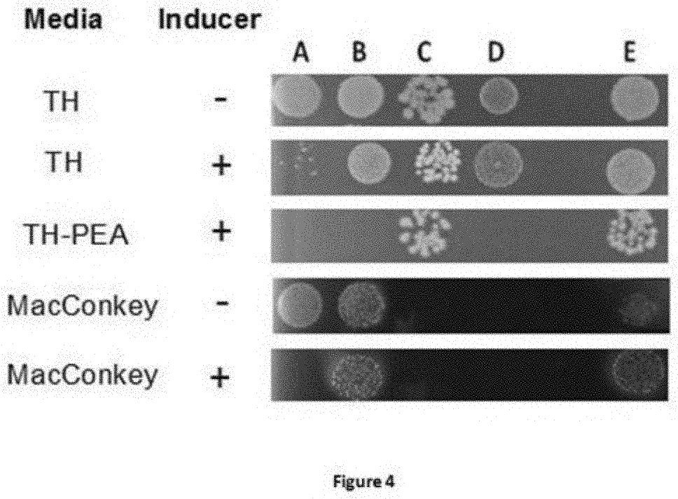

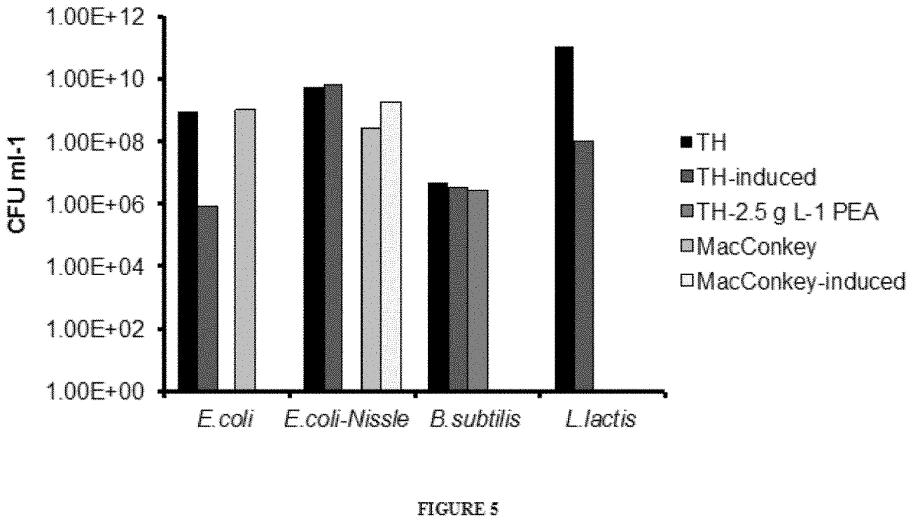

FIG. 4 shows a spot assay of the dilution 10³ on different selective media. TH with 2.5 g 1⁻¹ PEA is a selective media for B. subtilis alone. MacConkey supplemented with maltose is a selective and differential culture medium for bacteria designed to selectively isolate Gram-negative and enteric bacilli and differentiate them based on maltose fermentation. Therefore TOP10 ΔmalK mutant makes white colonies on the plates while Nissle makes pink colonies; A is E coli ΔmalK, B is E coli Nissile, C is B subtilis, D is L lactis, E is mixed culture; the images at MacConkey−/B and E appear pink; the images at MacConkey+/B and E appear pink. FIG. 5 shows selective growth of the bacteria used in this study on different media and selective plates. It can be seen that we clearly, selectively killed the target E coli strain (“E coli” on x-axis in FIG. 5) in the mixed population, whereas the other related strain (“E coli-Nissle”) was not similarly killed Killing of the target strain in the mixed population was 1000-fold in this experiment.

Targeting E coli in Mixed Consortia by Harnessing De-Repressed Exogenous Cas

An illustrative application of this example of the invention is the targeting of E coli cells comprised by a mixed bacterial population comprising at least 3 different bacterial species, by the introduction of one or more vectors of the invention into an E coli cell (eg, an Escherichia coli O157 H7 EDL933 (EHEC) cell) that comprises a repressed Cas9, such as a spCas9 or stCas9 (wherein H-NS represses the Cas). The Cas9 is encoded by a nucleotide sequence that was comprised by a vector introduced into the host cell (eg, on the same or different vector as a repressor-encoding sequence). Vector(s) are introduced into the host cell that comprise (a) a nucleotide sequence encoding a de-repressor (such as LeuO) that is capable of de-repressing Cas9 in the cell, wherein the sequence is expressible in the cell to produce the de-repressor; and (b) a CRISPR array for production of one or more crRNAs in the cell; and/or one or more nucleotide sequences encoding a respective guide RNA (gRNA, eg, a single guide RNA) in the cell; wherein each crRNA or gRNA is capable of guiding the Cas9 to modify a respective protospacer sequence of the host cell genome or to modify a protospacer sequence of an episome comprised by the host in the presence of the de-repressor. Component (b) comprises a CRISPR repeat sequence that is operable with the de-repressed Cas9, eg, the Cas9 is S pyogenes Cas9. Optionally, the target nucleotide sequence or protospacer comprises the sequence of at least 5, 6, 7, 8, 9 or 10 contiguous nucleotides immediately 5′ of a PAM in the genome of the host cell, wherein the PAM is NGG.

### REFERENCES

[1] Zhang, X. Z., & Zhang, Y. H. P. (2011). Simple, fast and high-efficiency transformation system for directed evolution of cellulase in Bacillus subtilis. Microbial Biotechnology, 4(1), 98-105. http://doi.org/10.1111/j.1751-7915.2010.00230.x
[2] Wegmann, U., O'Connell-Motherway, M., Zomer, A., Buist, G., Shearman, C., Canchaya, C., . . . Kok, J. (2007). Complete genome sequence of the prototype lactic acid bacterium Lactococcus lactis subsp. cremoris MG1363. Journal of Bacteriology, 189(8), 3256-70. http://doi.org/10.1128/JB.01768-06.

### SEQUENCE LISTING

TABLE 1
Example Repressors and De-Repressors

| SEQ ID NO: | DESCRIPTION | SEQUENCE |
|---|---|---|
| 1 | UP-IGLB (Used as | TTG TTC TCC TTC ATA TGC TCC GAC ATT TCT |
| | upstream primer for | |
| | cloning of the IGLB | |
| | region) | |
| 2 | DOWN-IGLB (Used | CTT CGG GAA TGA TTG TTA TCA ATG ACG ATA |
| | as downstream primer | |
| | for cloning of the | |
| | IGLB region) | |
| 3 | Escherichia coli K-12 | MPEVQTDHPETAELSKPQLRMVDLNLLTVFDAVMQEQNITRAAHVLGMSQPAVSNAVARL |
| | MG1655 LeuO | KVMFNDELFVRYGRGIQPTARAFQLFGSVRQALQLVQNELPGSGFEPASSERVFHLCVCS |
| | | PLDSILTSQIYNHIEQIAPNIHVMFKSSLNQNTEHQLRYQETEFVISYEDFHRPEFTSVP |
| | | LFKDEMVLVASKNHPTIKGPLLKHDVYNEQHAAVSLDRFASFSQPWYDTVDKQASIAYQG |
| | | MAMMSVLSVVSQTHLVAIAPRWLAEEFAESLELQVLPLPLKQNSRTCYLSWHEAAGRDKG |
| | | HQWMEEQLVSICKR |
| 4 | Escherichia coli K-12 | atgccagaggtacaaacagatcatccagagacggcggagttaagcaaaccacagctacgc |
| | MG1655 LeO | atggtcgatctcaacttattaaccgttttcgatgccgtgatgcaggagcaaaacattact |
| | | cgtgccgctcatgttctgggaatgtcgcaacctgcggtcagtaacgctgttgcacgcctg |
| | | aaggtgatgtttaatgacgagctttttgttcgttatggccgtggtattcaaccgactgct |
| | | cgcgcatttcaactttttggttcagttcgtcaggcattgcaactagtacaaaatgaattg |
| | | cctggttcaggttttgaacccgcgagcagtgaacgtgtatttcatctttgtgtttgcagc |
| | | ccgttagacagcattctgacctcgcagatttataatcacattgagcagattgcgccaaat |
| | | atacatgttatgttcaagtcttcattaaatcagaacactgaacatcagctgcgttatcag |
| | | gaaacggagtttgtgattagttatgaagacttccatcgtcctgaatttaccagcgtacca |
| | | ttatttaaagatgaaatggtgctggtagccagcaaaaatcatccaacaattaagggcccg |
| | | ttactgaaacatgatgtttataacgaacaacatgcggcggtttcgctcgatcgtttcgcg |
| | | tcatttagtcaaccttggtatgacacggtagataagcaagccagtatcgcgtatcagggc |
| | | atggcaatgatgagcgtacttagcgtggtgtcgcaaacgcatttggtcgctattgcgccg |
| | | cgttggctggctgaagagttcgctgaatccttagaattacaggtattaccgctgccgtta |
| | | aaacaaaacagcagaacctgttatctctcctggcatgaagctgccgggcgcgataaaggc |
| | | catcagtggatggaagagcaattagtctcaatttgcaaacgctaa |
| 5 | Escherichia coli O157 | MTVELSMPEVQTDHPETAEFSKPQLRMVDLNLLTVFDAVMQEQNITRAAHVLGMSQPAVS |
| | H7 EDL933 (EHEC) | NAVARLKVMFNDELFVRYGRGIQPTARAFQLFGSVRQALQLVQNELPGSGFEPASSERVF |
| | LeuO | HLCVCSPLDSILTSQIYNHIEQIAPNIHVMFKSSLNQNTEHQLRYQETEFVISYEDFHRP |
| | | EFTSVPLFKDEMVLVASKNHPTIKGPLLKHDVYNEQHAAVSLDRFASFSQPWYDTVDKQA |
| | | SIAYQGMAMMSVLSVVSQTHLVAIAPRWLAEEFAESLELQVLPLPLKLNSRTCYLSWHEA |
| | | AGRDKGHQWMEEQLVSICKR |
| 6 | Escherichia coli O157 | gtgacagtggagttaagtatgccagaggtacaaacagatcatccagagacggcggagttc |
| | H7 EDL933 (EHEC) | agcaagccacagctacgcatggtcgatctcaacttattaaccgttttcgatgccgtgatg |
| | LeuO | caggagcaaaacattacccgtgctgctcatgttctgggaatgtcgcaacctgcggtcagt |
| | | aacgctgttgcacgcctgaaggtgatgtttaatgacgagctttttgttcgttatggccgt |
| | | ggtattcaaccgactgctcgcgcatttcaactttttggttcagttcgtcaggcattgcaa |
| | | ctagtacaaaatgaattgcctggttcaggttttgaacccgcgagcagtgaacgtgtattt |
| | | catctttgtgtttgcagcccgttagacagtattctgacctcgcagatttataatcacatt |
| | | gagcagattgcgccaaatatacatgttatgttcaagtcttcattaaatcagaacactgaa |
| | | catcagctgcgttatcaggaaacggagtttgtgattagttatgaagacttccatcgtcct |
| | | gaatttaccagcgtgccattatttaaagatgaaatggtgctggtagccagcaaaaatcac |
| | | ccaacaattaagggcccgttactgaaacatgatgtttataacgaacaacatgcggcggtt |
| | | tcgctcgatcgtttcgcgtcatttagtcaaccttggtatgacacggtagataagcaagcc |
| | | agtatcgcgtatcagggcatggcaatgatgagcgtacttagcgtggtgtcgcaaacgcat |
| | | ttggtcgctattgcgccgcgttggctggctgaagagttcgctgaatccttagaattacag |
| | | gtattaccgctgccgttaaaactaaatagcagaacctgttatctctcctggcatgaagct |
| | | gccgggcgtgataaaggccatcagtggatggaagagcaattagtctcaatttgcaaacgc |
| | | taa |
| 7 | Salmonella enterica | MPEVKTEKPHLLDMGKPQLRMVDLNLLTVFDAVMQEQNITRAAHTLGMSQPAVSNAVARL |
| | subsp. enterica | KVMFNDELFVRYGRGIQPTARAFQLFGSVRQALQLVQNELPGSGFEPTSSERVFNLCVCS |
| | serovar Typhi CT18: | PLDNILTSQIYNRVEKIAPNIHVVFKASLNQNTEHQLRYQETEFVISYEEFRRPEFTSVP |
| | STY0134 LeuO | LFKDEMVLVASRKHPRISGPLLEGDVYNEQHAVVSLDRYASFSRPWYDTPDKQSSVAYQG |
| | | MALISVLNVVSQTHLVAIAPCWLAEEFAESLELQILPLPLKLNSRTCYLSWHEAAGRDKG |
| | | HQWMEDLLVSVCKR |
| 8 | Salmonella enterica | atgccagaggtcaaaaccgaaaagccgcatcttttagatatgggcaaaccacagcttcgc |
| | subsp. enterica | atggttgatttgaacctattgaccgtgttcgatgcggtaatgcaagagcagaatattacg |
| | serovar Typhi CT18: | cgcgccgcccacacgctgggaatgtcgcagcctgcggtcagtaacgccgtagcgcgtctg |
| | STY0134 LeuO | aaggttatgtttaatgacgaactttttgttcgatatggacgaggaattcagccgactgcc |
| | | cgtgcatttcagttatttggttcagtccgtcaggcgttgcaattggtgcaaaatgaattg |
| | | ccgggatcggggtttgagccgaccagcagcgaacgtgtattcaatctttgcgtgtgcagt |
| | | ccgctggataatatcctgacgtcacagatttataatcgtgtagaaaaaattgcgccaaat |
| | | attcatgtcgtttttaaagcgtcgttgaatcagaatactgagcatcagttacgctatcag |
| | | gaaaccgagttcgttattagttatgaagaattccgtcgtcctgagtttaccagcgtaccg |
| | | ctatttaaagatgaaatggttttagtcgccagccgaaaacacccgcgtattagcggcccg |
| | | ctactggaaggcgatgtttataatgaacaacatgcggttgtttctctcgatcgttatgcg |
| | | tcatttagtcggccgtggtatgacacgccggataaacagtcgagcgtggcttatcagggc |
| | | atggcgcttatcagcgttctgaacgtggtttcgcagacgcatttggtcgctattgccccg |
| | | tgctggctggcggaagagtttgcggagtcgctggagctgcaaatactgccgttgccttta |
| | | aaactgaatagccggacatgctacctttcctggcatgaagcggctgggcgtgataaaggg |
| | | catcaatggatggaagatttattagtctctgtttgtaagcgataa |
| 9 | Salmonella enterica | MPEVKTEKPHLLDMGKPQLRMVDLNLLTVFDAVMQEQNITRAAHTLGMSQPAVSNAVARL |
| | subsp. enterica | KVMFNDELFVRYGRGIQPTARAFQLFGSVRQALQLVQNELPGSGFEPTSSERVFNLCVCS |
| | serovar Typhimurium | PLDNILTSQIYNRVEKIAPNIHVVFKASLNQNTEHQLRYQETEFVISYEEFRRPEFTSVP |
| | LT2: STM0115 LeuO | LFKDEMVLVASRKHPRISGPLLEGDVYNEQHAVVSLDRYASFSQPWYDTPDKQSSVAYQG |
| | | MALISVLNVVSQTHLVAIAPRWLAEEFAESLDLQILPLPLKLNSRTCYLSWHEAAGRDKG |
| | | HQWMEDLLVSVCKR |
| 10 | Salmonella enterica | atgccagaggtcaaaaccgaaaagccgcatcttttagatatgggcaaaccacagcttcgc |
| | subsp. enterica | atggttgatttgaacctattgaccgtgttcgatgcggtaatgcaagagcagaatattacg |
| | serovar Typhimurium | cgcgccgcccacacgctgggaatgtcgcagcctgcggtcagtaacgccgtagcgcgtctg |
| | LT2: STM0115 LeuO | aaggttatgtttaatgacgaactttttgttcgatatggacgaggaattcagccgactgcc |
| | | cgtgcatttcagttatttggttcagtccgtcaggcgttgcaattggtgcaaaatgaattg |
| | | ccgggatcggggtttgagccgaccagcagcgaacgtgtattcaatctttgcgtgtgcagt |
| | | ccgctggataatatcctgacgtcacagatttataatcgtgtagaaaaaattgcgccaaat |
| | | attcatgtcgtttttaaagcgtcgttgaatcagaatactgagcatcagttacgctatcag |
| | | gaaaccgagttcgttattagttatgaagaattccgtcgtcctgagtttaccagcgtaccg |
| | | ctatttaaagatgaaatggttttagtcgccagccgaaaacacccgcgtattagcggcccg |
| | | ctactggaaggcgatgtttataatgaacaacatgcggttgtttccctcgatcgttatgcg |
| | | tcatttagtcagccgtggtatgacacgccggataaacagtcgagcgtggcttatcagggc |
| | | atggcgcttatcagcgttctgaacgtggtttcgcagacgcatttggtcgctattgccccg |
| | | cgctggctggcggaagagtttgcggaatcgctggatctgcaaatattgccgttgccttta |
| | | aaactgaatagccggacatgctacctttcctggcatgaagcggctgggcgtgataaaggg |
| | | catcaatggatggaagatttattagtctctgtttgtaagcgataa |
| 11 | Salmonella enterica | MPEVKTEKPHLLDMGKPQLRMVDLNLLTVFDAVMQEQNITRAAHTLGMSQPAVSNAVARL |
| | subsp. enterica | KVMFNDELFVRYGRGIQPTARAFQLFGSVRQALQLVQNELPGSGFEPTSSERVFNLCVCS |
| | serovar Paratyphi A | PLDNILTSQIYNRVEKIAPNIHVVFKASLNQNTEHQLRYQETEFVISYEEFRRPEFTSVP |
| | ATCC9150: SPA0117 | LFKDEMVLVASRKHPRISGPLLEGDVYNEQHAVVSLDRYASFSQPWYDTPDKQSSVAYQG |
| | LeuO | MALISVLNVVSQTHLVAIAPRWLAEEFAESLELQILPLPLKLNSRTCYLSWHEAAGRDKG |
| | | HQWMEDLLVSVCKR |
| 12 | Salmonella enterica | atgccagaggtcaaaaccgaaaagccgcatcttttagatatgggcaaaccacagcttcgc |
| | subsp. enterica | atggttgatttgaacctattgaccgtgttcgatgcggtaatgcaagagcagaatattacg |
| | serovar Paratyphi A | cgcgccgcccacacgctgggaatgtcgcagcctgcggtcagtaacgccgtagcgcgtctg |
| | ATCC9150: SPA0117 | aaggttatgtttaatgacgaactttttgttcgatatggacgaggaattcagccgactgcc |
| | LeuO | cgtgcatttcagttatttggttcagtccgtcaggcgttgcaattggtgcaaaatgaattg |
| | | ccgggatcagggtttgagccgaccagcagcgaacgtgtattcaatctttgcgtgtgcagt |
| | | ccgctggataatatcctgacgtcacagatttataatcgtgtagaaaaaattgcgccaaat |
| | | attcatgtcgtttttaaagcgtcgttgaatcagaatactgagcatcagttacgctatcag |
| | | gaaaccgagttcgttattagttatgaagaattccgtcgtcctgagtttaccagcgtaccg |
| | | ctatttaaagatgaaatggttttagtcgccagccgaaaacacccgcgtattagcggcccg |
| | | ctactggaaggcgatgtttataatgaacaacatgcggttgtttctctcgatcgttatgcg |
| | | tcatttagtcagccgtggtatgacacgccggataaacagtcgagcgtggcttatcagggc |
| | | atggcgcttatcagcgttctgaacgtggtttcgcagacgcatttggtcgctattgccccg |
| | | cgctggctggcggaagagtttgcggagtcgctggagctgcaaatactgccgttgccttta |
| | | aaactgaatagccggacatgctacctttcctggcatgaagcggctgggcgtgataaaggg |
| | | catcaatggatggaagatttattagtttctgtttgtaagcgataa |
| 13 | Salmonella enterica | MPEVKTEKPHLLDMGKPQLRMVDLNLLTVFDAVMQEQNITRAAHTLGMSQPAVSNAVARL |
| | subsp. enterica | KVMFNDELFVRYGRGIQPTARAFQLFGSVRQALQLVQNELPGSGFEPTSSERVFNLCVCS |
| | serovar Enteritidis | PLDNILTSQIYNRVEKIAPNIHVVFKASLNQNTEHQLRYQETEFVISYEEFRRPEFTSVP |
| | OLF-SE1-1019-1: | LFKDEMVLVASRKHPRISGPLLEGDVYNEQHAVVSLDRYASFSQPWYDTPDKQSSVAYQG |
| | IY59_00600 LeuO | MALISVLNVVSQTHLVAIAPRWLAEEFAESLDLQILPLPLKLNSRTCYLSWHEAAGRDKG |
| | | HQWMEDLLVSVCKR |
| 14 | Salmonella enterica | atgccagaggtcaaaaccgaaaagccgcatcttttagatatgggcaaaccacagcttcgc |
| | subsp. enterica | atggttgatttgaacctattgaccgtgttcgatgcggtaatgcaagagcagaatattacg |
| | serovar Enteritidis | cgcgccgcccacacgctgggaatgtcgcagcctgcggtcagtaacgccgtagcgcgtctg |
| | OLF-SE1-1019-1: | aaggttatgtttaatgacgaactttttgttcgatatggacgaggaattcagccgactgcc |
| | IY59_00600 LeuO | cgtgcatttcagttatttggttcagtccgtcaggcgttacaattggtgcaaaatgaattg |
| | | ccgggatcggggtttgagccgaccagcagcgaacgtgtattcaatctttgcgtgtgcagt |
| | | ccgctggataatatcctgacgtcacagatttataatcgtgtagaaaaaattgcgccaaat |
| | | attcatgtcgtttttaaagcgtcgttgaatcagaatactgagcatcagttacgctatcag |
| | | gaaaccgagttcgttattagttatgaagaattccgtcgtcctgagtttaccagcgtaccg |
| | | ctatttaaagatgaaatggttttagtcgccagccgaaaacacccgcgtattagcggcccg |
| | | ctactggaaggcgatgtttataatgaacaacatgcggttgtttctctcgatcgttatgcg |
| | | tcatttagtcagccgtggtatgacacgccggataaacagtcgagcgtggcttatcagggc |
| | | atggcgcttatcagcgttctgaacgtggtttcgcagacgcatttggtcgctattgccccg |
| | | cgctggctggcggaagagtttgcggaatcgctggatctgcaaatattgccgttgccttta |
| | | aaactgaatagccggacatgctacctttcctggcatgaagcggctgggcgtgataaaggg |
| | | caccaatggatggaagatttattagtttctgtttgtaagcgataa |
| 15 | Shigella flexneri 301 | MTHSTAMDSVFIRTRIFMFSEFYSFCFFLFYMHDKSYSSGLFLCIPIRERELSVTVELSM |
| | (serotype 2a): SF0071 | PEVQTDHSETAELSKPQLRMVDLNLLTVFDAVMQEQNITRAAHVLGMSQPAVSNAVARLK |
| | LeuO | VMFNDELFVRYGRGIQPTARAFQLFGSVRQALQLVQNELPGSGFEPASSERVFHLCVCSP |
| | | LDSILTSQIYNHIEQIAPNIHVMFKSSLNQNTEHQLRYQETEFVISYEDFHRPEFTSVPL |
| | | FKDEMVLVASKNHPTIKGPLLKHDVYNEQHAAVSLDRFASFSQPWYDTVDKQASIAYQGM |
| | | AMMSVLSVVSQTHLVAIAPRWLAEEFAESLELQVLPLPLKQNSRTCYLSWHEAAGRDKGH |
| | | QWMEEQLVSICKR |
| 16 | Shigella flexneri 301 | atgactcattccacggcaatggattctgtttttatcagaacccgtatctttatgttttcc |
| | (serotype 2a): SF0071 | gaattttactcattttgctttttcttattttatatgcatgataaatcatattcttcagga |
| | LeuO | ttatttctctgcattccaataagggaaagggagttaagtgtgacagtggagttaagtatg |
| | | ccagaggtacaaacagatcattcagagacggcggagttaagcaagccacagctacgcatg |
| | | gtcgatctcaacttattaaccgttttcgatgccgtgatgcaggagcaaaacattacccgt |
| | | gccgctcatgttctgggtatgtcgcaacctgcggtcagtaacgctgttgcacgcctgaag |
| | | gtgatgtttaatgacgagctttttgttcgttatggccgtggtattcaaccgactgctcgc |
| | | gcatttcaactttttggttcagttcgccaggcattgcaactagtacaaaatgaattgcct |
| | | ggttcaggttttgaacccgcgagcagtgaacgtgtatttcatctttgtgtttgcagcccg |
| | | ttagacagcattctgacctcgcagatttataatcacattgagcagattgcgccaaatata |
| | | catgttatgttcaagtcttcattaaatcagaacactgaacatcagctgcgttatcaggaa |
| | | acggagtttgtgattagttatgaagacttccatcgtcctgaatttaccagcgtgccatta |
| | | tttaaagatgaaatggtgctggtagccagcaaaaatcatccaacaattaaaggcccgtta |
| | | ctgaaacatgatgtttataacgaacaacatgcggcggtttcgctcgatcgtttcgcgtca |
| | | tttagtcaaccttggtatgacacggtagataagcaagccagtatcgcgtatcagggcatg |
| | | gcaatgatgagcgtacttagcgtggtgtcgcaaacgcatttggtcgctattgcgccgcgt |
| | | tggctggctgaagagttcgctgaatccttagaattacaggtattaccgctgccgttaaaa |
| | | caaaacagcagaacctgttatctctcttggcatgaagctgccgggcgcgataaaggccat |
| | | cagtggatggaagaacaattagtctcaatttgcaaacgctaa |
| 17 | Escherichia coli O157 | MSEALKILNNIRTLRAQARECTLETLEEMLEKLEVVVNERREEESAAAAEVEERTRKLQQ |
| | H7 EDL933 (EHEC): | YREMLIADGIDPNELLNSLAAVKSGTKAKRAQRPAKYSYVDENGETKTWTGQGRTPAVIK |
| | Z2013 H-NS | KAMDEQGKSLDDFLIKQ |
| 18 | Escherichia coli O157 | atgagcgaagcacttaaaattctgaacaacatccgtactcttcgtgcgcaggcaagagaa |
| | H7 EDL933 (EHEC): | tgtacacttgaaacgctggaagaaatgctggaaaaattagaagttgttgttaacgaacgt |
| | Z2013 H-NS | cgcgaagaagaaagcgcggctgctgctgaagttgaagagcgcactcgtaaactgcagcaa |
| | | tatcgcgaaatgctgatcgctgacggtattgacccgaacgagctgctgaatagccttgcc |
| | | gccgttaaatctggcaccaaagctaaacgtgctcagcgtccggcaaaatatagctacgtt |
| | | gacgaaaacggcgaaactaaaacctggactggccagggccgtactccagctgtaatcaaa |
| | | aaagcaatggatgagcaaggtaaatccctcgacgatttcctgatcaagcaataa |
| 19 | Escherichia coli O127 | MSEALKILNNIRTLRAQARECTLETLEEMLEKLEVVVNERREEESAAAAEVEERTRKLQQ |
| | H6 E2348/69 (EPEC): | YREMLIADGIDPNELLNSLAAVKSGTKAKRAQRPAKYSYVDENGETKTWTGQGRTPAVIK |
| | E2348C_1364 H-NS | KAMDEQGKSLDDFLIKQ |
| 20 | Escherichia coli O127 | atgagcgaagcacttaaaattctgaacaacatccgtactcttcgtgcgcaggcaagagaa |
| | H6 E2348/69 (EPEC): | tgtacacttgaaacgctggaagaaatgctggaaaaattagaagttgttgttaacgaacgt |
| | E2348C_1364 H-NS | cgcgaagaagaaagcgcggctgctgctgaagttgaagagcgcactcgtaaactgcagcaa |
| | | tatcgcgaaatgctgatcgctgacggtattgacccgaacgaactgctgaatagccttgct |
| | | gccgttaaatctggcaccaaagctaagcgtgctcagcgtccggcaaaatatagctacgtt |
| | | gacgaaaacggcgaaactaaaacctggactggccagggccgtactccagctgtaatcaaa |
| | | aaagcaatggatgagcaaggtaaatccctcgacgatttcctgatcaagcaataa |
| 21 | Salmonella enterica | MSEALKILNNIRTLRAQARECTLETLEEMLEKLEVVVNERREEESAAAAEVEERTRKLQQ |
| | subsp. enterica | YREMLIADGIDPNELLNSMAAAKSGTKAKRAARPAKYSYVDENGETKTWTGQGRTPAVIK |
| | serovar Typhi CT18: | KAMEEQGKQLEDFLIKE |
| | STY1299 H-NS | |
| 22 | Salmonella enterica | atgagcgaagcacttaaaattctgaacaacatccgtactcttcgtgcgcaggcaagagaa |
| | subsp. enterica | tgtactctggaaacgcttgaagaaatgctggaaaaattagaagttgtcgttaatgagcgt |
| | serovar Typhi CT18: | cgtgaagaagaaagcgctgctgctgctgaagtggaagaacgcactcgtaaactgcaacag |
| | STY1299 H-NS | tatcgtgaaatgttaattgccgacggcattgacccgaatgaactgctgaatagcatggct |
| | | gccgctaaatccggtaccaaagctaaacgcgcagctcgtccggctaaatatagctatgtt |
| | | gacgaaaacggtgaaactaaaacctggactggccagggtcgtacaccggctgtaatcaaa |
| | | aaagcaatggaagaacaaggtaagcaactggaagatttcctgatcaaggaataa |
| 23 | Salmonella enterica | MSEALKILNNIRTLRAQARECTLETLEEMLEKLEVVVNERREEESAAAAEVEERTRKLQQ |
| | subsp. enterica | YREMLIADGIDPNELLNSMAAAKSGTKAKRAARPAKYSYVDENGETKTWTGQGRTPAVIK |
| | serovar Typhimurium | KAMEEQGKQLEDFLIKE |
| | LT2: STM1751 H-NS | |
| 24 | Salmonella enterica | atgagcgaagcacttaaaattctgaacaacatccgtactcttcgtgcgcaggcaagagaa |
| | subsp. enterica | tgtactctggaaacgcttgaagaaatgctggaaaaattagaagttgtcgttaatgagcgt |
| | serovar Typhimurium | cgtgaagaagaaagcgctgctgctgctgaagtggaagaacgcactcgtaaactgcaacag |
| | LT2: STM1751 H-NS | tatcgtgaaatgttaattgccgacggcattgacccgaatgaactgctgaatagcatggct |
| | | gccgctaaatccggtaccaaagctaaacgcgcagctcgtccggctaaatatagctatgtt |
| | | gacgaaaacggtgaaactaaaacctggactggccagggtcgtacaccggctgtaatcaaa |
| | | aaagcaatggaagaacaaggtaagcaactggaagatttcctgatcaaggaataa |
| 25 | Salmonella enterica | MSEALKILNNIRTLRAQARECTLETLEEMLEKLEVVVNERREEESAAAAEVEERTRKLQQ |
| | subsp. enterica | YREMLIADGIDPNELLNSMAAAKSGTKAKRAARPAKYSYVDENGETKTWTGQGRTPAVIK |
| | serovar Enteritidis | KAMEEQGKQLEDFLIKE |
| | EC20090193: | |
| | AU37_06605 H-NS | |
| 26 | Salmonella enterica | atgagcgaagcacttaaaattctgaacaacatccgtactcttcgtgcgcaggcaagagaa |
| | subsp. enterica | tgtactctggaaacgcttgaagaaatgctggaaaaattagaagttgtcgttaatgagcgt |
| | serovar Enteritidis | cgtgaagaagaaagcgctgctgctgctgaagtggaagaacgcactcgtaaactgcaacag |
| | EC20090193: | tatcgtgaaatgttaattgccgacggcattgacccgaatgaactgctgaatagcatggct |
| | AU37_06605 H-NS | gccgctaaatccggtaccaaagctaaacgcgcagctcgtccggctaaatatagctatgtt |
| | | gacgaaaacggtgaaactaaaacctggactggccagggtcgtacaccggctgtaatcaaa |
| | | aaagcaatggaagaacaaggtaagcaactggaagatttcctgatcaaggaataa |
| 27 | Shigella flexneri | MSEALKILNNIRTLRAQARECTLETLEEMLEKLEVVVNERREEESAAAAEVEERTRKLQQ |
| | 2457T (serotype 2a): | YREMLIADGIDPNELLNSLAAVKSGTKAKRAQRPAKYSYVDENGETKTWTGQGRTPAVIK |
| | S1323 H-NS | KAMDEQGKSLDDFLIKQ |
| 28 | Shigella flexneri | atgagcgaagcacttaaaattctgaacaacatccgtactcttcgtgcgcaggcaagagaa |
| | 2457T (serotype 2a): | tgtacacttgaaacgctggaagaaatgctggaaaaattagaagttgttgttaacgaacgt |
| | S1323 H-NS | cgcgaagaagaaagcgcggctgctgctgaagttgaagagcgcactcgtaagctgcagcaa |
| | | tatcgcgaaatgctgatcgctgacggtattgacccgaacgaactgctgaatagccttgct |
| | | gccgttaaatctggcaccaaagctaaacgtgctcagcgtccggcaaaatatagctacgtt |
| | | gacgaaaacggcgaaactaaaacctggactggccaaggccgtactccagctgtaatcaaa |
| | | aaagcaatggatgagcaaggtaaatccctcgacgatttcctgatcaagcaataa |
| 29 | Escherichia coli K-12 | MSVMLQSLNNIRTLRAMAREFSIDVLEEMLEKFRVVTKERREEEEQQQRELAERQEKIST |
| | MG1655: b2669 StpA | WLELMKADGINPEELLGNSSAAAPRAGKKRQPRPAKYKFTDVNGETKTWTGQGRTPKPIA |
| | | QALAEGKSLDDFLI |
| 30 | Escherichia coli K-12 | atgtccgtaatgttacaaagtttaaataacattcgcaccctccgtgcgatggctcgcgaa |
| | MG1655: b2669 StpA | ttctccattgacgttcttgaagaaatgctcgaaaaattcagggttgtcactaaagaaaga |
| | | cgtgaagaagaagaacagcagcagcgtgaactggcagagcgccaggaaaaaattagcacc |
| | | tggctggagctgatgaaagctgacggaattaacccggaagagttattgggtaatagctct |
| | | gctgctgcaccacgcgctggtaaaaaacgccagccgcgtccggcgaaatataaattcacc |
| | | gatgttaacggtgaaactaaaacctggaccggtcagggccgtacaccgaagccaattgct |
| | | caggcgctggcagaaggtaaatctctcgacgatttcctgatctaa |
| 31 | Salmonella enterica | MNLMLQNLNNIRTLRAMAREFSIDVLEEMLEKFRVVTKERREEEELQQRQLAEKQEKINA |
| | subsp. enterica | FLELMKADGINPEELFAMDSAMPRSAKKRQPRPAKYRFTDFNGEEKTWTGQGRTPKPIAQ |
| | serovar Typhimurium | ALAAGKSLDDFLI |
| | LT2: STM2799 StpA | |
| 32 | Salmonella enterica | atgaatttgatgttacagaacttaaataatatccgcacgctgcgcgctatggctcgcgaa |
| | subsp. enterica | ttctccattgacgttcttgaagaaatgctcgaaaaattcagggttgtcactaaagaaaga |
| | serovar Typhimurium | cgcgaagaagaagaattgcagcaacgccagcttgccgagaagcaggagaaaattaatgcc |
| | LT2: STM2799 StpA | tttctggagctgatgaaagcagacggtattaacccggaagagttatttgccatggattca |
| | | gcaatgccgcgttctgctaaaaagcgccagccgcgtccggcaaaatatcgttttactgat |
| | | ttcaatggcgaagaaaaaacctggaccggacaaggtcgtacgcctaaaccgattgcccag |
| | | gcgctggcggcggggaaatctctggatgatttcttaatctaa |
| 33 | Salmonella enterica | MNLMLQNLNNIRTLRAMAREFSIDVLEEMLEKFRVVTKERREEEELQQRQLAEKQEKINA |
| | subsp. enterica | FLELMKADGINPEELFAMDSAMPRSAKKRQPRPAKYRFTDFNGEEKTWTGQGRTPKPIAQ |
| | serovar Typhimurium | ALAAGKSLDDFLI |
| | UK-1: STMUK_2788 | |
| | StpA | |
| 34 | Salmonella enterica | atgaatttgatgttacagaacttaaataatatccgcacgctgcgcgctatggctcgcgaa |
| | subsp. enterica | ttctccattgacgttcttgaagaaatgctcgaaaaattcagggttgtcactaaagaaaga |
| | serovar Typhimurium | cgcgaagaagaagaattgcagcaacgccagcttgccgagaagcaggagaaaattaatgcc |
| | UK-1: STMUK_2788 | tttctggagctgatgaaagcagacggtattaacccggaagagttatttgccatggattca |
| | StpA | gcaatgccgcgttctgctaaaaagcgccagccgcgtccggcaaaatatcgttttactgat |
| | | ttcaatggcgaagaaaaaacctggaccggacaaggtcgtacgcctaaaccgattgcccag |
| | | gcgctggcggcggggaaatctctggatgatttcttaatctaa |
| 35 | Shigella flexneri | MSVMLQSLNNIRTLRAMAREFSIDVLEEMLEKFRVVTKERREEEEQQQRELAERQEKIST |
| | 2457T (serotype 2a): | WLELMKADGINPEELLGNSSAAAPRAGKKRQPRPAKYKFTDVNGETKTWTGQGRTPKPIA |
| | S2883 StpA | QALAEGKSLDDFLI |
| 36 | Shigella flexneri | atgtccgtaatgttacaaagtttaaataacattcgcaccctccgtgcgatggctcgcgaa |
| | 2457T (serotype 2a): | ttctccattgacgttcttgaagaaatgctcgaaaaattcagggttgtcactaaagaaaga |
| | S2883 StpA | cgtgaagaagaagaacagcagcagcgtgaactggctgagcgtcaggaaaaaattagcacc |
| | | tggctggagctgatgaaagctgacggaattaacccggaagagttattgggtaatagctct |
| | | gctgctgcaccacgtgctggtaaaaaacgccagccgcgtccggcgaaatataaattcact |
| | | gatgttaacggtgaaactaaaacctggaccggtcagggccgtacaccgaagccaattgct |
| | | caggcgctggcagaaggtaaatctctcgacgatttcctgatctaa |
| 37 | Escherichia coli K-12 | MVDSKKRPGKDLDRIDRNILNELQKDGRISNVELSKRVGLSPTPCLERVRRLERQGFIQG |
| | MG1655.b0889 LRP | YTALLNPHYLDASLLVFVEITLNRGAPDVFEQFNTAVQKLEEIQECHLVSGDFDYLLKTR |
| | | VPDMSAYRKLLGETLLRLPGVNDTRTYVVMEEVKQSNRLVIKTR |
| 38 | Escherichia coli K-12 | atggtagatagcaagaagcgccctggcaaagatctcgaccgtatcgatcgtaacattctt |
| | MG1655: b0889 LRP | aatgagttgcaaaaggatgggcgtatttctaacgtcgagctttctaaacgtgtgggactt |
| | | tccccaacgccgtgccttgagcgtgtgcgtcggctggaaagacaagggtttattcagggc |
| | | tatacggcgctgcttaacccccattatctggatgcatcacttctggtattcgttgagatt |
| | | actctgaatcgtggcgcaccggatgtgtttgaacaattcaataccgctgtacaaaaactt |
| | | gaagaaattcaggagtgtcatttagtatccggtgatttcgactacctgttgaaaacacgc |
| | | gtgccggatatgtcagcctaccgtaagttgctgggggaaaccctgctgcgtctgcctggc |
| | | gtcaatgacacacggacatacgttgttatggaagaagtcaagcagagtaatcgtctggtt |
| | | attaagacgcgctaa |
| 39 | Salmonella enterica | MVDSKKRPGKDLDRIDRNILNELQKDGRISNVELSKRVGLSPTPCLERVRRLERQGFIQG |
| | subsp. enterica | YTALLNPHYLDASLLVFVEITLNRGAPDVFEQFNAAVQKLEEIQECHLVSGDFDYLLKTR |
| | serovar Typhimurium | VPDMSAYRKLLGETLLRLPGVNDTRTYVVMEEVKQSNRLVIKTR |
| | DT104: | |
| | DT104_09341 LRP | |
| 40 | Salmonella enterica | atggtagatagcaagaagcgccctggcaaagatctcgaccgtatcgatcgtaacattctt |
| | subsp. enterica | aatgaactgcaaaaggatgggcgtatttccaacgtcgagctttctaaacgagtaggactt |
| | serovar Typhimurium | tcgccgacaccttgccttgagcgtgtgcgtcggctggagcgacaggggtttatccagggc |
| | DT104: | tatacggcgctgttgaacccgcattatctggatgcgtcacttctggtattcgttgagatt |
| | DT104_09341 LRP | accttaaatcgcggcgcgccggatgtgtttgaacagtttaatgccgccgtgcaaaagctt |
| | | gaagagattcaggagtgtcatttggtttccggcgatttcgactacctgttgaaaacccgt |
| | | gtaccggatatgtcagcgtatcgaaaactattgggagagacgttgctgcgcttgccaggt |
| | | gtgaacgacacccgaacttacgtagtgatggaagaggtaaaacagagtaatcgtctggtt |
| | | attaagacacgctaa |
| 41 | Shigella flexneri | MVDSKKRPGKDLDRIDRNILNELQKDGRISNVELSKRVGLSPTPCLERVRRLERQGFIQG |
| | 2457T (serotype 2a): | YTALLNPHYLDASLLVFVEITLNRGAPDVFEQFNTAVQKLEEIQECHLVSGDFDYLLKTR |
| | S0889 LRP | VPDMSAYRKLLGETLLRLPGVNDTRTYVVMEEVKQSNRLVIKTR |
| 42 | Shigella flexneri | atggtagatagcaagaagcgccctggcaaagatctcgaccgtatcgatcgtaacattctt |
| | 2457T (serotype 2a): | aatgagttgcaaaaggatgggcgtatttctaacgtcgagctttctaaacgtgtgggactt |
| | S0889 LRP | tccccaacgccgtgccttgagcgtgtgcgtcggctggaaagacaagggtttattcagggc |
| | | tatacggcgctgcttaacccccattatctggatgcatcacttctggtattcgttgagatt |
| | | actctgaatcgtggcgcaccggatgtgtttgaacaattcaataccgctgtacaaaaactt |
| | | gaagaaattcaggagtgtcatttagtatctggtgatttcgactacctgttgaaaacacgc |
| | | gtgccggatatgtcagcttaccgtaagttgctgggggaaaccctgctgcgtctgcctggc |
| | | gtcaatgacacacggacatacgttgttatggaagaagtcaagcagagtaatcgtctggtt |
| | | attaagacgcgctaa |
| 43 | Escherichia coli K-12 | MVLGKPQTDPTLEWFLSHCHIHKYPSKSKLIHQGEKAETLYYIVKGSVAVLIKDEEGKEM |
| | W3110: JW5702 CRP | ILSYLNQGDFIGELGLFEEGQERSAWVRAKTACEVAEISYKKFRQLIQVNPDILMRLSAQ |
| | | MARRLQVTSEKVGNLAFLDVTGRIAQTLLNLAKQPDAMTHPDGMQIKITRQEIGQIVGCS |
| | | RETVGRILKMLEDQNLISAHGKTIVVYGTR |
| 44 | Escherichia coli K-12 | atggtgcttggcaaaccgcaaacagacccgactctcgaatggttcttgtctcattgccac |
| | W3110: JW5702 CRP | attcataagtacccatccaagagcaagcttattcaccagggtgaaaaagcggaaacgctg |
| | | tactacatcgttaaaggctctgtggcagtgctgatcaaagacgaagagggtaaagaaatg |
| | | atcctctcctatctgaatcagggtgattttattggcgaactgggcctgtttgaagagggc |
| | | caggaacgtagcgcatgggtacgtgcgaaaaccgcctgtgaagtggctgaaatttcgtac |
| | | aaaaaatttcgccaattgattcaggtaaacccggacattctgatgcgtttgtctgcacag |
| | | atggcgcgtcgtctgcaagtcacttcagagaaagtgggcaacctggcgttcctcgacgtg |
| | | acgggccgcattgcacagactctgctgaatctggcaaaacaaccagacgctatgactcac |
| | | ccggacggtatgcaaatcaaaattacccgtcaggaaattggtcagattgtcggctgttct |
| | | cgtgaaaccgtgggacgcattctgaagatgctggaagatcagaacctgatctccgcacac |
| | | ggtaaaaccatcgtcgtttacggcactcgttaa |
| 45 | Salmonella enterica | MVLGKPQTDPTLEWFLSHCHIHKYPSKSTLIHQGEKAETLYYIVKGSVAVLIKDEEGKEM |
| | subsp. enterica | ILSYLNQGDFIGELGLFEEGQERSAWVRAKTACEVAEISYKKFRQLIQVNPDILMRLSSQ |
| | serovar Typhimurium | MARRLQVTSEKVGNLAFLDVTGRIAQTLLNLAKQPDAMTHPDGMQIKITRQEIGQIVGCS |
| | DT104: | RETVGRILKMLEDQNLISAHGKTIVVYGTR |
| | DT104_34511 CRP | |
| 46 | Salmonella enterica | atggtgcttggcaaaccgcaaacagacccgactcttgaatggttcttgtctcattgccac |
| | subsp. enterica | attcataagtacccgtcaaagagcacgctgattcaccagggtgaaaaagcagaaacgctg |
| | serovar Typhimurium | tactacatcgttaaaggctccgtggcagtgctgatcaaagatgaagaagggaaagaaatg |
| | DT104: | atcctttcttatctgaatcagggtgattttattggtgaactgggcctgtttgaagaaggc |
| | DT104_34511 CRP | caggaacgcagcgcctgggtacgtgcgaaaaccgcatgtgaggtcgctgaaatttcctac |
| | | aaaaaatttcgccaattaatccaggtcaacccggatattctgatgcgcctctcttcccag |
| | | atggctcgtcgcttacaagtcacctctgaaaaagtaggtaacctcgccttccttgacgtc |
| | | accgggcgtatcgctcagacgctgctgaatctggcgaaacagcccgatgccatgacgcac |
| | | ccggatgggatgcagatcaaaatcactcgtcaggaaatcggccagatcgtcggctgctcc |
| | | cgcgaaaccgttggtcgtattttgaaaatgctggaagatcaaaacctgatctccgcgcat |
| | | ggcaagaccatcgtcgtctacggcacccgttaa |
| 47 | Shigella flexneri | MVLGKPQTDPTLEWFLSHCHIHKYPSKSTLIHQGEKAETLYYIVKGSVAVLIKDEEGKEM |
| | 2002017 (serotype | ILSYLNQGDFIGELGLFEEGQERSAWVRAKTACEVAEISYKKFRQLIQVNPDILMRLSAQ |
| | Fxv): SFxv_3687 | MARRLQVTSEKVGNLAFLDVTGRIAQTLLNLAKQPDAMTHPDGMQIKITRQEIGQIVGCS |
| | CRP | RETVGRILKMLEDQNLISAHGKTIVVYGTR |
| 48 | Shigella flexneri | atggtgcttggcaaaccgcaaacagacccgactctcgaatggttcttgtctcattgccac |
| | 2002017 (serotype | attcataagtacccatccaagagcacgcttattcaccagggtgaaaaagcggaaacgctg |
| | Fxv): SFxv_3687 | tactacatcgttaaaggctctgtggcagtgctgatcaaagacgaagagggtaaagaaatg |
| | CRP | atcctctcctatctgaatcagggtgattttattggcgaactgggcctgtttgaagagggc |
| | | caggaacgtagcgcatgggtacgtgcgaaaaccgcctgtgaagtggctgaaatttcgtac |
| | | aaaaaatttcgccaattgattcaggtaaacccggacattctgatgcgtctgtctgcacag |
| | | atggcgcgtcgtctgcaagtcacttcagagaaagtgggcaacctggcgttcctcgacgtg |
| | | acgggccgcattgcacagactctgctgaacctggcaaaacaaccagatgctatgactcac |
| | | ccggacggtatgcaaatcaaaattacccgtcaggaaatcggtcagattgtcggctgttct |
| | | cgtgaaaccgtgggacgcattctgaagatgctggaagatcagaacctgatctccgcacac |
| | | ggtaaaaccatcgtcgtttacggcactcgttaa |

TABLE 2
E coli Repeat Sequences

| SEQ | | |
|---|---|---|
| ID | Example | |
| NO: | Strain | Repeat |
| 49 | K12 | CGGTTTATCCCCGCTGGCGCGGGGAACTC |
| 50 | | GGTTTATCCCCGCTGGCGCGGGGAACAC |
| 51 | | CGGTTTATCCCCGCTGGCGCGGGGAAC |
| 52 | O157:H7 | CGGTTTATCCCCGCTGGCGCGGGGAACAC |

TABLE 3
Salmonella enterica subsp. enterica serovar
Typhimurium Repeat Sequences

| SEQ | | |
|---|---|---|
| ID | Example | |
| NO: | Strain | Repeat |
| 53 | UK-1 | CGGTTTATCCCCGCTGGCGCGGGGAACAC |

TABLE 4
Miscellaneous Sequences

| SEQ | | |
|---|---|---|
| ID | | |
| NO: | DESCRIPTION | SEQUENCE |
| 54 | E coli K12 | CTAAAAGTATACATTTGTTCTTAAAGCATT |
| | CRISPR I leader | |
| | sequence | |
| 55 | E coli K12 | TCTAAACATAACCTATTATTAATTAATGATTT |
| | CRISPR II | |
| | leader sequence | |
| 56 | Salmonella | MSIYHYWGKSRRGETDGGDDYHLLCWHSLDVAAVGYWMVINNIYFIDHYLKKLGIQDKEQ |
| | enterica subsp. | AAQFFAWILCWHDIGKFAHSFQQLYRHEALNIFNEPTRHYEKIAHTTLGYMLWNSWLSEC |
| | enterica serovar | PELFPPSSLSVRKSKRVMALWMPVTTGHHGRPPEAIQELDHFRQQDKDAARDFLLRIKAL |
| | Typhimurium | FPLITLPEAWDEDEGIDQFQQLSWFISAAVVLADWTGSASRYFPRTAEKMPVDTYWQQAL |
| | 14028S Cas3 | AKAQTAITLFPSAANVSAFTGIETLFPFIQHPTPLQQKALELDINVDGAQLFILEDVTGA |
| | | GKTEAALILAHRLMAAGKAQGLYFGLPTMATANAMFERMANTWLALYQPDSRPSLILAHS |
| | | ARRLMDRFNQSIWSVTLSGTEEPDEAQPYSQGCAAWFADSNKKALLAEVGVGTLDQAMM |
| | | A |
| | | VMPFKHNNLRLLGLSNKILLADEIHACDAWMSRILEGLIERQASNGNATILLSATLSQQQ |
| | | RDKLVAAFSRGVRRSVQAPLLGHDDYPWLTQVTQTELISQRVDTRKEVERCVDIGWLHSE |
| | | EACLERIGEAVEKGNCIAWIRNSVDDAIRIYRQLQLSKVVVTENLLLFHSRFAFYDRQRI |
| | | ESQTLNLFGKQSGAQRAGKVIIATQVIEQSLDIDCDEMISDLAPVDLLIQRAGRLQRHIR |
| | | DRNGLVKKSGQDERETPVLRILAPEWDDAPRENWLSSAMRNSAYVYPDHGRMWLTQRIL |
| | | R |
| | | EQGTIRMPQSARLLIESVYGEDVNMPVGFAKTEQLQEGKFYCDRAFAGQMLLNFAPGYCA |
| | | EISDSLPEKMSTRLAEESVTLWLAKIVDSVVTPYASGEHAWEMSVLRVRQSWWNKHKDEF |
| | | EKLDGEPLRKWCAQQHQDKDFATVIVVTDFAACGYSANEGLIGMMGE |
| 57 | Salmonella | gtgtcgatatatcactattggggaaagtctcgacgaggagaaactgacggcggtgatgat |
| | enterica subsp. | taccatttgctttgctggcattctttagatgttgcggctgtgggttactggatggtgata |
| | enterica serovar | aataatatttattttattgaccactatctaaaaaaattaggcatccaggataaggagcag |
| | Typhimurium | gcggcgcaattttttgcctggattttatgttggcatgatattggaaagtttgctcattcc |
| | 14028S Cas3 | ttccagcaactataccgtcatgaggctttaaatatctttaatgagcctacacggcattat |
| | nucleotide | gaaaaaatcgcgcataccacgctgggatacatgttgtggaactcctggctaagtgaatgc |
| | sequence | cctgaattgtttcctccttcttcgctttcagttcgtaaaagtaagcgcgttatggcgctt |
| | | tggatgccagtcactacaggtcatcatggacgccctccagaggcaatccaggagctggac |
| | | cattttcgccagcaggataaagacgcggcaagagattttcttctgagaataaaagcgctc |
| | | tttcctttaattactttgcctgaagcctgggatgaagatgagggtatcgaccaatttcag |
| | | caactttcctggtttatttccgctgcggttgtactggctgactggactggttctgccagc |
| | | cgttattttccgcgtactgcggaaaaaatgcctgttgatacctactggcagcaagctctc |
| | | gctaaagcacaaactgccatcacgctatttccctcagcggcgaatgtgtctgcctttacg |
| | | ggcatagaaacgcttttcccttttattcagcatcccacaccgttacaacaaaaggcgctt |
| | | gagctggatatcaacgtggatggcgcccaactctttattcttgaagatgtcaccggggcc |
| | | ggaaaaacagaggcggcgctcatattagctcatcgactgatggcggcaggtaaagcgcag |
| | | ggactctattttggactgccgacaatggcgacagccaacgcgatgtttgaacgtatggcg |
| | | aacacctggctggcgctgtatcagccggactcccgtcccagcctgattctggcgcatagc |
| | | gcgcgtcgcttaatggatcgtttcaatcagtcaatatggtcggtcactctttctggtacg |
| | | gaagaacccgatgaagcgcagccttatagtcagggatgcgccgcctggtttgccgacagc |
| | | aataaaaaagcgttgttggcggaggttggcgtaggcacgttggatcaggcgatgatggcg |
| | | gtaatgccatttaaacataacaacctgcggttactgggtcttagcaacaagatcttactg |
| | | gctgatgagatccatgcctgtgatgcctggatgtcccgaatacttgaaggtttgatcgaa |
| | | cggcaggccagtaatggcaacgccactattctgttatctgcgacgctatcgcagcagcag |
| | | cgagataagctggtggcggcattttcccgtggggtgaggcgtagtgtgcaggcgccgttg |
| | | ctaggccatgacgattatccctggctgactcaggtcacacaaacagagctgatttctcag |
| | | cgggttgatacacgcaaagaggttgagcgttgcgtagatattggctggctacatagtgaa |
| | | gaggcgtgtcttgaacgtataggtgaagcagtggaaaaaggaaactgtatcgcctggata |
| | | cgtaactccgttgatgatgcgattcgtatctatcgccagcttcaactgagtaaggtcgtc |
| | | gtcacggaaaaccttttactcttccatagtcgctttgctttttacgatcgtcagcggatt |
| | | gagtcacagacgctgaatctctttggcaaacagagcggcgcgcaacgtgccggtaaggtc |
| | | attatcgccacgcaggtcatcgaacaaagtctggatattgactgcgatgagatgatctct |
| | | gatttagcgccggtggatttattaattcagcgggccggtcgactacagcgtcatattcgc |
| | | gatcgtaacggtctggtgaaaaagagtgggcaggatgagcgagagacgccagtgctgcgc |
| | | attcttgctccggagtgggatgacgcgccgcgagagaactggttatccagcgccatgcgt |
| | | aacagcgcctatgtctatcccgatcatgggcgcatgtggctgacacagcgcatattacgt |
| | | gagcaggggacgattcggatgccgcaatctgcccgattgttgattgagtcggtctacggc |
| | | gaggatgtcaacatgccggttggatttgcaaaaaccgagcaattgcaggaaggcaaattt |
| | | tattgcgaccgggcatttgccggccagatgctgcttaactttgcgccgggctactgtgct |
| | | gaaattagcgattctttaccggagaaaatgtcaacgcggctggcggaagagtctgtcacg |
| | | ctgtggctggcgaaaatcgtggatagcgtcgtaaccccttatgccagcggtgaacacgcc |
| | | tgggagatgagcgtgctgcgagtacgtcagagctggtggaataaacataaagacgagttt |
| | | gaaaaattagacggcgaacccttgcgtaagtggtgtgcgcaacagcatcaggataaggat |
| | | tttgccacggtgattgtggtgacggactttgccgcttgtggttattcggcgaatgaggga |
| | | ttgattggcatgatgggggaataa |
| 58 | Escherichia coli | MRKYPLSLLKDKNIVTFFDFWGKTRRGEKEGGDGYHLLCWHSLDVAAMGYLMVKRNCFGL |
| | Cas3 | ADYFRQLGISDKEQAAQFFAWLLCWHDIGKFARSFQQLYLAPELKIPEGSRKNYEKISHS |
| | >ece:Z4070 | TLGYWLWNYYLSECEELLPSSSLSSRKLTRVIEMWMSITTGHHGRPPDRIDELDNFLPED |
| | K07012 | KAAARDFLLEIKALFPLIEIPTFWDDDEGVELLKQLSWYISATVVLADWTGSSTRFFPRV |
| | CRISPR- | AHPMDIKDYWQKTLVQAQNALTVFPPKAETAPFTGINTLFPFIEHPTPLQQKVLDLDISQ |
| | associated | PGPQLFILEDVTGAGKTEAALILAHRLMAARKAQGLFFGLPTMATANAMYDRLVKTWLAF |
| | endonuclease/ | YSPESRPSLVLAHSARTLMDRFNESLWSGDLVGSEEPDEQTFSQGCAAWFANSNKKALLA |
| | helicase Cas3 | EIGVGTLDQAMMAVMPFKHNNLRLLGLSNKILLADEIHACDAYMSCILEGLIERQARGGN |
| | [EC:3.1.-.- | SVILLSATLSQQQRDKLVAAFARGTEGQQEAPFLEKDDYPWLTHVTKSDVNSHRVATRKD |
| | 3.6.4.-]\| | VERSVSVGWLHSEQESIARIESAVSQGKCIAWIRNSVDDAIKVHRQLLARGVIPASSLSL |
| | (GenBank) | FHSRFAFSDRQRIEMETLARFGKEDGSQRAGKVLICTQVLEQSVDCDLDEMISDLAPVDL |
| | ygcB; orf; | LIQRAGRLQRHIRDINGQLKRDGKDERSPPELLILAPVWDDAPGDEWFGSAMRNSAYVYP |
| | hypothetical | DHGRIWLTQRVLREQGAIQMPHAARLLIESVYGEDVVMPEGFARSEQEQVGKYYCDRAMA |
| | protein (A) | KKFVLNFKPGYAANINDYLPEKLSTRLAEESVSLWLATCIAGVVKPYATGAHAWEMSVVR |
| | Strain O157:H7 | VRRSWWKKHRDEFSLLEGEAFRQWCIEQRQDPEMANVILVTDDESCGYSAREGLIGKVD |
| | EDL933 | |
| | (EHEC) | |
| 59 | Escherichia coli | atgcgtaaatatcctttaagtttactgaaggataaaaatattgtgactttctttgatttc |
| | Cas3 nucleotide | tggggaaaaacccgacgtggcgagaaagagggtggcgacggctatcaccttctttgctgg |
| | sequence | cattcgctggatgtggccgcaatgggctatttaatggttaaaagaaattgcttcgggctg |
| | Strain O157:H7 | gctgattactttcgtcaattagggatttctgacaaggaacaggcggctcaatttttcgct |
| | EDL933 | tggttgctgtgctggcacgatattggaaaatttgcccgctcttttcagcaactttacctg |
| | (EHEC) | gcccctgaactcaagattccggaaggttccagaaagaattacgaaaagatctctcattca |
| | >ece24070 | acgctgggttactggctgtggaattattatttaagtgaatgtgaggagttgcttccttca |
| | K07012 | tcttcactctcttctcgtaaacttacacgtgtaatagagatgtggatgtccataactacc |
| | CRISPR- | gggcatcatggtcgaccacctgaccgtattgatgagctggataattttctgcctgaagac |
| | associated | aaagctgccgcgcgagattttctccttgaaatcaaggcactgtttccgctcatagagatt |
| | endonuclease/ | cccacattctgggatgatgacgagggcgttgaacttttaaaacaactttcctggtatatc |
| | helicase Cas3 | tctgcaacagtcgtactcgcagactggacgggttcgtcaacgcgattttttccacgcgtc |
| | [EC:3.1.-.- | gcacacccaatggatattaaagattactggcagaaaactttagttcaggctcaaaacgcc |
| | 3.6.4.-]\| | ttaaccgtctttcctccaaaagcagaaaccgcacctttcaccggaattaatacgctgttt |
| | (GenBank) | ccttttattgagcacccgacaccattacagcaaaaggtactggatctggatatcagccag |
| | ygcB; oil; | ccagggccacagttatttattctggaagacgtgactggcgcaggtaaaacagaagcggcg |
| | hypothetical | cttatcctggcgcacaggttgatggctgcgaggaaagcacagggtttgttttttggcctg |
| | protein (N) | ccaacaatggcaacggccaatgccatgtacgatcggctggtcaaaacctggcttgctttc |
| | | tattcgccagagtcccgccccagcttggtgctggcacacagtgcccgcacattaatggac |
| | | cgcttcaatgaatcactctggtccggtgatttagtcgggtcagaagaaccggatgaacaa |
| | | acattcagtcagggatgtgcggcctggtttgccaacagtaacaagaaggcgctactggct |
| | | gaaattggcgtcggcacgctggatcaggcgatgatggcagtgatgccgtttaaacataat |
| | | aatctgcggcttctggggttgagtaacaaaatcctgctggctgatgagatccatgcctgt |
| | | gatgcttacatgtcgtgcattcttgaagggctgatcgagcggcaggcgcgtggcggaaac |
| | | agcgtcattttgctttctgctacgttatcccaacagcagcgcgacaaactcgtcgccgcc |
| | | tttgcgcgtggcacagagggccagcaagaagctccgttccttgaaaaggatgattacccc |
| | | tggctgacgcatgtcacgaaatccgatgtgaactcacaccgggtagcgacgcgcaaagac |
| | | gttgagcgtagcgtcagcgtgggttggcttcatagtgaacaagagagtattgcgcgtatc |
| | | gaatcggcggtaagtcagggaaaatgcatcgcctggatccggaattctgtcgatgacgct |
| | | attaaggttcatcgtcagctgcttgcccgcggcgtcattcccgcttccagcctttcactc |
| | | tttcatagccgctttgcttttagcgatcgccagcgaattgaaatggagacgctggcacgc |
| | | tttggtaaagaagacggttcacagcgtgccggaaaagtcctcatttgtactcaggtctta |
| | | gagcagagcgttgattgtgacctggacgaaatgatctccgacctggcccctgttgatttg |
| | | ctgattcagcgagcggggcgattacagcggcatatccgcgatattaatggtcagttaaag |
| | | cgtgacggaaaagacgagcgttcccctcctgaattgctgattctggcccccgtctgggac |
| | | gacgctcctggtgacgaatggttcggcagtgccatgcgtaacagtgcatatgtctatccc |
| | | gatcatggacgaatctggctgacgcagcgtgtactgcgtgagcaaggcgctattcaaatg |
| | | ccacacgcagcccgccttcttattgaatcagtctacggtgaggacgtggtaatgccggaa |
| | | ggatttgcccgcagcgagcaggagcaagtgggcaaatattactgcgatcgcgcaatggct |
| | | aaaaagtttgtcctgaacttcaagcctggctatgccgccaatatcaacgattaccttccg |
| | | gaaaagctgtcgacacgtctggctgaggaatctgtttccctgtggctggctacctgtatt |
| | | gccggtgtggtgaagccttatgccaccggtgctcacgcatgggaaatgagcgttgtcaga |
| | | gtgcgtcgaagctggtggaaaaaacatcgggatgagttttctttactggaaggggaagcg |
| | | ttcaggcagtggtgcattgaacagcggcaagatccggaaatggcaaacgtgattttagtc |
| | | actgatgacgaaagttgcgggtattcggccagggagggattgattggcaaggttgattga |
| 60 | Escherichia coli | MEPFKYICHYWGKSSKSLTKGNDIHLLIYHCLDVAAVADCWWDQSVVLQNTFCRNEMLSK |
| | Cas3 | QRVKAWLLFFIALHDIGKFDIRFQYKSAESWLKLNPATPSLNGPSTQMCRKFNHGAAGLY |
| | Strain K12 | WFNQDSLSEQSLGDFFSFFDAAPHPYESWFPWVEAVTGHHGFILHSQDQDKSRWEMPAS |
| | >ecj:JW2731 | L |
| | K07012 | ASYAAQDKQAREEWISVLEALFLTPAGLSINDIPPDCSSLLAGFCSLADWLGSWTTTNTF |
| | CRISPR- | LFNEDAPSDINALRTYFQDRQQDASRVLELSGLVSNKRCYEGVHALLDNGYQPRQLQVLV |
| | associated | DALPVAPGLTVIEAPTGSGKTETALAYAWKLIDQQIADSVIFALPTQATANAMLTRMEAS |
| | endonuclease/ | ASHLFSSPNLILAHGNSRFNHLFQSIKSRAITEQGQEEAWVQCCQWLSQSNKKVFLGQIG |
| | helicase Cas3 | VCTIDQVLISVLPVKHRFIRGLGIGRSVLIVDEVHAYDTYMNGLLEAVLKAQADVGGSVI |
| | [EC:3.1.-.- | LLSATLPMKQKQKLLDTYGLHTDPVENNSAYPLINWRGVNGAQRFDLLAHPEQLPPRFSI |
| | 3.6.4.-]\| | QPEPICLADMLPDLTMLERMIAAANAGAQVCLICNLVDVAQVCYQRLKELNNTQVDIDLF |
| | (GenBank) | HARFTLNDRREKENRVISNFGKNGKRNVGRILVATQVVEQSLDVDFDWLITQHCPADLLF |
| | ygcB; conserved | QRLGRLHRHHRKYRPAGFEIPVATILLPDGEGYGRHEHIYSNVRVMWRTQQHIEELNGAS |
| | hypothetical | LFFPDAYRQWLDSIYDDAEMDEPEWVGNGMDKFESAECEKRFKARKVLQWAEEYSLQDN |
| | protein, member | D |
| | of DEA box | ETILAVTRDGEMSLPLLPYVQTSSGKQLLDGQVYEDLSHEQQYEALALNRVNVPFTWKRS |
| | family (A) | FSEVVDEDGLLWLEGKQNLDGWVWQGNSIVITYTGDEGMTRVIPANPK |
| 61 | Escherichia coli | atggaaccttttaaatatatatgccattactggggaaaatcctcaaaaagcttgacgaaa |
| | Cas3 Nucleotide | ggaaatgatattcatctgttaatttatcattgccttgatgttgctgctgttgcagattgc |
| | sequence | tggtgggatcaatcagtcgtactgcaaaatactttttgccgaaatgaaatgctatcaaaa |
| | >ecj:JW2731 | cagagggtgaaggcctggctgttatttttcattgctcttcatgatattggaaagtttgat |
| | K07012 | atacgattccaatataaatcagcagaaagttggctgaaattaaatcctgcaacgccatca |
| | CRISPR- | cttaatggtccatcaacacaaatgtgccgtaaatttaatcatggtgcagccggtctgtat |
| | associated | tggtttaaccaggattcactttcagagcaatctctcggggattttttcagtttttttgat |
| | endonuclease/ | gccgctcctcatccttatgagtcctggtttccatgggtagaggccgttacaggacatcat |
| | helicase Cas3 | ggttttatattacattcccaggatcaagataagtcgcgttgggaaatgccagcttctctg |
| | [EC:3.1.-.- | gcatcttatgctgcgcaagataaacaggctcgtgaggagtggatatctgtactggaagca |
| | 3.6.4.-]\| | ttatttttaacgccagcggggttatctataaacgatataccacctgattgttcatcactg |
| | (GenBank) | ttagcaggtttttgctcgcttgctgactggttaggctcctggactacaacgaataccttt |
| | ygcB; conserved | ctgtttaatgaggatgcgccttccgacataaatgctctgagaacgtatttccaggaccga |
| | hypothetical | cagcaggatgcgagccgggtattggagttgagtggacttgtatcaaataagcgatgttat |
| | protein, member | gaaggtgttcatgcactactggacaatggctatcaacccagacaattacaggtgttagtt |
| | of DEAD box | gatgctcttccagtagctcccgggctgacggtaatagaggcacctacaggctccggtaaa |
| | family (N) | acggaaacagcgctggcctatgcttggaaacttattgatcaacaaattgcggatagtgtt |
| | | atttttgccctcccaacacaagctaccgcgaatgctatgcttacgagaatggaagcgagc |
| | | gcgagccacttattttcatccccaaatcttattcttgctcatggcaattcacggtttaac |
| | | cacctctttcaatcaataaaatcacgcgcgattactgaacaggggcaagaagaagcgtgg |
| | | gttcagtgttgtcagtggttgtcacaaagcaataagaaagtgtttcttgggcaaatcggc |
| | | gtttgcacgattgatcaggtgttgatatcggtattgccagttaaacaccgctttatccgt |
| | | ggtttgggaattggtcgaagtgttttaattgttgatgaagttcatgcttacgacacctat |
| | | atgaacggcttgctggaggcagtgctcaaggctcaggctgatgtgggagggagtgttatt |
| | | cttctttccgcaaccctaccaatgaaacaaaaacagaaacttctggatacttatggtctg |
| | | catacagatccagtggaaaataactccgcatatccactcattaactggcgaggtgtgaat |
| | | ggtgcgcaacgttttgatctgctagctcatccagaacaactcccgccccgcttttcgatt |
| | | cagccagaacctatttgtttagctgacatgttacctgaccttacgatgttagagcgaatg |
| | | atcgcagcggcaaacgcgggtgcacaggtctgtcttatttgcaatttggttgacgttgca |
| | | caagtatgctaccaacggctaaaggagctaaataacacgcaagtagatatagatttgttt |
| | | catgcgcgctttacgctgaacgatcgtcgtgaaaaagagaatcgagttattagcaatttc |
| | | ggcaaaaatgggaagcgaaatgttggacggatacttgtcgcaacccaggtcgtggaacaa |
| | | tcactcgacgttgattttgattggttaattactcagcattgtcctgcagatttgcttttc |
| | | caacgattgggccgtttacatcgccatcatcgcaaatatcgtcccgctggttttgagatt |
| | | cctgttgccaccattttgctgcctgatggcgagggttacggacgacatgagcatatttat |
| | | agcaacgttagagtcatgtggcggacgcagcaacatattgaggagcttaatggagcatcc |
| | | ttatttttccctgatgcttaccggcaatggctggatagcatttacgatgatgcggaaatg |
| | | gatgagccagaatgggtcggcaatggcatggataaatttgaaagcgccgagtgtgaaaaa |
| | | aggttcaaggctcgcaaggtcctgcagtgggctgaagaatatagcttgcaggataacgat |
| | | gaaaccattcttgcggtaacgagggatggggaaatgagcctgccattattgccttatgta |
| | | caaacgtcttcaggtaaacaactgctcgatggccaggtctacgaggacctaagtcatgaa |
| | | cagcagtatgaggcgcttgcacttaatcgcgtcaatgtacccttcacctggaaacgtagt |
| | | ttttctgaagtagtagatgaagatgggttactttggctggaagggaaacagaatctggat |
| | | ggatgggtctggcagggtaacagtattgttattacctatacaggggatgaagggatgacc |
| | | agagtcatccctgcaaatcccaaataa |
| 62 | Streptococcus | MKHINDYFWAKKTEENSRLLWLPLTQHLEDTKNIAGLLWEHWLSEGQKVLIENSINVKSN |
| | thermophilus | IENQGKRLAQFLGAVHDIGKATPAFQTQKGYANSVDLDIQLLEKLERAGFSGISSLQLAS |
| | Cas3 | PKKSHHSIAGQYLLSHYGVDEDIATIIGGHHGRPVDDLDGLNSQKSYPSNYYQDEKKDSL |
| | | VYQKWKSNQEAFLNWALTETGFNSVSQLPKIKQPAQVILSGLLIMSDWIASNEHFFPLLS |
| | | LDETDVKNKSQRIETGFKKWKKSNLWQPETFVDLVTLYQERFGFSPRNFQLILSQTIEKT |
| | | TNPGIVILEAPMGIGKTEAALAVSEQLSSKKGCSGLFFGLPTQATSNGIFKRIEQWTENI |
| | | KGNNSDHFSIQLVHGKAALNTDFIELLKGNTINMDDSENGSIFVNEWFSGRKTSALDDFV |
| | | VGTVDQFLMVALKQKHLALRHLGFSKKVIVIDEVHAYDAYMSQYLLEAIRWMGAYGVPVI |
| | | ILSATLPAQQREKLIKSYMAGMGVKWRDIENIDQIKIDAYPLITYNDGPDIHQVKMFEKQ |
| | | EQKNIYIHRLPEEQLFDIVKEGLDNGGVVGIIVNTVRKSQELARNFSDIFGDDMVDLLHS |
| | | NFIATERIRKEKDLLQEIGKKAIRPPKKIIIGTQVLEQSLDIDFDVLISDLAPMDLLIQR |
| | | IGRLHRHKIKRPQKHEVARFYVLGTFEEFDFDEGTRLVYGDYLLARTQYFLPDKIRLPDD |
| | | ISPLVQKVYNSDLTITFPKPELHKKYLDAKIEHDDKIKNKETKAKSYRIANPVLKKSRVR |
| | | TNSLIGWLKNLHPNDSEEKAYAQVRDIEDTVEVIALKKISDGYGLFIENKDISQNITDPI |
| | | IAKKVAQNTLRLPMSLSKAYNIDQTINELERYNNSHLSQWQNSSWLKGSLGIIFDKNNEF |
| | | ILNGFKLLYDEKYGVTIERLDKNESV |
| 63 | Streptococcus | ATGAAACATATTAATGATTATTTTTGGGCTAAGAAAACAGAGGAAAATAGTAGACTTCTT |
| | thermophilus | TGGTTACCATTAACTCAACACTTAGAAGACACGAAAAATATTGCAGGCCTCTTATGGGA |
| | Cas3 | A |
| | Nucleotide | CATTGGTTAAGTGAAGGACAAAAGGTATTAATTGAAAATTCTATTAATGTTAAATCAAAT |
| | sequence | ATTGAAAACCAAGGGAAAAGATTGGCACAATTCCTAGGAGCTGTTCATGATATCGGTAA |
| | >ENA\|HQ453272\| | A |
| | HQ453272.1 | GCAACACCAGCTTTTCAGACGCAAAAAGGTTATGCAAATTCAGTAGATTTGGATATTCAA |
| | Streptococcus | TTGTTAGAAAAATTGGAACGCGCAGGTTTTTCTGGCATTAGTTCTCTCCAACTAGCCTCC |
| | thermophilus | CCCAAAAAGAGTCATCATAGCATTGCAGGTCAATATTTGTTATCCCATTATGGCGTGGA |
| | strain | C |
| | DGCC7710 | GAAGATATTGCAACAATTATTGGTGGACACCATGGACGACCAGTTGATGATTTAGACGG |
| | CRISPR- | T |
| | associated | TTAAATTCTCAAAAAAGCTATCCCTCCAATTATTACCAGGATGAAAAGAAAGATAGTCTC |
| | nuclease/ | GTTTATCAGAAATGGAAGTCAAATCAAGAAGCTTTTTTAAACTGGGCTTTAACAGAAACA |
| | helicase | GGGTTTAATTCTGTGTCTCAGCTTCCAAAAATCAAACAGCCTGCTCAAGTTATTCTATCA |
| | (ca53) gene, | GGTTTACTCATAATGTCTGACTGGATTGCTAGTAATGAGCATTTTTTTCCTTTGTTAAGT |
| | complete cds. | TTGGATGAAACTGATGTGAAAAACAAGAGTCAACGTATTGAAACTGGGTTTAAAAAGTG |
| | | G |
| | | AAAAAATCTAACTTGTGGCAACCTGAAACTTTCGTTGACCTTGTTACTCTTTATCAGGAA |
| | | AGATTTGGATTTAGTCCACGAAATTTTCAGCTGATACTCTCACAAACAATCGAAAAGACG |
| | | ACTAATCCTGGGATAGTGATACTGGAAGCGCCAATGGGAATCGGGAAAACAGAGGCGG |
| | | CT |
| | | CTAGCGGTATCAGAGCAGTTATCTAGTAAAAAAGGATGTAGTGGATTGTTTTTTGGATTG |
| | | CCCACACAAGCAACCTCCAATGGAATTTTTAAGAGGATTGAACAGTGGACAGAGAATAT |
| | | A |
| | | AAGGGTAACAATTCTGATCATTTTTCCATTCAGCTGGTTCATGGAAAAGCAGCCTTAAAT |
| | | ACGGATTTTATTGAGTTACTTAAAGGAAATACAATTAATATGGACGACTCGGAAAACGGC |
| | | AGTATTTTTGTCAATGAGTGGTTTTCTGGGAGAAAAACTTCAGCATTAGATGATTTTGTA |
| | | GTTGGGACGGTCGACCAATTTTTAATGGTGGCTTTAAAACAAAAACATTTGGCCTTACG |
| | | T |
| | | CATTTAGGATTTAGTAAAAAAGTTATCGTTATTGATGAAGTCCACGCTTATGATGCTTAT |
| | | ATGAGCCAATATTTGTTGGAAGCTATCAGATGGATGGGAGCTTATGGTGTTCCTGTAAT |
| | | T |
| | | ATTTTATCAGCAACTTTACCTGCCCAACAAAGAGAAAAACTCATAAAAAGCTATATGGCT |
| | | GGAATGGGAGTGAAATGGCGAGATATTGAAAATATAGATCAGATAAAAATAGACGCATA |
| | | C |
| | | CCTTTAATCACTTATAATGACGGGCCTGACATTCATCAAGTTAAAATGTTCGAAAAGCAA |
| | | GAACAAAAAAATATCTACATTCATCGTTTACCAGAAGAACAGTTATTTGATATTGTAAAA |
| | | GAAGGTCTTGACAATGGTGGAGTAGTTGGGATAATTGTCAATACGGTGAGAAAATCTCA |
| | | A |
| | | GAATTGGCAAGAAATTTTTCAGATATTTTTGGAGATGATATGGTAGATTTGCTTCATTCT |
| | | AATTTCATAGCAACTGAAAGAATCCGAAAAGAAAAGGATTTATTGCAAGAAATTGGGAAA |
| | | AAAGCAATACGTCCACCAAAGAAAATCATTATTGGTACACAGGTGCTTGAACAGTCGTT |
| | | A |
| | | GATATTGATTTTGATGTACTGATAAGCGACTTAGCGCCTATGGATTTACTCATTCAACGT |
| | | ATCGGACGACTACATCGTCACAAAATCAAAAGGCCCCAAAAGCACGAAGTAGCAAGATT |
| | | T |
| | | TATGTTTTAGGAACATTTGAAGAGTTTGATTTTGATGAAGGAACGCGTTTGGTTTATGGG |
| | | GACTACCTATTAGCTAGAACTCAGTACTTTTTACCAGATAAAATACGACTTCCTGATGAT |
| | | ATTTCACCGCTAGTCCAAAAGGTTTATAATTCAGACCTAACAATTACGTTTCCAAAGCCA |
| | | GAACTTCATAAAAAATATTTGGATGCTAAAATAGAACATGATGATAAGATTAAAAATAAA |
| | | GAAACAAAGGCAAAGTCATACCGTATTGCTAATCCTGTCTTAAAAAAATCGAGAGTTCG |
| | | A |
| | | ACTAACAGTTTGATTGGTTGGTTAAAGAACCTCCATCCAAATGATAGTGAAGAAAAAGCA |
| | | TATGCTCAAGTTCGAGATATTGAAGATACAGTTGAAGTGATTGCATTAAAAAAAATATCT |
| | | GATGGGTATGGTTTGTTCATAGAAAATAAAGATATATCTCAGAACATTACTGATCCTATA |
| | | ATTGCAAAAAAGGTAGCACAAAATACTTTACGACTTCCGATGAGTTTATCCAAAGCCTAT |
| | | AATATTGATCAAACGATTAATGAGCTTGAAAGATATAACAATAGCCACTTAAGTCAATGG |
| | | CAAAACTCATCATGGTTAAAGGGATCTCTTGGGATTATTTTTGATAAAAACAATGAGTTT |
| | | ATACTGAATGGATTTAAACTATTATATGATGAAAAATATGGTGTTACCATAGAAAGGTTG |
| | | GATAAGAATGAGTCGGTTTAA |
| 64 | Salmonella | MSIYHYWGKSRRGETDGGDDYHLLCWHSLDVAAVGYWMVINNIYFIDHYLKKLGIQDKEQ |
| | enterica subsp. | AAQFFAWILCWHDIGKFAHSFQQLYRHEALNIFNEPTRHYEKIAHTTLGYMLWNSWLSEC |
| | enterica serovar | PELFPPSSLSVRKSKRVMALWMPVTTGHHGRPPEAIQELDHFRQQDKDAARDFLLRIKAL |
| | Typhimurium | FPLITLPEAWDEDEGIDQFQQLSWFISAAVVLADWTGSASRYFPRTAEKMPVDTYWQQAL |
| | LT2 Cas 3 | AKAQTAITLFPSAANVSAFTGIETLFPFIQHPTPLQQKALELDINVDGAQLFILEDVTGA |
| | | GKTEAALILAHRLMAAGKAQGLYFGLPTMATANAMFERMANTWLALYQPDSRPSLILANS |
| | | ARRLMDRFNQSIWSVTLSGTEEPDEAQPYSQGCAAWFADSNKKALLAEVGVGTLDQAMM |
| | | A |
| | | VMPFKHNNLRLLGLSNKILLADEIHACDAWMSRILEGLIERQASNGNATILLSATLSQQQ |
| | | RDKLVAAFSRGVRRSVQAPLLGHDDYPWLTQVTQTELISQRVDTRKEVERCVDIGWLHSE |
| | | EACLERIGEAVEKGNCIAWIRNSVDDAIRIYRQLQLSKVVVTENLLLFHSRFAFYDRQRI |
| | | ESQTLNLFGKQSGAQRAGKVIIATQVIEQSLDIDCDEMISDLAPVDLLIQRAGRLQRHIR |
| | | DRNGLVKKSGQDERETPVLRILAPEWDDAPRENWLSSAMRNSAYVYPDHGRMWLTQRIL |
| | | R |
| | | EQGTIRMPQSARLLIESVYGEDVNMPVGFAKTEQLQEGKFYCDRAFAGQMLLNFAPGYCA |
| | | EISDSLPEKMSTRLAEESVTLWLAKIVDSVVTPYASGEHAWEMSVLRVRQSWWNKHKDEF |
| | | EKLDGEPLRKWCAQQHQDKDFATVIVVTDFAACGYSANEGLIGMMGE |
| 65 | Salmonella | gtgtcgatatatcactattggggaaagtctcgacgaggagaaactgacggcggtgatgat |
| | enterica subsp. | taccatttgctttgctggcattctttagatgttgcggctgtgggttactggatggtgata |
| | enterica serovar | aataatatttattttattgaccactatctaaaaaaattaggcatccaggataaggagcag |
| | Typhimurium | gcggcgcaattttttgcctggattttatgttggcatgatattggaaagtttgctcattcc |
| | LT2 Cas 3 | ttccagcaactataccgtcatgaggctttaaatatctttaatgagcctacacggcattat |
| | nucleotide | gaaaaaatcgcgcataccacgctgggatacatgttgtggaactcctggctaagtgaatgc |
| | sequence | cctgaattgtttcctccttcttcgctttcagttcgtaaaagtaagcgcgttatggcgctt |
| | | tggatgccagtcactacaggtcatcatggacgccctccagaggcaatccaggagctggac |
| | | cattttcgccagcaggataaagacgcggcaagagattttcttctgagaataaaagcgctc |
| | | tttcctttaattactttgcctgaagcctgggatgaagatgagggtatcgaccaatttcag |
| | | caactttcctggtttatttccgctgcggttgtactggctgactggactggttctgccagc |
| | | cgttattttccgcgtactgcggaaaaaatgcctgttgatacctactggcagcaagctctc |
| | | gctaaagcacaaactgccatcacgctatttccctcagcggcgaatgtgtctgcctttacg |
| | | ggcatagaaacgcttttcccttttattcagcatcccacaccgttacaacaaaaggcgctt |
| | | gagctggatatcaacgtggatggcgcccaactctttattcttgaagatgtcaccggggcc |
| | | ggaaaaacagaggcggcgctcatattagctcatcgactgatggcggcaggtaaagcgcag |
| | | ggactctattttggactgccgacaatggcgacagccaacgcgatgtttgaacgtatggcg |
| | | aacacctggctggcgctgtatcagccggactcccgtcccagcctgattctggcgcatagc |
| | | gcgcgtcgcttaatggatcgtttcaatcagtcaatatggtcggtcactctttctggtacg |
| | | gaagaacccgatgaagcgcagccttatagtcagggatgcgccgcctggtttgccgacagc |
| | | aataaaaaagcgttgttggcggaggttggcgtaggcacgttggatcaggcgatgatggcg |
| | | gtaatgccatttaaacataacaacctgcggttactgggtcttagcaacaagatcttactg |
| | | gctgatgagatccatgcctgtgatgcctggatgtcccgaatacttgaaggtttgatcgaa |
| | | cggcaggccagtaatggcaacgccactattctgttatctgcgacgctatcgcagcagcag |
| | | cgagataagctggtggcggcattttcccgtggggtgaggcgtagtgtgcaggcgccgttg |
| | | ctaggccatgacgattatccctggctgactcaggtcacacaaacagagctgatttctcag |
| | | cgggttgatacacgcaaagaggttgagcgttgcgtagatattggctggctacatagtgaa |
| | | gaggcgtgtcttgaacgtataggtgaagcagtggaaaaaggaaactgtatcgcctggata |
| | | cgtaactccgttgatgatgcgattcgtatctatcgccagcttcaactgagtaaggtcgtc |
| | | gtcacggaaaaccttttactcttccatagtcgctttgctttttacgatcgtcagcggatt |
| | | gagtcacagacgctgaatctctttggcaaacagagcggcgcgcaacgtgccggtaaggtc |
| | | attatcgccacgcaggtcatcgaacaaagtctggatattgactgcgatgagatgatctct |
| | | gatttagcgccggtggatttattaattcagcgggccggtcgactacagcgtcatattcgc |
| | | gatcgtaacggtctggtgaaaaagagtgggcaggatgagcgagagacgccagtgctgcgc |
| | | attcttgctccggagtgggatgacgcgccgcgagagaactggttatccagcgccatgcgt |
| | | aacagcgcctatgtctatcccgatcatgggcgcatgtggctgacacagcgcatattacgt |
| | | gagcaggggacgattcggatgccgcaatctgcccgattgttgattgagtcggtctacggc |
| | | gaggatgtcaacatgccggttggatttgcaaaaaccgagcaattgcaggaaggcaaattt |
| | | tattgcgaccgggcatttgccggccagatgctgcttaactttgcgccgggctactgtgct |
| | | gaaattagcgattctttaccggagaaaatgtcaacgcggctggcggaagagtctgtcacg |
| | | ctgtggctggcgaaaatcgtggatagcgtcgtaaccccttatgccagcggtgaacacgcc |
| | | tgggagatgagcgtgctgcgagtacgtcagagctggtggaataaacataaagacgagttt |
| | | gaaaaattagacggcgaacccttgcgtaagtggtgtgcgcaacagcatcaggataaggat |
| | | tttgccacggtgattgtggtgacggactttgccgcttgtggttattcggcgaatgaggga |
| | | ttgattggcatgatgggggaataa |
| 66 | Escherichia coli | MNLLIDNWIPVRPRNGGKVQIINLQSLYCSRDQWRLSLPRDDMELAALALLVCIGQIIAP |
| | K-12 MG1655: | AKDDVEFRHRIMNPLTEDEFQQLIAPWIDMFYLNHAEHPFMQTKGVKANDVTPMEKLLAG |
| | 62760 CasA | VSGATNCAFVNQPGQGEALCGGCTAIALFNQANQAPGFGGGFKSGLRGGTPVTTFVRGID |
| | | LRSTVLLNVLTLPRLQKQFPNESHTENQPTWIKPIKSNESIPASSIGFVRGLFWQPAHIE |
| | | LCDPIGIGKCSCCGQESNLRYTGFLKEKFTFTVNGLWPHPHSPCLVTVKKGEVEEKFLAF |
| | | TTSAPSWTQISRVVVDKIIQNENGNRVAAVVNQFRNIAPQSPLELIMGGYRNNQASILER |
| | | RHDVLMFNQGWQQYGNVINEIVTVGLGYKTALRKALYTFAEGFKNKDFKGAGVSVHETAE |
| | | RHFYRQSELLIPDVLANVNFSQADEVIADLRDKLHQLCEMLFNQSVAPYAHHPKLISTLA |
| | | LARATLYKHLRELKPQGGPSNG |
| 67 | Escherichia coli | atgaatttgcttattgataactggatccctgtacgcccgcgaaacggggggaaagtccaa |
| | K-12 MG1655: | atcataaatctgcaatcgctatactgcagtagagatcagtggcgattaagtttgccccgt |
| | 62760 CasA | gacgatatggaactggccgctttagcactgctggtttgcattgggcaaattatcgccccg |
| | | gcaaaagatgacgttgaatttcgacatcgcataatgaatccgctcactgaagatgagttt |
| | | caacaactcatcgcgccgtggatagatatgttctaccttaatcacgcagaacatcccttt |
| | | atgcagaccaaaggtgtcaaagcaaatgatgtgactccaatggaaaaactgttggctggg |
| | | gtaagcggcgcgacgaattgtgcatttgtcaatcaaccggggcagggtgaagcattatgt |
| | | ggtggatgcactgcgattgcgttattcaaccaggcgaatcaggcaccaggttttggtggt |
| | | ggttttaaaagcggtttacgtggaggaacacctgtaacaacgttcgtacgtgggatcgat |
| | | cttcgttcaacggtgttactcaatgtcctcacattacctcgtcttcaaaaacaatttcct |
| | | aatgaatcacatacggaaaaccaacctacctggattaaacctatcaagtccaatgagtct |
| | | atacctgcttcgtcaattgggtttgtccgtggtctattctggcaaccagcgcatattgaa |
| | | ttatgcgatcccattgggattggtaaatgttcttgctgtggacaggaaagcaatttgcgt |
| | | tataccggttttcttaaggaaaaatttacctttacagttaatgggctatggccccatccg |
| | | cattccccttgtctggtaacagtcaagaaaggggaggttgaggaaaaatttcttgctttc |
| | | accacctccgcaccatcatggacacaaatcagccgagttgtggtagataagattattcaa |
| | | aatgaaaatggaaatcgcgtggcggcggttgtgaatcaattcagaaatattgcgccgcaa |
| | | agtcctcttgaattgattatggggggatatcgtaataatcaagcatctattcttgaacgg |
| | | cgtcatgatgtgttgatgtttaatcaggggtggcaacaatacggcaatgtgataaacgaa |
| | | atagtgactgttggtttgggatataaaacagccttacgcaaggcgttatatacctttgca |
| | | gaagggtttaaaaataaagacttcaaaggggccggagtctctgttcatgagactgcagaa |
| | | aggcatttctatcgacagagtgaattattaattcccgatgtactggcgaatgttaatttt |
| | | tcccaggctgatgaggtaatagctgatttacgagacaaacttcatcaattgtgtgaaatg |
| | | ctatttaatcaatctgtagctccctatgcacatcatcctaaattaataagcacattagcg |
| | | cttgcccgcgccacgctatacaaacatttacgggagttaaaaccgcaaggagggccatca |
| | | aatggctga |
| 68 | Escherichia coli | MNSFSLLTTPWLPVRFKDGTTGKLAPVDLADENVVDIAAPRADLQGAAWQFLLGLLQSSF |
| | O157 H7 | APKDYRRWDDIWEDGLEAEKLREALLSLEHPFQFGPDSPSFMQDFEVLMGDKVQVASLLP |
| | EC4115 | EIPGAQTTKFNKDHFIKRGVTEHVCSHCSALALFSLQLNAPSGGKGYRTGLRGGGPMTTL |
| | (EHEC): | IELQEYQGNQQAPLWRKLWLNVMPQDEADLPLPKKFDDLVFPWLGPTRTSELAGAVVTDD |
| | ECH74115_4013 | QVNKLQAYWGMPRRIRIDFNTTTVGNCDICGEQSDALLSLMTTKNYGANYAMWQHPLTPY |
| | Cse1 | RVPLKEGGEFYSVKPQPGGLIWRDWLGLIETGKSENNTELPALVVKLFNASSLKQAKVGL |
| | >ecf:ECH74115_ | WGFGYDFDNMKARCWYEHHFPLLLNKKEGQIPKLRLAAQTASRILSLLRSALKEAWFSDP |
| | 4013 K19123 | KGARGDFSFVDIDFWNKTQHRFLRLVRQIEEGQDADELLGKWQKEIWLFARQDFDERVFT |
| | CRISPR system | NPYEPVDLERVMTARKKYFTTSAEKQSAKAAREKKQEAAE |
| | Cascade subunit | |
| | CasA\| | |
| | (GenBank) cse1; | |
| | CRISPR- | |
| | associated | |
| | protein, Cse1 | |
| | family (A) | |
| 69 | Escherichia coli | atgaactcgttttcacttctgacaaccccgtggttgcccgttcgttttaaagacggaaca |
| | O157 H7 | acaggcaagctggcgccagtcgatctggcggatgaaaatgttgtcgatatcgctgcgccg |
| | EC4115 | cgggcagatctccagggggcggcatggcagtttttgctggggttactacaaagcagtttc |
| | (EHEC): | gcgccaaaagattatcgtcgttgggatgatatctgggaagacgggctggaagctgaaaag |
| | ECH74115_4013 | ctacgggaagcattgctgtcattagaacaccctttccagtttggcccagattcaccttca |
| | Cse1 | tttatgcaggatttcgaggtgctcatgggcgataaagttcaggtcgcttcgctactgcct |
| | >ecf:ECH74115_ | gagattcccggcgctcaaacaacgaagtttaataaagaccactttattaagcgtggcgtg |
| | 4013 K19123 | actgaacacgtatgctctcattgttctgcgttagctctgttctccctacagttaaatgcg |
| | CRISPR system | ccgtcaggtggcaaaggctatcgcaccggtttacgcggcggtgggccgatgacgactctg |
| | Cascade subunit | attgaattgcaggagtatcagggcaatcaacaagcccccttgtggcgcaaactgtggctc |
| | CasA\| | aacgtgatgccgcaggatgaagccgacttaccgctacccaaaaaatttgacgatctggtt |
| | (GenBank) cse1; | ttcccctggcttggcccgacgcgtaccagcgaactggccggtgcggtggtaaccgatgat |
| | CRISPR- | caggtcaataaactccaggcgtactggggaatgccgcggcgtattcgtattgattttaat |
| | associated | accacgacagtcggcaactgcgatatttgcggtgagcagagtgacgcgcttctgagtttg |
| | protein, Cse1 | atgactaccaaaaattacggtgcgaattatgccatgtggcagcatcccttaacgccttac |
| | family (N) | cgtgtaccacttaaagagggcggtgagttttactccgttaaaccacaaccgggcggttta |
| | | atctggcgcgactggttaggccttatcgaaacgggtaagtcagaaaacaatacggaactt |
| | | cccgcgctggtggtgaaactctttaatgccagcagtctgaaacaggcaaaagtgggcctg |
| | | tggggatttggttatgatttcgacaacatgaaagcgcgctgttggtacgaacaccatttc |
| | | ccgctgctgctcaataaaaaagaaggccagataccgaagctgcggctggctgcgcaaacg |
| | | gcttcacggattctgagtctgttacggagtgcattgaaagaagcatggttctccgatcca |
| | | aaaggtgcaaggggtgatttcagttttgtggatatcgacttctggaacaaaactcagcat |
| | | cgcttcctgaggttagtgcgccaaattgaagaaggtcaggatgcggatgaattactcggc |
| | | aaatggcaaaaggaaatttggttattcgcacgtcaggattttgacgagcgtgtattcacc |
| | | aatccttatgagcccgttgatttggaacgcgtcatgaccgcgcgcaagaaatattttaca |
| | | acatcggcggagaagcaaagtgctaaagccgccagggagaaaaagcaggaggctgctgaa |
| | | tga |
| 70 | Salmonella | MDNFSLLTTPWLPVRFKDGSTGKLAPVDLADENVVDIAATRADLQGAAWQFLLGLLQCSI |
| | enterica subsp. | APKRYKNWEDIWFDGLHADVLHKALAPLEHAFQFGAETPSFMQDFEPLSGEKVSIASLLP |
| | enterica serovar | EIPGAQTTKFNKDHFVKRGVTERFCPHCAALALFSLQLNAPAGGKGYRTGLRGGGPLTTL |
| | Typhimurium | VELQEYQGERQTPLWRKLWLNVMPQDTADLPLPDQCDATVFPWLAATRTSEQANAVTTP |
| | var. 5- | E |
| | CFSAN001921: | QVNKLQAYWGMPRRIRLDFATLQSGCCDICGAESDELLGFMTVKNYGVNYDGWRHPLTPY |
| | CFSAN001921_ | RAPVKDQNAFFSVKPQPGGLIWRDWLGLSQNNQTEANYESPAQVVKVFNARSLTDVKAGI |
| | 02360 CasA | WGFGADFDNMKIRCWYEHHFPLLMTEGLIPDLRKAVQTAARLLSLLRSALKEAWFADAKG |
| | >setc:CFSAN001921_ | ARGDFSFIDIDFWNLTQGRFLNLIHDLENGHKPDERLNKWQRELWLFTRHYFDDHVFTNP |
| | 02360 | YESSDLERIMTARKKYFTTSAEKQSAKAAKAKKQEAAE |
| | K19123 | |
| | CRISPR system | |
| | Cascade subunit | |
| | CasA\| | |
| | (GenBank) | |
| | CRISPR- | |
| | associated | |
| | protein CasA | |
| | (A) | |
| 71 | Salmonella | atggacaatttttcacttttaacaacgccctggctccccgtccgtttcaaagacggttcc |
| | enterica subsp. | acgggcaagctggcccccgtcgatctggcggatgaaaacgtggtggacatcgccgcaacg |
| | enterica serovar | cgagcagatttacagggagcggcttggcagtttctgttgggattgctgcaatgcagtatc |
| | Typhimurium | gcgccgaaaagatacaaaaattgggaggatatctggtttgatggattgcatgccgatgtg |
| | var. 5- | ctccataaggcattagcaccgttagaacacgcttttcagtttggcgcggaaacgccgtct |
| | CFSAN001921: | tttatgcaggattttgaaccgttaagcggcgaaaaagtctctattgcctcattgttgccg |
| | CFSAN001921_ | gaaatacctggcgcgcaaaccacgaagttcaataaagatcattttgtcaaacgcggcgta |
| | 02360 CasA | acggaacgtttttgtccgcactgcgcggcgctggcgctgttctcgttgcagcttaacgcg |
| | >setc:CFSAN001921_ | cctgcgggcggcaaaggctatcgtaccgggctgcgcggcggcgggccactgaccacgctg |
| | 02360 | gttgaattgcaggaatatcagggcgagcggcaaacgccgctctggcgcaagctgtggctc |
| | K19123 | aacgtgatgccgcaggatactgcggatctgcctttaccagaccagtgtgatgcgaccgtt |
| | CRISPR system | ttcccgtggcttgccgcgacgcggaccagcgagcaggcgaatgccgttaccacgccggag |
| | Cascade subunit | caggtcaataaactccaggcgtactgggggatgccgcgtcgtatccgcctggattttgcc |
| | CasA\| | accttacagtcaggttgctgcgatatttgcggcgctgaaagcgatgagcttcttggcttt |
| | (GenBank) | atgaccgtcaagaactacggcgttaactacgatggctggcggcacccgctgacgccttat |
| | CRISPR- | cgcgccccggtaaaagatcaaaacgccttcttttccgttaaaccgcagcccggcggcctt |
| | associated | atctggcgcgactggctgggattaagtcagaacaaccagacggaagcgaattacgaatct |
| | protein CasA | cccgcgcaggtagtcaaggtgtttaacgcccgctcgctgactgacgttaaagcggggatc |
| | (N) | tggggctttggcgcggatttcgacaatatgaaaatccgctgctggtatgagcatcacttc |
| | | ccgttgctgatgacggaaggtctgatccctgatttacgtaaggccgtgcaaactgcggcc |
| | | cgcctgttgagcctgcttcgcagcgcgctcaaagaggcctggtttgccgatgcgaagggt |
| | | gctcgcggtgatttcagttttatcgacattgatttctggaacctgacgcagggacgtttt |
| | | ctcaacctgattcacgatctggaaaacggccacaagccggacgaaaggctgaataaatgg |
| | | caaagagaactttggctgtttacccgtcattacttcgatgatcacgtctttaccaacccc |
| | | tacgagagcagcgatctggaacgcatcatgaccgcgcgcaagaaatattttacgacatcg |
| | | gcggaaaaacaaagtgcaaaagccgccaaagcaaagaaacaggaggctgctgaatga |
| 72 | Salmonella | MDNFSLLTTPWLPVRFKDGSTGKLAPVDLADENVVDIAATRADLQGAAWQFLLGLLQCSI |
| | enterica subsp. | APKRYKNWEDIWFDGLHADVLHKALAPLEHAFQFGAESPSFMQDFEPLSGEKVSIASLLP |
| | enterica serovar | EIPGAQTTKFNKDHFVKRGVTERFCPHCAALALFSLQLNAPAGGKGYRTGLRGGGPLTTL |
| | Enteritidis | VELQEYQGERQTPIWRKLWLNVMPQDTADLPLPDQCDATVFPWLAATRTSEQANAVTTPE |
| | EC20090193: | QVNKLQAYWGMPRRIRLDFATLQSGCCDICGAESDELLGFMTVKNYGVNYDGWRHPLTPY |
| | AU37_14140 | RAPVKDQNAFFSVKPQPGGLIWRDWLGLSQNNQTEANYESPAQVVKVFNARSLTDVKAGI |
| | CasA | RGFGADFDNMKIRCWYEHHFPLLMTEGLIPDLRKAVQTAARLLSLLRSALKEAWFTNAKD |
| | >seno:AU37_ | ARGDFSFIDIDFWNLTQGRFLNLIHDLENGHKPDERLNKWQRELWLFTRCYFDDHVFTNP |
| | 14140 K19123 | YESSDLERIMKARKKYFTSSAEKQSAKAAKAKKQEAAE |
| | CRISPR system | |
| | Cascade subunit | |
| | CasA\| | |
| | (GenBank) | |
| | CRISPR- | |
| | associated | |
| | protein CasA | |
| | (A) | |
| 73 | Salmonella | atggacaatttttcacttttaacaacgccctggctccccgtccgtttcaaagacggttcc |
| | enterica subsp. | acgggcaagctggcccccgtcgatctggcggatgaaaacgtggtggacatcgccgcaacg |
| | enterica serovar | cgagcagatttacagggagcggcctggcagtttctgttgggattgctgcaatgcagtatc |
| | Enteritidis | gcgccgaaaagatacaaaaattgggaggatatctggtttgatggattgcatgccgatgtg |
| | EC20090193: | ctccataaggcattagcaccgttagaacacgcttttcagtttggcgcggaatccccctcg |
| | AU37_14140 | tttatgcaggattttgaaccgttaagcggcgaaaaagtctctattgcctcattgttgccg |
| | CasA | gaaatacctggcgcgcaaaccacgaagttcaataaagatcattttgtcaaacgcggcgta |
| | >seno:AU37_ | acggaacgtttttgtccgcactgcgcggcgctggcgctgttctcgttgcagcttaacgcg |
| | 14140 K19123 | cctgcgggcggcaaaggctatcgtaccgggctgcgcggcggcgggccactgaccacgctg |
| | CRISPR system | gttgaattgcaggaatatcagggcgagcggcaaacgccgatctggcgcaagctgtggctc |
| | Cascade subunit | aacgtgatgccgcaggatactgcggatctgcctttaccagaccagtgtgatgcgaccgtt |
| | CasA\| | ttcccgtggcttgccgcgacgcggaccagcgagcaggcgaatgccgttaccacgccggag |
| | (GenBank) | caggtcaataaactccaggcgtactgggggatgccgcgtcgtatccgcctggattttgcc |
| | CRISPR- | accttacagtcaggttgctgcgatatttgcggcgctgaaagcgatgagcttcttggcttt |
| | associated | atgaccgtcaagaactacggcgttaactacgatggctggcggcacccgctgacgccttat |
| | protein CasA | cgcgccccggtaaaagatcaaaacgccttcttttccgttaaaccgcagcccggcggcctt |
| | (N) | atctggcgcgactggctgggattaagtcagaacaaccagacggaagcgaattacgaatct |
| | | cccgcgcaggtagtcaaggtgtttaacgcccgctcgctgactgacgttaaagcggggatc |
| | | cggggctttggcgcggatttcgacaatatgaaaatccgctgctggtatgagcatcacttc |
| | | ccgttgctgatgacggaaggtctgatccctgatttacgtaaggccgtgcaaactgcggcc |
| | | gcctgttgagcctgcttcgcagtgcgctaaaagaagcgtggttcaccaatgcgaaggat |
| | | gcgcggggtgatttcagttttatcgacattgatttctggaacctgacgcaggggcgcttt |
| | | ctcaatctgatccacgatctggaaaacggacacaagccggacgaaaggctgaataaatgg |
| | | caaagagaactttggctgtttacccgttgttacttcgatgatcacgtctttaccaacccc |
| | | tacgagagcagcgatctggagcgcatcatgaaggcgcgcaaaaaatattttacttcatcg |
| | | gcggaaaagcaaagcgcaaaagccgccaaagcaaagaaacaggaggctgctgaatga |
| 74 | S thermophilus | GTTTTTCCCGCACACGCGGGGGTGATCC |
| | CRISPR4 repeat | |

1. A nucleic acid vector for introduction into a host cell, wherein the host cell comprises a CRISPR/Cas system that is repressed by a repressor in the host cell, the vector comprising
(a) a nucleotide sequence encoding a de-repressor that is capable of de-repressing the CRISPR/Cas system in the host cell, wherein the sequence is expressible in the host cell to produce the de-repressor; and
(b) a CRISPR array for production of one or more crRNAs in the host cell; and/or one or more nucleotide sequences encoding a respective guide RNA (gRNA) in the host cell; wherein each crRNA or gRNA is capable of guiding Cas to modify a respective protospacer sequence of the host cell genome or to modify a protospacer sequence of an episome comprised by the host cell in the presence of the de-repressor.

2. The vector of claim 1, wherein the nucleotide sequence encoding the de-repressor comprises a constitutive promoter or strong promoter for expression of the nucleotide sequence in the host cell.

3. The vector of claim 1, wherein the one or more nucleotide sequences encoding the gRNA or the CRISPR array comprises a constitutive promoter or strong promoter for expression of the one or more nucleotide sequences or the CRISPR array in the host cell.

4. The vector of claim 1, wherein the nucleotide sequence encoding the de-repressor and the one or more nucleotide sequences encoding the gRNA, or the nucleotide sequence encoding the de-repressor and the CRISPR array are comprised by the same operon or under the control of a common promoter that is operable in the host cell.

5. The vector of claim 1, wherein the host cell is a wild-type host cell.

6. The vector of claim 1, wherein transcription of one or more Cas sequences is repressed.

7. The vector of claim 1, wherein Cas modification of the host cell genome or the episome comprised by the host cell
(a) kills the host cell;
(b) reduces growth or proliferation of the cell or episome;
(c) increases growth or proliferation of the cell or episome;
(d) reduces or prevents transcription of a nucleotide sequence that comprises or is adjacent a said protospacer sequence; or
(e) increases transcription of a nucleotide sequence that comprises or is adjacent a said protospacer sequence.

8. The vector of claim 1, wherein the repressor is H-NS, StpA, LRP or CRP.

9. The vector of claim 8, wherein the de-repressor is a mutant H-NS, StpA, LRP or CRP that is capable of forming a complex with H-NS, StpA, LRP or CRP repressor respectively in the host cell to prevent or reduce repression of the CRISPR/Cas system.

10. The vector of claim 1, wherein the de-repressor is LeuO or LysR or a functional equivalent thereof.

11. The vector of claim 1, wherein the cell is a bacterial or archaeal cell.

12. The vector of claim 1, wherein the vector comprises an expressible htpG sequence.

13. The vector of claim 1, wherein the host cell comprises a CRISPR/Cas system comprising Cascade and Cas3, wherein the Cascade is repressed in the host cell, wherein the vector comprises
(i) an expressible nucleotide sequence encoding a de-repressor of said Cascade repression; and
(ii) an expressible nucleotide sequence encoding a Cas3, wherein the Cas 3 is capable of functioning with de-repressed Cascade in the host cell;
wherein the nucleotide sequences of (i) and (ii) are capable of being expressed in the host cell.

14. The vector of claim 13, wherein the nucleotide sequences of (i) and (ii) are under the control of one or more constitutive promoters that is operable in the host cell.

15-16. (canceled)

17. The vector of claim 1, wherein the host cell is an E coli, Streptococcus or Salmonella cell.

18. The vector of claim 1, wherein the protospacer sequence is a chromosomal sequence, an endogenous host cell sequence, a wild-type host cell sequence, a non-viral chromosomal host cell sequence, and/or a non-phage sequence.

19. The vector of claim 1, wherein the CRIPSR/Cas system comprises a Cas3 and a repressed Cascade and the de-repressor is capable of de-repressing the Cascade in the host cell, wherein the one or more nucleotide sequences or the CRISPR array comprises a CRISPR repeat sequence that is operable with the de-repressed CRISPR/Cas system, and wherein the repeat sequence comprising or consisting of a sequence selected from SEQ ID NOs: 49-52, or a sequence that is at least 70% identical to said selected sequence.

20. The vector of claim 19, wherein:
(i) the Cas3 comprises the amino acid sequence of SEQ ID NO: 58 or 60, or an amino acid sequence that is at least 70% identical to SEQ ID NO: 58 or 60;
(ii) the Cas3 is operable with a PAM comprising or consisting of the nucleotide sequence AWG; and/or
(iii) the Cascade comprises a repressed CasA and the de-repressor is capable of de-repressing the CasA, the CasA comprising the amino acid sequence of SEQ ID NO: 66 or 68, or an amino acid sequence that is at least 70% identical to SEQ ID NO: 66 or 68.

21-22. (canceled)

23. The vector of claim 1, wherein the host cell is a S. enterica cell and the CRISPR/Cas system comprises a type E (Cse) Cas that is repressed, wherein the de-repressor is capable of de-repressing the Cas.

24. The vector of claim 1, wherein the CRIPSR/Cas system comprises a Cas3 and a repressed Cascade and the de-repressor is capable of de-repressing the Cascade in the host cell, wherein the one or more nucleotide sequences or the CRISPR array comprises a CRISPR repeat sequence that is operable with the de-repressed CRISPR/Cas system, the repeat sequence comprising or consisting of SEQ ID NO: 53, or a sequence that is at least 70% identical thereto.

25-26. (canceled)

27. The vector of claim 1, wherein the one or more nucleotide sequences or the CRISPR array comprises a CRISPR repeat sequence that is operable with the de-repressed CRISPR/Cas system in the host cell, the repeat sequence being at least 90% identical to a repeat in a host array comprised by the CRISPR/Cas system of the host cell, wherein the vector or the one or more nucleotide sequences or the CRISPR array does not comprise a PAM recognized by a Cas nuclease of the host CRISPR/Cas system.

28. The vector of, wherein:
(i) the vector comprises no sequences from the group consisting of CasA, B, C, D and E nucleotide sequences, or wherein the vector does not comprise all of the sequences of said group;
(ii) the vector comprises no sequences from the group consisting of Cas1, Cas2, Cas5 and Cas6 sequences; and/or
(iii) the vector comprises no Cas 3 nucleotide sequence.

29-33. (canceled)

34. A plurality of bacteriophage or phagemids comprising a plurality of vectors of claim 1.

35.-54. (canceled)

55. The vector of claim 8, wherein the de-repressor is LeuO or LysR or a functional equivalent thereof.

56. The vector of claim 55, wherein the de-repressor is LeuO or a functional equivalent thereof.

57. The vector of claim 23, wherein the de-repressor is LeuO or a functional equivalent thereof.


SEQUENCE LISTING (74 sequences)

SEQ ID NO: 1 - DNA, 30 bp, Artificial Sequence
  UP-IGLB
ttgttctccttcatatgctccgacatttct

SEQ ID NO: 2 - DNA, 30 bp, Artificial Sequence
  DOWN-IGLB
cttcgggaatgattgttatcaatgacgata

SEQ ID NO: 3 - protein, 314 aa, Artificial Sequence
  Escherichia coli K-12 MG1655 LeuO
MPEVQTDHPETAELSKPQLRMVDLNLLTVFDAVMQEQNITRAAHVLGMSQPAVSNAVARLKVMFNDELFVRYGRGIQPTARAFQLFGSVRQALQLVQNELPGSGFEPASSERVFHLCVCSPLDSILTSQIYNHIEQIAPNIHVMFKSSLNQNTEHQLRYQETEFVISYEDFHRPEFTSVPLFKDEMVLVASKNHPTIKGPLLKHDVYNEQHAAVSLDRFASFSQPWYDTVDKQASIAYQGMAMMSVLSVVSQTHLVAIAPRWLAEEFAESLELQVLPLPLKQNSRTCYLSWHEAAGRDKGHQWMEEQLVSICKR

SEQ ID NO: 4 - DNA, 945 bp, Artificial Sequence
  Escherichia coli K-12 MG1655 LeuO
atgccagaggtacaaacagatcatccagagacggcggagttaagcaaaccacagctacgcatggtcgatctcaacttattaaccgttttcgatgccgtgatgcaggagcaaaacattactcgtgccgctcatgttctgggaatgtcgcaacctgcggtcagtaacgctgttgcacgcctgaaggtgatgtttaatgacgagctttttgttcgttatggccgtggtattcaaccgactgctcgcgcatttcaactttttggttcagttcgtcaggcattgcaactagtacaaaatgaattgcctggttcaggttttgaacccgcgagcagtgaacgtgtatttcatctttgtgtttgcagcccgttagacagcattctgacctcgcagatttataatcacattgagcagattgcgccaaatatacatgttatgttcaagtcttcattaaatcagaacactgaacatcagctgcgttatcaggaaacggagtttgtgattagttatgaagacttccatcgtcctgaatttaccagcgtaccattatttaaagatgaaatggtgctggtagccagcaaaaatcatccaacaattaagggcccgttactgaaacatgatgtttataacgaacaacatgcggcggtttcgctcgatcgtttcgcgtcatttagtcaaccttggtatgacacggtagataagcaagccagtatcgcgtatcagggcatggcaatgatgagcgtacttagcgtggtgtcgcaaacgcatttggtcgctattgcgccgcgttggctggctgaagagttcgctgaatccttagaattacaggtattaccgctgccgttaaaacaaaacagcagaacctgttatctctcctggcatgaagctgccgggcgcgataaaggccatcagtggatggaagagcaattagtctcaatttgcaaacgctaa

SEQ ID NO: 5 - protein, 320 aa, Artificial Sequence
  Escherichia coli O157 H7 EDL933 (EHEC) LeuO
MTVELSMPEVQTDHPETAEFSKPQLRMVDLNLLTVFDAVMQEQNITRAAHVLGMSQPAVSNAVARLKVMFNDELFVRYGRGIQPTARAFQLFGSVRQALQLVQNELPGSGFEPASSERVFHLCVCSPLDSILTSQIYNHIEQIAPNIHVMFKSSLNQNTEHQLRYQETEFVISYEDFHRPEFTSVPLFKDEMVLVASKNHPTIKGPLLKHDVYNEQHAAVSLDRFASFSQPWYDTVDKQASIAYQGMAMMSVLSVVSQTHLVAIAPRWLAEEFAESLELQVLPLPLKLNSRTCYLSWHEAAGRDKGHQWMEEQLVSICKR

SEQ ID NO: 6 - DNA, 963 bp, Artificial Sequence
  Escherichia coli O157 H7 EDL933 (EHEC) LeuO
gtgacagtggagttaagtatgccagaggtacaaacagatcatccagagacggcggagttcagcaagccacagctacgcatggtcgatctcaacttattaaccgttttcgatgccgtgatgcaggagcaaaacattacccgtgctgctcatgttctgggaatgtcgcaacctgcggtcagtaacgctgttgcacgcctgaaggtgatgtttaatgacgagctttttgttcgttatggccgtggtattcaaccgactgctcgcgcatttcaactttttggttcagttcgtcaggcattgcaactagtacaaaatgaattgcctggttcaggttttgaacccgcgagcagtgaacgtgtatttcatctttgtgtttgcagcccgttagacagtattctgacctcgcagatttataatcacattgagcagattgcgccaaatatacatgttatgttcaagtcttcattaaatcagaacactgaacatcagctgcgttatcaggaaacggagtttgtgattagttatgaagacttccatcgtcctgaatttaccagcgtgccattatttaaagatgaaatggtgctggtagccagcaaaaatcacccaacaattaagggcccgttactgaaacatgatgtttataacgaacaacatgcggcggtttcgctcgatcgtttcgcgtcatttagtcaaccttggtatgacacggtagataagcaagccagtatcgcgtatcagggcatggcaatgatgagcgtacttagcgtggtgtcgcaaacgcatttggtcgctattgcgccgcgttggctggctgaagagttcgctgaatccttagaattacaggtattaccgctgccgttaaaactaaatagcagaacctgttatctctcctggcatgaagctgccgggcgtgataaaggccatcagtggatggaagagcaattagtctcaatttgcaaacgctaa

SEQ ID NO: 7 - protein, 314 aa, Artificial Sequence
  Salmonella enterica subsp. enterica serovar Typhi CT18 STY0134 LeuO
MPEVKTEKPHLLDMGKPQLRMVDLNLLTVFDAVMQEQNITRAAHTLGMSQPAVSNAVARLKVMFNDELFVRYGRGIQPTARAFQLFGSVRQALQLVQNELPGSGFEPTSSERVFNLCVCSPLDNILTSQIYNRVEKIAPNIHVVFKASLNQNTEHQLRYQETEFVISYEEFRRPEFTSVPLFKDEMVLVASRKHPRISGPLLEGDVYNEQHAVVSLDRYASFSRPWYDTPDKQSSVAYQGMALISVLNVVSQTHLVAIAPCWLAEEFAESLELQILPLPLKLNSRTCYLSWHEAAGRDKGHQWMEDLLVSVCKR

SEQ ID NO: 8 - DNA, 945 bp, Artificial Sequence
  Salmonella enterica subsp. enterica serovar Typhi CT18 STY0134 LeuO
atgccagaggtcaaaaccgaaaagccgcatcttttagatatgggcaaaccacagcttcgcatggttgatttgaacctattgaccgtgttcgatgcggtaatgcaagagcagaatattacgcgcgccgcccacacgctgggaatgtcgcagcctgcggtcagtaacgccgtagcgcgtctgaaggttatgtttaatgacgaactttttgttcgatatggacgaggaattcagccgactgcccgtgcatttcagttatttggttcagtccgtcaggcgttgcaattggtgcaaaatgaattgccgggatcggggtttgagccgaccagcagcgaacgtgtattcaatctttgcgtgtgcagtccgctggataatatcctgacgtcacagatttataatcgtgtagaaaaaattgcgccaaatattcatgtcgtttttaaagcgtcgttgaatcagaatactgagcatcagttacgctatcaggaaaccgagttcgttattagttatgaagaattccgtcgtcctgagtttaccagcgtaccgctatttaaagatgaaatggttttagtcgccagccgaaaacacccgcgtattagcggcccgctactggaaggcgatgtttataatgaacaacatgcggttgtttctctcgatcgttatgcgtcatttagtcggccgtggtatgacacgccggataaacagtcgagcgtggcttatcagggcatggcgcttatcagcgttctgaacgtggtttcgcagacgcatttggtcgctattgccccgtgctggctggcggaagagtttgcggagtcgctggagctgcaaatactgccgttgcctttaaaactgaatagccggacatgctacctttcctggcatgaagcggctgggcgtgataaagggcatcaatggatggaagatttattagtctctgtttgtaagcgataa

SEQ ID NO: 9 - protein, 314 aa, Artificial Sequence
  Salmonella enterica subsp. enterica serovar Typhimurium LT2 STM0115 LeuO
MPEVKTEKPHLLDMGKPQLRMVDLNLLTVFDAVMQEQNITRAAHTLGMSQPAVSNAVARLKVMFNDELFVRYGRGIQPTARAFQLFGSVRQALQLVQNELPGSGFEPTSSERVFNLCVCSPLDNILTSQIYNRVEKIAPNIHVVFKASLNQNTEHQLRYQETEFVISYEEFRRPEFTSVPLFKDEMVLVASRKHPRISGPLLEGDVYNEQHAVVSLDRYASFSQPWYDTPDKQSSVAYQGMALISVLNVVSQTHLVAIAPRWLAEEFAESLDLQILPLPLKLNSRTCYLSWHEAAGRDKGHQWMEDLLVSVCKR

SEQ ID NO: 10 - DNA, 945 bp, Artificial Sequence
  Salmonella enterica subsp. enterica serovar Typhimurium LT2 STM0115 LeuO
atgccagaggtcaaaaccgaaaagccgcatcttttagatatgggcaaaccacagcttcgcatggttgatttgaacctattgaccgtgttcgatgcggtaatgcaagagcagaatattacgcgcgccgcccacacgctgggaatgtcgcagcctgcggtcagtaacgccgtagcgcgtctgaaggttatgtttaatgacgaactttttgttcgatatggacgaggaattcagccgactgcccgtgcatttcagttatttggttcagtccgtcaggcgttgcaattggtgcaaaatgaattgccgggatcggggtttgagccgaccagcagcgaacgtgtattcaatctttgcgtgtgcagtccgctggataatatcctgacgtcacagatttataatcgtgtagaaaaaattgcgccaaatattcatgtcgtttttaaagcgtcgttgaatcagaatactgagcatcagttacgctatcaggaaaccgagttcgttattagttatgaagaattccgtcgtcctgagtttaccagcgtaccgctatttaaagatgaaatggttttagtcgccagccgaaaacacccgcgtattagcggcccgctactggaaggcgatgtttataatgaacaacatgcggttgtttccctcgatcgttatgcgtcatttagtcagccgtggtatgacacgccggataaacagtcgagcgtggcttatcagggcatggcgcttatcagcgttctgaacgtggtttcgcagacgcatttggtcgctattgccccgcgctggctggcggaagagtttgcggaatcgctggatctgcaaatattgccgttgcctttaaaactgaatagccggacatgctacctttcctggcatgaagcggctgggcgtgataaagggcatcaatggatggaagatttattagtctctgtttgtaagcgataa

SEQ ID NO: 11 - protein, 314 aa, Artificial Sequence
  Salmonella enterica subsp. enterica serovar Paratyphi A ATCC9150 SPA0117 LeuO
MPEVKTEKPHLLDMGKPQLRMVDLNLLTVFDAVMQEQNITRAAHTLGMSQPAVSNAVARLKVMFNDELFVRYGRGIQPTARAFQLFGSVRQALQLVQNELPGSGFEPTSSERVFNLCVCSPLDNILTSQIYNRVEKIAPNIHVVFKASLNQNTEHQLRYQETEFVISYEEFRRPEFTSVPLFKDEMVLVASRKHPRISGPLLEGDVYNEQHAVVSLDRYASFSQPWYDTPDKQSSVAYQGMALISVLNVVSQTHLVAIAPRWLAEEFAESLELQILPLPLKLNSRTCYLSWHEAAGRDKGHQWMEDLLVSVCKR

SEQ ID NO: 12 - DNA, 945 bp, Artificial Sequence
  Salmonella enterica subsp. enterica serovar Paratyphi A ATCC9150 SPA0117 LeuO
atgccagaggtcaaaaccgaaaagccgcatcttttagatatgggcaaaccacagcttcgcatggttgatttgaacctattgaccgtgttcgatgcggtaatgcaagagcagaatattacgcgcgccgcccacacgctgggaatgtcgcagcctgcggtcagtaacgccgtagcgcgtctgaaggttatgtttaatgacgaactttttgttcgatatggacgaggaattcagccgactgcccgtgcatttcagttatttggttcagtccgtcaggcgttgcaattggtgcaaaatgaattgccgggatcagggtttgagccgaccagcagcgaacgtgtattcaatctttgcgtgtgcagtccgctggataatatcctgacgtcacagatttataatcgtgtagaaaaaattgcgccaaatattcatgtcgtttttaaagcgtcgttgaatcagaatactgagcatcagttacgctatcaggaaaccgagttcgttattagttatgaagaattccgtcgtcctgagtttaccagcgtaccgctatttaaagatgaaatggttttagtcgccagccgaaaacacccgcgtattagcggcccgctactggaaggcgatgtttataatgaacaacatgcggttgtttctctcgatcgttatgcgtcatttagtcagccgtggtatgacacgccggataaacagtcgagcgtggcttatcagggcatggcgcttatcagcgttctgaacgtggtttcgcagacgcatttggtcgctattgccccgcgctggctggcggaagagtttgcggagtcgctggagctgcaaatactgccgttgcctttaaaactgaatagccggacatgctacctttcctggcatgaagcggctgggcgtgataaagggcatcaatggatggaagatttattagtttctgtttgtaagcgataa

SEQ ID NO: 13 - protein, 314 aa, Artificial Sequence; identical to SEQ ID NO: 9
  Salmonella enterica subsp. enterica serovar Enteritidis OLF-SE1-1019-1 IY59_00600 LeuO

SEQ ID NO: 14 - DNA, 945 bp, Artificial Sequence
  Salmonella enterica subsp. enterica serovar Enteritidis OLF-SE1-1019-1 IY59_00600 LeuO
atgccagaggtcaaaaccgaaaagccgcatcttttagatatgggcaaaccacagcttcgcatggttgatttgaacctattgaccgtgttcgatgcggtaatgcaagagcagaatattacgcgcgccgcccacacgctgggaatgtcgcagcctgcggtcagtaacgccgtagcgcgtctgaaggttatgtttaatgacgaactttttgttcgatatggacgaggaattcagccgactgcccgtgcatttcagttatttggttcagtccgtcaggcgttacaattggtgcaaaatgaattgccgggatcggggtttgagccgaccagcagcgaacgtgtattcaatctttgcgtgtgcagtccgctggataatatcctgacgtcacagatttataatcgtgtagaaaaaattgcgccaaatattcatgtcgtttttaaagcgtcgttgaatcagaatactgagcatcagttacgctatcaggaaaccgagttcgttattagttatgaagaattccgtcgtcctgagtttaccagcgtaccgctatttaaagatgaaatggttttagtcgccagccgaaaacacccgcgtattagcggcccgctactggaaggcgatgtttataatgaacaacatgcggttgtttctctcgatcgttatgcgtcatttagtcagccgtggtatgacacgccggataaacagtcgagcgtggcttatcagggcatggcgcttatcagcgttctgaacgtggtttcgcagacgcatttggtcgctattgccccgcgctggctggcggaagagtttgcggaatcgctggatctgcaaatattgccgttgcctttaaaactgaatagccggacatgctacctttcctggcatgaagcggctgggcgtgataaagggcaccaatggatggaagatttattagtttctgtttgtaagcgataa

SEQ ID NO: 15 - protein, 373 aa, Artificial Sequence
  Shigella flexneri 301 (serotype 2a) SF0071
MTHSTAMDSVFIRTRIFMFSEFYSFCFFLFYMHDKSYSSGLFLCIPIRERELSVTVELSMPEVQTDHSETAELSKPQLRMVDLNLLTVFDAVMQEQNITRAAHVLGMSQPAVSNAVARLKVMFNDELFVRYGRGIQPTARAFQLFGSVRQALQLVQNELPGSGFEPASSERVFHLCVCSPLDSILTSQIYNHIEQIAPNIHVMFKSSLNQNTEHQLRYQETEFVISYEDFHRPEFTSVPLFKDEMVLVASKNHPTIKGPLLKHDVYNEQHAAVSLDRFASFSQPWYDTVDKQASIAYQGMAMMSVLSVVSQTHLVAIAPRWLAEEFAESLELQVLPLPLKQNSRTCYLSWHEAAGRDKGHQWMEEQLVSICKR

SEQ ID NO: 16 - DNA, 1122 bp, Artificial Sequence
  Shigella flexneri 301 (serotype 2a) SF0071
atgactcattccacggcaatggattctgtttttatcagaacccgtatctttatgttttccgaattttactcattttgctttttcttattttatatgcatgataaatcatattcttcaggattatttctctgcattccaataagggaaagggagttaagtgtgacagtggagttaagtatgccagaggtacaaacagatcattcagagacggcggagttaagcaagccacagctacgcatggtcgatctcaacttattaaccgttttcgatgccgtgatgcaggagcaaaacattacccgtgccgctcatgttctgggtatgtcgcaacctgcggtcagtaacgctgttgcacgcctgaaggtgatgtttaatgacgagctttttgttcgttatggccgtggtattcaaccgactgctcgcgcatttcaactttttggttcagttcgccaggcattgcaactagtacaaaatgaattgcctggttcaggttttgaacccgcgagcagtgaacgtgtatttcatctttgtgtttgcagcccgttagacagcattctgacctcgcagatttataatcacattgagcagattgcgccaaatatacatgttatgttcaagtcttcattaaatcagaacactgaacatcagctgcgttatcaggaaacggagtttgtgattagttatgaagacttccatcgtcctgaatttaccagcgtgccattatttaaagatgaaatggtgctggtagccagcaaaaatcatccaacaattaaaggcccgttactgaaacatgatgtttataacgaacaacatgcggcggtttcgctcgatcgtttcgcgtcatttagtcaaccttggtatgacacggtagataagcaagccagtatcgcgtatcagggcatggcaatgatgagcgtacttagcgtggtgtcgcaaacgcatttggtcgctattgcgccgcgttggctggctgaagagttcgctgaatccttagaattacaggtattaccgctgccgttaaaacaaaacagcagaacctgttatctctcttggcatgaagctgccgggcgcgataaaggccatcagtggatggaagaacaattagtctcaatttgcaaacgctaa

SEQ ID NO: 17 - protein, 137 aa, Artificial Sequence
  Escherichia coli O157 H7 EDL933 (EHEC) Z2013 H-NS
MSEALKILNNIRTLRAQARECTLETLEEMLEKLEVVVNERREEESAAAAEVEERTRKLQQYREMLIADGIDPNELLNSLAAVKSGTKAKRAQRPAKYSYVDENGETKTWTGQGRTPAVIKKAMDEQGKSLDDFLIKQ

SEQ ID NO: 18 - DNA, 414 bp, Artificial Sequence
  Escherichia coli O157 H7 EDL933 (EHEC) Z2013 H-NS
atgagcgaagcacttaaaattctgaacaacatccgtactcttcgtgcgcaggcaagagaatgtacacttgaaacgctggaagaaatgctggaaaaattagaagttgttgttaacgaacgtcgcgaagaagaaagcgcggctgctgctgaagttgaagagcgcactcgtaaactgcagcaatatcgcgaaatgctgatcgctgacggtattgacccgaacgagctgctgaatagccttgccgccgttaaatctggcaccaaagctaaacgtgctcagcgtccggcaaaatatagctacgttgacgaaaacggcgaaactaaaacctggactggccagggccgtactccagctgtaatcaaaaaagcaatggatgagcaaggtaaatccctcgacgatttcctgatcaagcaataa

SEQ ID NO: 19 - protein, 137 aa, Artificial Sequence; identical to SEQ ID NO: 17
  Escherichia coli O127 H6 E2348/69 (EPEC) E2348C_1364 H-NS

SEQ ID NO: 20 - DNA, 414 bp, Artificial Sequence
  Escherichia coli O127 H6 E2348/69 (EPEC) E2348C_1364 H-NS
atgagcgaagcacttaaaattctgaacaacatccgtactcttcgtgcgcaggcaagagaatgtacacttgaaacgctggaagaaatgctggaaaaattagaagttgttgttaacgaacgtcgcgaagaagaaagcgcggctgctgctgaagttgaagagcgcactcgtaaactgcagcaatatcgcgaaatgctgatcgctgacggtattgacccgaacgaactgctgaatagccttgctgccgttaaatctggcaccaaagctaagcgtgctcagcgtccggcaaaatatagctacgttgacgaaaacggcgaaactaaaacctggactggccagggccgtactccagctgtaatcaaaaaagcaatggatgagcaaggtaaatccctcgacgatttcctgatcaagcaataa

SEQ ID NO: 21 - protein, 137 aa, Artificial Sequence
  Salmonella enterica subsp. enterica serovar Typhi CT18 STY1299 H-NS
MSEALKILNNIRTLRAQARECTLETLEEMLEKLEVVVNERREEESAAAAEVEERTRKLQQYREMLIADGIDPNELLNSMAAAKSGTKAKRAARPAKYSYVDENGETKTWTGQGRTPAVIKKAMEEQGKQLEDFLIKE

SEQ ID NO: 22 - DNA, 414 bp, Artificial Sequence
  Salmonella enterica subsp. enterica serovar Typhi CT18 STY1299 H-NS
atgagcgaagcacttaaaattctgaacaacatccgtactcttcgtgcgcaggcaagagaatgtactctggaaacgcttgaagaaatgctggaaaaattagaagttgtcgttaatgagcgtcgtgaagaagaaagcgctgctgctgctgaagtggaagaacgcactcgtaaactgcaacagtatcgtgaaatgttaattgccgacggcattgacccgaatgaactgctgaatagcatggctgccgctaaatccggtaccaaagctaaacgcgcagctcgtccggctaaatatagctatgttgacgaaaacggtgaaactaaaacctggactggccagggtcgtacaccggctgtaatcaaaaaagcaatggaagaacaaggtaagcaactggaagatttcctgatcaaggaataa

SEQ ID NO: 23 - protein, 137 aa, Artificial Sequence; identical to SEQ ID NO: 21
  Salmonella enterica subsp. enterica serovar Typhimurium LT2 STM1751 H-NS

SEQ ID NO: 24 - DNA, 414 bp, Artificial Sequence; identical to SEQ ID NO: 22
  Salmonella enterica subsp. enterica serovar Typhimurium LT2 STM1751 H-NS

SEQ ID NO: 25 - protein, 137 aa, Artificial Sequence; identical to SEQ ID NO: 21
  Salmonella enterica subsp. enterica serovar Enteritidis EC20090193 AU37_06605 H-NS

SEQ ID NO: 26 - DNA, 414 bp, Artificial Sequence; identical to SEQ ID NO: 22
  Salmonella enterica subsp. enterica serovar Enteritidis EC20090193 AU37_06605 H-NS

SEQ ID NO: 27 - protein, 137 aa, Artificial Sequence; identical to SEQ ID NO: 17
  Shigella flexneri 2457T (serotype 2a) S1323 H-NS

SEQ ID NO: 28 - DNA, 414 bp, Artificial Sequence
  Shigella flexneri 2457T (serotype 2a) S1323 H-NS
atgagcgaagcacttaaaattctgaacaacatccgtactcttcgtgcgcaggcaagagaatgtacacttgaaacgctggaagaaatgctggaaaaattagaagttgttgttaacgaacgtcgcgaagaagaaagcgcggctgctgctgaagttgaagagcgcactcgtaagctgcagcaatatcgcgaaatgctgatcgctgacggtattgacccgaacgaactgctgaatagccttgctgccgttaaatctggcaccaaagctaaacgtgctcagcgtccggcaaaatatagctacgttgacgaaaacggcgaaactaaaacctggactggccaaggccgtactccagctgtaatcaaaaaagcaatggatgagcaaggtaaatccctcgacgatttcctgatcaagcaataa

SEQ ID NO: 29 - protein, 134 aa, Artificial Sequence
  Escherichia coli K-12 MG1655 b2669 StpA
MSVMLQSLNNIRTLRAMAREFSIDVLEEMLEKFRVVTKERREEEEQQQRELAERQEKISTWLELMKADGINPEELLGNSSAAAPRAGKKRQPRPAKYKFTDVNGETKTWTGQGRTPKPIAQALAEGKSLDDFLI

SEQ ID NO: 30 - DNA, 405 bp, Artificial Sequence
  Escherichia coli K-12 MG1655 b2669 StpA
atgtccgtaatgttacaaagtttaaataacattcgcaccctccgtgcgatggctcgcgaattctccattgacgttcttgaagaaatgctcgaaaaattcagggttgtcactaaagaaagacgtgaagaagaagaacagcagcagcgtgaactggcagagcgccaggaaaaaattagcacctggctggagctgatgaaagctgacggaattaacccggaagagttattgggtaatagctctgctgctgcaccacgcgctggtaaaaaacgccagccgcgtccggcgaaatataaattcaccgatgttaacggtgaaactaaaacctggaccggtcagggccgtacaccgaagccaattgctcaggcgctggcagaaggtaaatctctcgacgatttcctgatctaa

SEQ ID NO: 31 - protein, 133 aa, Artificial Sequence
  Salmonella enterica subsp. enterica serovar Typhimurium LT2 STM2799 StpA
MNLMLQNLNNIRTLRAMAREFSIDVLEEMLEKFRVVTKERREEEELQQRQLAEKQEKINAFLELMKADGINPEELFAMDSAMPRSAKKRQPRPAKYRFTDFNGEEKTWTGQGRTPKPIAQALAAGKSLDDFLI

SEQ ID NO: 32 - DNA, 402 bp, Artificial Sequence
  Salmonella enterica subsp. enterica serovar Typhimurium LT2 STM2799 StpA
atgaatttgatgttacagaacttaaataatatccgcacgctgcgcgctatggctcgcgaattctccattgacgttcttgaagaaatgctcgaaaaattcagggttgtcactaaagaaagacgcgaagaagaagaattgcagcaacgccagcttgccgagaagcaggagaaaattaatgcctttctggagctgatgaaagcagacggtattaacccggaagagttatttgccatggattcagcaatgccgcgttctgctaaaaagcgccagccgcgtccggcaaaatatcgttttactgatttcaatggcgaagaaaaaacctggaccggacaaggtcgtacgcctaaaccgattgcccaggcgctggcggcggggaaatctctggatgatttcttaatctaa

SEQ ID NO: 33 - protein, 133 aa, Artificial Sequence; identical to SEQ ID NO: 31
  Salmonella enterica subsp. enterica serovar Typhimurium UK-1 STMUK_2788 StpA

SEQ ID NO: 34 - DNA, 402 bp, Artificial Sequence; identical to SEQ ID NO: 32
  Salmonella enterica subsp. enterica serovar Typhimurium UK-1 STMUK_2788 StpA

SEQ ID NO: 35 - protein, 134 aa, Artificial Sequence; identical to SEQ ID NO: 29
  Shigella flexneri 2457T (serotype 2a) S2883 StpA

SEQ ID NO: 36 - DNA, 405 bp, Artificial Sequence
  Shigella flexneri 2457T (serotype 2a) S2883 StpA
atgtccgtaatgttacaaagtttaaataacattcgcaccctccgtgcgatggctcgcgaattctccattgacgttcttgaagaaatgctcgaaaaattcagggttgtcactaaagaaagacgtgaagaagaagaacagcagcagcgtgaactggctgagcgtcaggaaaaaattagcacctggctggagctgatgaaagctgacggaattaacccggaagagttattgggtaatagctctgctgctgcaccacgtgctggtaaaaaacgccagccgcgtccggcgaaatataaattcactgatgttaacggtgaaactaaaacctggaccggtcagggccgtacaccgaagccaattgctcaggcgctggcagaaggtaaatctctcgacgatttcctgatctaa

SEQ ID NO: 37 - protein, 164 aa, Artificial Sequence
  Escherichia coli K-12 MG1655 b0889 LRP
MVDSKKRPGKDLDRIDRNILNELQKDGRISNVELSKRVGLSPTPCLERVRRLERQGFIQGYTALLNPHYLDASLLVFVEITLNRGAPDVFEQFNTAVQKLEEIQECHLVSGDFDYLLKTRVPDMSAYRKLLGETLLRLPGVNDTRTYVVMEEVKQSNRLVIKTR

SEQ ID NO: 38 - DNA, 495 bp, Artificial Sequence
  Escherichia coli K-12 MG1655 b0889 LRP
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcatttagtatccggtgatttcgactacctgttgaaaacacgcgtgccggatatgtcagcctaccgtaagttgctgggggaaaccctgctgcgtctgcctggcgtcaatgacacacggacatacgttgttatggaagaagtcaagcagagtaatcgtctggttattaagacgcgctaa

SEQ ID NO: 39 - protein, 164 aa, Artificial Sequence
  Salmonella enterica subsp. enterica serovar Typhimurium DT104 DT104_09341 LRP
MVDSKKRPGKDLDRIDRNILNELQKDGRISNVELSKRVGLSPTPCLERVRRLERQGFIQGYTALLNPHYLDASLLVFVEITLNRGAPDVFEQFNAAVQKLEEIQECHLVSGDFDYLLKTRVPDMSAYRKLLGETLLRLPGVNDTRTYVVMEEVKQSNRLVIKTR

SEQ ID NO: 40 - DNA, 495 bp, Artificial Sequence
  Salmonella enterica subsp. enterica serovar Typhimurium DT104 DT104_09341 LRP
atggtagatagcaagaagcgccctggcaaagatctcgaccgtatcgatcgtaacattcttaatgaactgcaaaaggatgggcgtatttccaacgtcgagctttctaaacgagtaggactttcgccgacaccttgccttgagcgtgtgcgtcggctggagcgacaggggtttatccagggctatacggcgctgttgaacccgcattatctggatgcgtcacttctggtattcgttgagattaccttaaatcgcggcgcgccggatgtgtttgaacagtttaatgccgccgtgcaaaagcttgaagagattcaggagtgtcatttggtttccggcgatttcgactacctgttgaaaacccgtgtaccggatatgtcagcgtatcgaaaactattgggagagacgttgctgcgcttgccaggtgtgaacgacacccgaacttacgtagtgatggaagaggtaaaacagagtaatcgtctggttattaagacacgctaa

SEQ ID NO: 41 - protein, 164 aa, Artificial Sequence; identical to SEQ ID NO: 37
  Shigella flexneri 2457T (serotype 2a) S0889 LRP

SEQ ID NO: 42 - DNA, 495 bp, Artificial Sequence
  Shigella flexneri 2457T (serotype 2a) S0889 LRP
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcatttagtatctggtgatttcgactacctgttgaaaacacgcgtgccggatatgtcagcttaccgtaagttgctgggggaaaccctgctgcgtctgcctggcgtcaatgacacacggacatacgttgttatggaagaagtcaagcagagtaatcgtctggttattaagacgcgctaa

SEQ ID NO: 43 - protein, 210 aa, Artificial Sequence
  Escherichia coli K-12 W3110 JW5702 CRP
MVLGKPQTDPTLEWFLSHCHIHKYPSKSKLIHQGEKAETLYYIVKGSVAVLIKDEEGKEMILSYLNQGDFIGELGLFEEGQERSAWVRAKTACEVAEISYKKFRQLIQVNPDILMRLSAQMARRLQVTSEKVGNLAFLDVTGRIAQTLLNLAKQPDAMTHPDGMQIKITRQEIGQIVGCSRETVGRILKMLEDQNLISAHGKTIVVYGTR

SEQ ID NO: 44 - DNA, 633 bp, Artificial Sequence
  Escherichia coli K-12 W3110 JW5702 CRP
atggtgcttggcaaaccgcaaacagacccgactctcgaatggttcttgtctcattgccacattcataagtacccatccaagagcaagcttattcaccagggtgaaaaagcggaaacgctgtactacatcgttaaaggctctgtggcagtgctgatcaaagacgaagagggtaaagaaatgatcctctcctatctgaatcagggtgattttattggcgaactgggcctgtttgaagagggccaggaacgtagcgcatgggtacgtgcgaaaaccgcctgtgaagtggctgaaatttcgtacaaaaaatttcgccaattgattcaggtaaacccggacattctgatgcgtttgtctgcacagatggcgcgtcgtctgcaagtcacttcagagaaagtgggcaacctggcgttcctcgacgtgacgggccgcattgcacagactctgctgaatctggcaaaacaaccagacgctatgactcacccggacggtatgcaaatcaaaattacccgtcaggaaattggtcagattgtcggctgttctcgtgaaaccgtgggacgcattctgaagatgctggaagatcagaacctgatctccgcacacggtaaaaccatcgtcgtttacggcactcgttaa

SEQ ID NO: 45 - protein, 210 aa, Artificial Sequence
  Salmonella enterica subsp. enterica serovar Typhimurium DT104 DT104_34511 CRP
MVLGKPQTDPTLEWFLSHCHIHKYPSKSTLIHQGEKAETLYYIVKGSVAVLIKDEEGKEMILSYLNQGDFIGELGLFEEGQERSAWVRAKTACEVAEISYKKFRQLIQVNPDILMRLSSQMARRLQVTSEKVGNLAFLDVTGRIAQTLLNLAKQPDAMTHPDGMQIKITRQEIGQIVGCSRETVGRILKMLEDQNLISAHGKTIVVYGTR

SEQ ID NO: 46 - DNA, 633 bp, Artificial Sequence
  Salmonella enterica subsp. enterica serovar Typhimurium DT104 DT104_34511 CRP
atggtgcttggcaaaccgcaaacagacccgactcttgaatggttcttgtctcattgccacattcataagtacccgtcaaagagcacgctgattcaccagggtgaaaaagcagaaacgctgtactacatcgttaaaggctccgtggcagtgctgatcaaagatgaagaagggaaagaaatgatcctttcttatctgaatcagggtgattttattggtgaactgggcctgtttgaagaaggccaggaacgcagcgcctgggtacgtgcgaaaaccgcatgtgaggtcgctgaaatttcctacaaaaaatttcgccaattaatccaggtcaacccggatattctgatgcgcctctcttcccagatggctcgtcgcttacaagtcacctctgaaaaagtaggtaacctcgccttccttgacgtcaccgggcgtatcgctcagacgctgctgaatctggcgaaacagcccgatgccatgacgcacccggatgggatgcagatcaaaatcactcgtcaggaaatcggccagatcgtcggctgctcccgcgaaaccgttggtcgtattttgaaaatgctggaagatcaaaacctgatctccgcgcatggcaagaccatcgtcgtctacggcacccgttaa

SEQ ID NO: 47 - protein, 210 aa, Artificial Sequence
  Shigella flexneri 2002017 (serotype Fxv) SFxv_3687 CRP
MVLGKPQTDPTLEWFLSHCHIHKYPSKSTLIHQGEKAETLYYIVKGSVAVLIKDEEGKEMILSYLNQGDFIGELGLFEEGQERSAWVRAKTACEVAEISYKKFRQLIQVNPDILMRLSAQMARRLQVTSEKVGNLAFLDVTGRIAQTLLNLAKQPDAMTHPDGMQIKITRQEIGQIVGCSRETVGRILKMLEDQNLISAHGKTIVVYGTR

SEQ ID NO: 48 - DNA, 633 bp, Artificial Sequence
  Shigella flexneri 2002017 (serotype Fxv) SFxv_3687 CRP
atggtgcttggcaaaccgcaaacagacccgactctcgaatggttcttgtctcattgccacattcataagtacccatccaagagcacgcttattcaccagggtgaaaaagcggaaacgctgtactacatcgttaaaggctctgtggcagtgctgatcaaagacgaagagggtaaagaaatgatcctctcctatctgaatcagggtgattttattggcgaactgggcctgtttgaagagggccaggaacgtagcgcatgggtacgtgcgaaaaccgcctgtgaagtggctgaaatttcgtacaaaaaatttcgccaattgattcaggtaaacccggacattctgatgcgtctgtctgcacagatggcgcgtcgtctgcaagtcacttcagagaaagtgggcaacctggcgttcctcgacgtgacgggccgcattgcacagactctgctgaacctggcaaaacaaccagatgctatgactcacccggacggtatgcaaatcaaaattacccgtcaggaaatcggtcagattgtcggctgttctcgtgaaaccgtgggacgcattctgaagatgctggaagatcagaacctgatctccgcacacggtaaaaccatcgtcgtttacggcactcgttaa

SEQ ID NO: 49 - DNA, 29 bp, Artificial Sequence
  K12 Repeat
cggtttatccccgctggcgcggggaactc

SEQ ID NO: 50 - DNA, 28 bp, Artificial Sequence
  Repeat
ggtttatccccgctggcgcggggaacac

SEQ ID NO: 51 - DNA, 27 bp, Artificial Sequence
  Repeat
cggtttatccccgctggcgcggggaac

SEQ ID NO: 52 - DNA, 29 bp, Artificial Sequence
  O157H7 Repeat
cggtttatccccgctggcgcggggaacac

SEQ ID NO: 53 - DNA, 29 bp, Artificial Sequence
  UK-1 Repeat
cggtttatccccgctggcgcggggaacac

SEQ ID NO: 54 - DNA, 30 bp, Artificial Sequence
  E coli K12 CRISPR I leader sequence
ctaaaagtatacatttgttcttaaagcatt

SEQ ID NO: 55 - DNA, 32 bp, Artificial Sequence
  E coli K12 CRISPR II leader sequence
tctaaacataacctattattaattaatgattt

SEQ ID NO: 56 - protein, 887 aa, Artificial Sequence
  Salmonella enterica subsp. enterica serovar Typhimurium 14028S Cas3
MSIYHYWGKSRRGETDGGDDYHLLCWHSLDVAAVGYWMVINNIYFIDHYLKKLGIQDKEQAAQFFAWILCWHDIGKFAHSFQQLYRHEALNIFNEPTRHYEKIAHTTLGYMLWNSWLSECPELFPPSSLSVRKSKRVMALWMPVTTGHHGRPPEAIQELDHFRQQDKDAARDFLLRIKALFPLITLPEAWDEDEGIDQFQQLSWFISAAVVLADWTGSASRYFPRTAEKMPVDTYWQQALAKAQTAITLFPSAANVSAFTGIETLFPFIQHPTPLQQKALELDINVDGAQLFILEDVTGAGKTEAALILAHRLMAAGKAQGLYFGLPTMATANAMFERMANTWLALYQPDSRPSLILAHSARRLMDRFNQSIWSVTLSGTEEPDEAQPYSQGCAAWFADSNKKALLAEVGVGTLDQAMMAVMPFKHNNLRLLGLSNKILLADEIHACDAWMSRILEGLIERQASNGNATILLSATLSQQQRDKLVAAFSRGVRRSVQAPLLGHDDYPWLTQVTQTELISQRVDTRKEVERCVDIGWLHSEEACLERIGEAVEKGNCIAWIRNSVDDAIRIYRQLQLSKVVVTENLLLFHSRFAFYDRQRIESQTLNLFGKQSGAQRAGKVIIATQVIEQSLDIDCDEMISDLAPVDLLIQRAGRLQRHIRDRNGLVKKSGQDERETPVLRILAPEWDDAPRENWLSSAMRNSAYVYPDHGRMWLTQRILREQGTIRMPQSARLLIESVYGEDVNMPVGFAKTEQLQEGKFYCDRAFAGQMLLNFAPGYCAEISDSLPEKMSTRLAEESVTLWLAKIVDSVVTPYASGEHAWEMSVLRVRQSWWNKHKDEFEKLDGEPLRKWCAQQHQDKDFATVIVVTDFAACGYSANEGLIGMMGE

SEQ ID NO: 57 - DNA, 2664 bp, Artificial Sequence
  Salmonella enterica subsp. enterica serovar Typhimurium 14028S Cas3 nucleotide sequence
gtgtcgatatatcactattggggaaagtctcgacgaggagaaactgacggcggtgatgattaccatttgctttgctggcattctttagatgttgcggctgtgggttactggatggtgataaataatatttattttattgaccactatctaaaaaaattaggcatccaggataaggagcaggcggcgcaattttttgcctggattttatgttggcatgatattggaaagtttgctcattccttccagcaactataccgtcatgaggctttaaatatctttaatgagcctacacggcattatgaaaaaatcgcgcataccacgctgggatacatgttgtggaactcctggctaagtgaatgccctgaattgtttcctccttcttcgctttcagttcgtaaaagtaagcgcgttatggcgctttggatgccagtcactacaggtcatcatggacgccctccagaggcaatccaggagctggaccattttcgccagcaggataaagacgcggcaagagattttcttctgagaataaaagcgctctttcctttaattactttgcctgaagcctgggatgaagatgagggtatcgaccaatttcagcaactttcctggtttatttccgctgcggttgtactggctgactggactggttctgccagccgttattttccgcgtactgcggaaaaaatgcctgttgatacctactggcagcaagctctcgctaaagcacaaactgccatcacgctatttccctcagcggcgaatgtgtctgcctttacgggcatagaaacgcttttcccttttattcagcatcccacaccgttacaacaaaaggcgcttgagctggatatcaacgtggatggcgcccaactctttattcttgaagatgtcaccggggccggaaaaacagaggcggcgctcatattagctcatcgactgatggcggcaggtaaagcgcagggactctattttggactgccgacaatggcgacagccaacgcgatgtttgaacgtatggcgaacacctggctggcgctgtatcagccggactcccgtcccagcctgattctggcgcatagcgcgcgtcgcttaatggatcgtttcaatcagtcaatatggtcggtcactctttctggtacggaagaacccgatgaagcgcagccttatagtcagggatgcgccgcctggtttgccgacagcaataaaaaagcgttgttggcggaggttggcgtaggcacgttggatcaggcgatgatggcggtaatgccatttaaacataacaacctgcggttactgggtcttagcaacaagatcttactggctgatgagatccatgcctgtgatgcctggatgtcccgaatacttgaaggtttgatcgaacggcaggccagtaatggcaacgccactattctgttatctgcgacgctatcgcagcagcagcgagataagctggtggcggcattttcccgtggggtgaggcgtagtgtgcaggcgccgttgctaggccatgacgattatccctggctgactcaggtcacacaaacagagctgatttctcagcgggttgatacacgcaaagaggttgagcgttgcgtagatattggctggctacatagtgaagaggcgtgtcttgaacgtataggtgaagcagtggaaaaaggaaactgtatcgcctggatacgtaactccgttgatgatgcgattcgtatctatcgccagcttcaactgagtaaggtcgtcgtcacggaaaaccttttactcttccatagtcgctttgctttttacgatcgtcagcggattgagtcacagacgctgaatctctttggcaaacagagcggcgcgcaacgtgccggtaaggtcattatcgccacgcaggtcatcgaacaaagtctggatattgactgcgatgagatgatctctgatttagcgccggtggatttattaattcagcgggccggtcgactacagcgtcatattcgcgatcgtaacggtctggtgaaaaagagtgggcaggatgagcgagagacgccagtgctgcgcattcttgctccggagtgggatgacgcgccgcgagagaactggttatccagcgccatgcgtaacagcgcctatgtctatcccgatcatgggcgcatgtggctgacacagcgcatattacgtgagcaggggacgattcggatgccgcaatctgcccgattgttgattgagtcggtctacggcgaggatgtcaacatgccggttggatttgcaaaaaccgagcaattgcaggaaggcaaattttattgcgaccgggcatttgccggccagatgctgcttaactttgcgccgggctactgtgctgaaattagcgattctttaccggagaaaatgtcaacgcggctggcggaagagtctgtcacgctgtggctggcgaaaatcgtggatagcgtcgtaaccccttatgccagcggtgaacacgcctgggagatgagcgtgctgcgagtacgtcagagctggtggaataaacataaagacgagtttgaaaaattagacggcgaacccttgcgtaagtggtgtgcgcaacagcatcaggataaggattttgccacggtgattgtggtgacggactttgccgcttgtggttattcggcgaatgagggattgattggcatgatgggggaataa

SEQ ID NO: 58 - protein, 899 aa, Artificial Sequence
  Escherichia coli Cas3
MRKYPLSLLKDKNIVTFFDFWGKTRRGEKEGGDGYHLLCWHSLDVAAMGYLMVKRNCFGLADYFRQLGISDKEQAAQFFAWLLCWHDIGKFARSFQQLYLAPELKIPEGSRKNYEKISHSTLGYWLWNYYLSECEELLPSSSLSSRKLTRVIEMWMSITTGHHGRPPDRIDELDNFLPEDKAAARDFLLEIKALFPLIEIPTFWDDDEGVELLKQLSWYISATVVLADWTGSSTRFFPRVAHPMDIKDYWQKTLVQAQNALTVFPPKAETAPFTGINTLFPFIEHPTPLQQKVLDLDISQPGPQLFILEDVTGAGKTEAALILAHRLMAARKAQGLFFGLPTMATANAMYDRLVKTWLAFYSPESRPSLVLAHSARTLMDRFNESLWSGDLVGSEEPDEQTFSQGCAAWFANSNKKALLAEIGVGTLDQAMMAVMPFKHNNLRLLGLSNKILLADEIHACDAYMSCILEGLIERQARGGNSVILLSATLSQQQRDKLVAAFARGTEGQQEAPFLEKDDYPWLTHVTKSDVNSHRVATRKDVERSVSVGWLHSEQESIARIESAVSQGKCIAWIRNSVDDAIKVHRQLLARGVIPASSLSLFHSRFAFSDRQRIEMETLARFGKEDGSQRAGKVLICTQVLEQSVDCDLDEMISDLAPVDLLIQRAGRLQRHIRDINGQLKRDGKDERSPPELLILAPVWDDAPGDEWFGSAMRNSAYVYPDHGRIWLTQRVLREQGAIQMPHAARLLIESVYGEDVVMPEGFARSEQEQVGKYYCDRAMAKKFVLNFKPGYAANINDYLPEKLSTRLAEESVSLWLATCIAGVVKPYATGAHAWEMSVVRVRRSWWKKHRDEFSLLEGEAFRQWCIEQRQDPEMANVILVTDDESCGYSAREGLIGKVD

SEQ ID NO: 59 - DNA, 2700 bp, Artificial Sequence
  Escherichia coli Cas3 Strain O157H7 EDL933 (EHEC)
atgcgtaaatatcctttaagtttactgaaggataaaaatattgtgactttctttgatttctggggaaaaacccgacgtggcgagaaagagggtggcgacggctatcaccttctttgctggcattcgctggatgtggccgcaatgggctatttaatggttaaaagaaattgcttcgggctggctgattactttcgtcaattagggatttctgacaaggaacaggcggctcaatttttcgcttggttgctgtgctggcacgatattggaaaatttgcccgctcttttcagcaactttacctggcccctgaactcaagattccggaaggttccagaaagaattacgaaaagatctctcattcaacgctgggttactggctgtggaattattatttaagtgaatgtgaggagttgcttccttcatcttcactctcttctcgtaaacttacacgtgtaatagagatgtggatgtccataactaccgggcatcatggtcgaccacctgaccgtattgatgagctggataattttctgcctgaagacaaagctgccgcgcgagattttctccttgaaatcaaggcactgtttccgctcatagagattcccacattctgggatgatgacgagggcgttgaacttttaaaacaactttcctggtatatctctgcaacagtcgtactcgcagactggacgggttcgtcaacgcgattttttccacgcgtcgcacacccaatggatattaaagattactggcagaaaactttagttcaggctcaaaacgccttaaccgtctttcctccaaaagcagaaaccgcacctttcaccggaattaatacgctgtttccttttattgagcacccgacaccattacagcaaaaggtactggatctggatatcagccagccagggccacagttatttattctggaagacgtgactggcgcaggtaaaacagaagcggcgcttatcctggcgcacaggttgatggctgcgaggaaagcacagggtttgttttttggcctgccaacaatggcaacggccaatgccatgtacgatcggctggtcaaaacctggcttgctttctattcgccagagtcccgccccagcttggtgctggcacacagtgcccgcacattaatggaccgcttcaatgaatcactctggtccggtgatttagtcgggtcagaagaaccggatgaacaaacattcagtcagggatgtgcggcctggtttgccaacagtaacaagaaggcgctactggctgaaattggcgtcggcacgctggatcaggcgatgatggcagtgatgccgtttaaacataataatctgcggcttctggggttgagtaacaaaatcctgctggctgatgagatccatgcctgtgatgcttacatgtcgtgcattcttgaagggctgatcgagcggcaggcgcgtggcggaaacagcgtcattttgctttctgctacgttatcccaacagcagcgcgacaaactcgtcgccgcctttgcgcgtggcacagagggccagcaagaagctccgttccttgaaaaggatgattacccctggctgacgcatgtcacgaaatccgatgtgaactcacaccgggtagcgacgcgcaaagacgttgagcgtagcgtcagcgtgggttggcttcatagtgaacaagagagtattgcgcgtatcgaatcggcggtaagtcagggaaaatgcatcgcctggatccggaattctgtcgatgacgctattaaggttcatcgtcagctgcttgcccgcggcgtcattcccgcttccagcctttcactctttcatagccgctttgcttttagcgatcgccagcgaattgaaatggagacgctggcacgctttggtaaagaagacggttcacagcgtgccggaaaagtcctcatttgtactcaggtcttagagcagagcgttgattgtgacctggacgaaatgatctccgacctggcccctgttgatttgctgattcagcgagcggggcgattacagcggcatatccgcgatattaatggtcagttaaagcgtgacggaaaagacgagcgttcccctcctgaattgctgattctggcccccgtctgggacgacgctcctggtgacgaatggttcggcagtgccatgcgtaacagtgcatatgtctatcccgatcatggacgaatctggctgacgcagcgtgtactgcgtgagcaaggcgctattcaaatgccacacgcagcccgccttcttattgaatcagtctacggtgaggacgtggtaatgccggaaggatttgcccgcagcgagcaggagcaagtgggcaaatattactgcgatcgcgcaatggctaaaaagtttgtcctgaacttcaagcctggctatgccgccaatatcaacgattaccttccggaaaagctgtcgacacgtctggctgaggaatctgtttccctgtggctggctacctgtattgccggtgtggtgaagccttatgccaccggtgctcacgcatgggaaatgagcgttgtcagagtgcgtcgaagctggtggaaaaaacatcgggatgagttttctttactggaaggggaagcgttcaggcagtggtgcattgaacagcggcaagatccggaaatggcaaacgtgattttagtcactgatgacgaaagttgcgggtattcggccagggagggattgattggcaaggttgattga

SEQ ID NO: 60 - protein, 888 aa, Artificial Sequence
  Escherichia coli Cas3 Strain K12
MEPFKYICHYWGKSSKSLTKGNDIHLLIYHCLDVAAVADCWWDQSVVLQNTFCRNEMLSKQRVKAWLLFFIALHDIGKFDIRFQYKSAESWLKLNPATPSLNGPSTQMCRKFNHGAAGLYWFNQDSLSEQSLGDFFSFFDAAPHPYESWFPWVEAVTGHHGFILHSQDQDKSRWEMPASLASYAAQDKQAREEWISVLEALFLTPAGLSINDIPPDCSSLLAGFCSLADWLGSWTTTNTFLFNEDAPSDINALRTYFQDRQQDASRVLELSGLVSNKRCYEGVHALLDNGYQPRQLQVLVDALPVAPGLTVIEAPTGSGKTETALAYAWKLIDQQIADSVIFALPTQATANAMLTRMEASASHLFSSPNLILAHGNSRFNHLFQSIKSRAITEQGQEEAWVQCCQWLSQSNKKVFLGQIGVCTIDQVLISVLPVKHRFIRGLGIGRSVLIVDEVHAYDTYMNGLLEAVLKAQADVGGSVILLSATLPMKQKQKLLDTYGLHTDPVENNSAYPLINWRGVNGAQRFDLLAHPEQLPPRFSIQPEPICLADMLPDLTMLERMIAAANAGAQVCLICNLVDVAQVCYQRLKELNNTQVDIDLFHARFTLNDRREKENRVISNFGKNGKRNVGRILVATQVVEQSLDVDFDWLITQHCPADLLFQRLGRLHRHHRKYRPAGFEIPVATILLPDGEGYGRHEHIYSNVRVMWRTQQHIEELNGASLFFPDAYRQWLDSIYDDAEMDEPEWVGNGMDKFESAECEKRFKARKVLQWAEEYSLQDNDETILAVTRDGEMSLPLLPYVQTSSGKQLLDGQVYEDLSHEQQYEALALNRVNVPFTWKRSFSEVVDEDGLLWLEGKQNLDGWVWQGNSIVITYTGDEGMTRVIPANPK

SEQ ID NO: 61 - DNA, 2667 bp, Artificial Sequence
  Escherichia coli Cas3 Nucleotide sequence
atggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgtgggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataa

SEQ ID NO: 62 - protein, 926 aa, Artificial Sequence
  Streptococcus thermophilus Cas3
MKHINDYFWAKKTEENSRLLWLPLTQHLEDTKNIAGLLWEHWLSEGQKVLIENSINVKSNIENQGKRLAQFLGAVHDIGKATPAFQTQKGYANSVDLDIQLLEKLERAGFSGISSLQLASPKKSHHSIAGQYLLSHYGVDEDIATIIGGHHGRPVDDLDGLNSQKSYPSNYYQDEKKDSLVYQKWKSNQEAFLNWALTETGFNSVSQLPKIKQPAQVILSGLLIMSDWIASNEHFFPLLSLDETDVKNKSQRIETGFKKWKKSNLWQPETFVDLVTLYQERFGFSPRNFQLILSQTIEKTTNPGIVILEAPMGIGKTEAALAVSEQLSSKKGCSGLFFGLPTQATSNGIFKRIEQWTENIKGNNSDHFSIQLVHGKAALNTDFIELLKGNTINMDDSENGSIFVNEWFSGRKTSALDDFVVGTVDQFLMVALKQKHLALRHLGFSKKVIVIDEVHAYDAYMSQYLLEAIRWMGAYGVPVIILSATLPAQQREKLIKSYMAGMGVKWRDIENIDQIKIDAYPLITYNDGPDIHQVKMFEKQEQKNIYIHRLPEEQLFDIVKEGLDNGGVVGIIVNTVRKSQELARNFSDIFGDDMVDLLHSNFIATERIRKEKDLLQEIGKKAIRPPKKIIIGTQVLEQSLDIDFDVLISDLAPMDLLIQRIGRLHRHKIKRPQKHEVARFYVLGTFEEFDFDEGTRLVYGDYLLARTQYFLPDKIRLPDDISPLVQKVYNSDLTITFPKPELHKKYLDAKIEHDDKIKNKETKAKSYRIANPVLKKSRVRTNSLIGWLKNLHPNDSEEKAYAQVRDIEDTVEVIALKKISDGYGLFIENKDISQNITDPIIAKKVAQNTLRLPMSLSKAYNIDQTINELERYNNSHLSQWQNSSWLKGSLGIIFDKNNEFILNGFKLLYDEKYGVTIERLDKNESV

SEQ ID NO: 63 - DNA, 2781 bp, Artificial Sequence
  Streptococcus thermophilus Cas3
atgaaacatattaatgattatttttgggctaagaaaacagaggaaaatagtagacttctttggttaccattaactcaacacttagaagacacgaaaaatattgcaggcctcttatgggaacattggttaagtgaaggacaaaaggtattaattgaaaattctattaatgttaaatcaaatattgaaaaccaagggaaaagattggcacaattcctaggagctgttcatgatatcggtaaagcaacaccagcttttcagacgcaaaaaggttatgcaaattcagtagatttggatattcaattgttagaaaaattggaacgcgcaggtttttctggcattagttctctccaactagcctcccccaaaaagagtcatcatagcattgcaggtcaatatttgttatcccattatggcgtggacgaagatattgcaacaattattggtggacaccatggacgaccagttgatgatttagacggtttaaattctcaaaaaagctatccctccaattattaccaggatgaaaagaaagatagtctcgtttatcagaaatggaagtcaaatcaagaagcttttttaaactgggctttaacagaaacagggtttaattctgtgtctcagcttccaaaaatcaaacagcctgctcaagttattctatcaggtttactcataatgtctgactggattgctagtaatgagcatttttttcctttgttaagtttggatgaaactgatgtgaaaaacaagagtcaacgtattgaaactgggtttaaaaagtggaaaaaatctaacttgtggcaacctgaaactttcgttgaccttgttactctttatcaggaaagatttggatttagtccacgaaattttcagctgatactctcacaaacaatcgaaaagacgactaatcctgggatagtgatactggaagcgccaatgggaatcgggaaaacagaggcggctctagcggtatcagagcagttatctagtaaaaaaggatgtagtggattgttttttggattgcccacacaagcaacctccaatggaatttttaagaggattgaacagtggacagagaatataaagggtaacaattctgatcatttttccattcagctggttcatggaaaagcagccttaaatacggattttattgagttacttaaaggaaatacaattaatatggacgactcggaaaacggcagtatttttgtcaatgagtggttttctgggagaaaaacttcagcattagatgattttgtagttgggacggtcgaccaatttttaatggtggctttaaaacaaaaacatttggccttacgtcatttaggatttagtaaaaaagttatcgttattgatgaagtccacgcttatgatgcttatatgagccaatatttgttggaagctatcagatggatgggagcttatggtgttcctgtaattattttatcagcaactttacctgcccaacaaagagaaaaactcataaaaagctatatggctggaatgggagtgaaatggcgagatattgaaaatatagatcagataaaaatagacgcataccctttaatcacttataatgacgggcctgacattcatcaagttaaaatgttcgaaaagcaagaacaaaaaaatatctacattcatcgtttaccagaagaacagttatttgatattgtaaaagaaggtcttgacaatggtggagtagttgggataattgtcaatacggtgagaaaatctcaagaattggcaagaaatttttcagatatttttggagatgatatggtagatttgcttcattctaatttcatagcaactgaaagaatccgaaaagaaaaggatttattgcaagaaattgggaaaaaagcaatacgtccaccaaagaaaatcattattggtacacaggtgcttgaacagtcgttagatattgattttgatgtactgataagcgacttagcgcctatggatttactcattcaacgtatcggacgactacatcgtcacaaaatcaaaaggccccaaaagcacgaagtagcaagattttatgttttaggaacatttgaagagtttgattttgatgaaggaacgcgtttggtttatggggactacctattagctagaactcagtactttttaccagataaaatacgacttcctgatgatatttcaccgctagtccaaaaggtttataattcagacctaacaattacgtttccaaagccagaacttcataaaaaatatttggatgctaaaatagaacatgatgataagattaaaaataaagaaacaaaggcaaagtcataccgtattgctaatcctgtcttaaaaaaatcgagagttcgaactaacagtttgattggttggttaaagaacctccatccaaatgatagtgaagaaaaagcatatgctcaagttcgagatattgaagatacagttgaagtgattgcattaaaaaaaatatctgatgggtatggtttgttcatagaaaataaagatatatctcagaacattactgatcctataattgcaaaaaaggtagcacaaaatactttacgacttccgatgagtttatccaaagcctataatattgatcaaacgattaatgagcttgaaagatataacaatagccacttaagtcaatggcaaaactcatcatggttaaagggatctcttgggattatttttgataaaaacaatgagtttatactgaatggatttaaactattatatgatgaaaaatatggtgttaccatagaaaggttggataagaatgagtcggtttaa

SEQ ID NO: 64 - protein, 887 aa, Artificial Sequence; identical to SEQ ID NO: 56
  Salmonella enterica subsp. enterica serovar Typhimurium LT2 Cas 3

SEQ ID NO: 65 - DNA, 2664 bp, Artificial Sequence; identical to SEQ ID NO: 57
  Salmonella enterica subsp. enterica serovar Typhimurium LT2 Cas 3 nucleotide sequence

SEQ ID NO: 66 - protein, 502 aa, Artificial Sequence
  Escherichia coli K-12 MG1655 b2760 CasA
MNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 67 - DNA, 1509 bp, Artificial Sequence
  Escherichia coli K-12 MG1655 b2760 CasA
atgaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 68 - protein, 520 aa, Artificial Sequence
  Escherichia coli O157 H7 EC4115 (EHEC) ECH74115_4013 Cse1
MNSFSLLTTPWLPVRFKDGTTGKLAPVDLADENVVDIAAPRADLQGAAWQFLLGLLQSSFAPKDYRRWDDIWEDGLEAEKLREALLSLEHPFQFGPDSPSFMQDFEVLMGDKVQVASLLPEIPGAQTTKFNKDHFIKRGVTEHVCSHCSALALFSLQLNAPSGGKGYRTGLRGGGPMTTLIELQEYQGNQQAPLWRKLWLNVMPQDEADLPLPKKFDDLVFPWLGPTRTSELAGAVVTDDQVNKLQAYWGMPRRIRIDFNTTTVGNCDICGEQSDALLSLMTTKNYGANYAMWQHPLTPYRVPLKEGGEFYSVKPQPGGLIWRDWLGLIETGKSENNTELPALVVKLFNASSLKQAKVGLWGFGYDFDNMKARCWYEHHFPLLLNKKEGQIPKLRLAAQTASRILSLLRSALKEAWFSDPKGARGDFSFVDIDFWNKTQHRFLRLVRQIEEGQDADELLGKWQKEIWLFARQDFDERVFTNPYEPVDLERVMTARKKYFTTSAEKQSAKAAREKKQEAAE

SEQ ID NO: 69 - DNA, 1563 bp, Artificial Sequence
  Escherichia coli O157 H7 EC4115 (EHEC) ECH74115_4013 Cse1
atgaactcgttttcacttctgacaaccccgtggttgcccgttcgttttaaagacggaacaacaggcaagctggcgccagtcgatctggcggatgaaaatgttgtcgatatcgctgcgccgcgggcagatctccagggggcggcatggcagtttttgctggggttactacaaagcagtttcgcgccaaaagattatcgtcgttgggatgatatctgggaagacgggctggaagctgaaaagctacgggaagcattgctgtcattagaacaccctttccagtttggcccagattcaccttcatttatgcaggatttcgaggtgctcatgggcgataaagttcaggtcgcttcgctactgcctgagattcccggcgctcaaacaacgaagtttaataaagaccactttattaagcgtggcgtgactgaacacgtatgctctcattgttctgcgttagctctgttctccctacagttaaatgcgccgtcaggtggcaaaggctatcgcaccggtttacgcggcggtgggccgatgacgactctgattgaattgcaggagtatcagggcaatcaacaagcccccttgtggcgcaaactgtggctcaacgtgatgccgcaggatgaagccgacttaccgctacccaaaaaatttgacgatctggttttcccctggcttggcccgacgcgtaccagcgaactggccggtgcggtggtaaccgatgatcaggtcaataaactccaggcgtactggggaatgccgcggcgtattcgtattgattttaataccacgacagtcggcaactgcgatatttgcggtgagcagagtgacgcgcttctgagtttgatgactaccaaaaattacggtgcgaattatgccatgtggcagcatcccttaacgccttaccgtgtaccacttaaagagggcggtgagttttactccgttaaaccacaaccgggcggtttaatctggcgcgactggttaggccttatcgaaacgggtaagtcagaaaacaatacggaacttcccgcgctggtggtgaaactctttaatgccagcagtctgaaacaggcaaaagtgggcctgtggggatttggttatgatttcgacaacatgaaagcgcgctgttggtacgaacaccatttcccgctgctgctcaataaaaaagaaggccagataccgaagctgcggctggctgcgcaaacggcttcacggattctgagtctgttacggagtgcattgaaagaagcatggttctccgatccaaaaggtgcaaggggtgatttcagttttgtggatatcgacttctggaacaaaactcagcatcgcttcctgaggttagtgcgccaaattgaagaaggtcaggatgcggatgaattactcggcaaatggcaaaaggaaatttggttattcgcacgtcaggattttgacgagcgtgtattcaccaatccttatgagcccgttgatttggaacgcgtcatgaccgcgcgcaagaaatattttacaacatcggcggagaagcaaagtgctaaagccgccagggagaaaaagcaggaggctgctgaatga

SEQ ID NO: 70 - protein, 518 aa, Artificial Sequence
  Salmonella enterica subsp. enterica serovar Typhimurium var. 5-CFSAN001921 CFSAN001921_02360 CasA
MDNFSLLTTPWLPVRFKDGSTGKLAPVDLADENVVDIAATRADLQGAAWQFLLGLLQCSIAPKRYKNWEDIWFDGLHADVLHKALAPLEHAFQFGAETPSFMQDFEPLSGEKVSIASLLPEIPGAQTTKFNKDHFVKRGVTERFCPHCAALALFSLQLNAPAGGKGYRTGLRGGGPLTTLVELQEYQGERQTPLWRKLWLNVMPQDTADLPLPDQCDATVFPWLAATRTSEQANAVTTPEQVNKLQAYWGMPRRIRLDFATLQSGCCDICGAESDELLGFMTVKNYGVNYDGWRHPLTPYRAPVKDQNAFFSVKPQPGGLIWRDWLGLSQNNQTEANYESPAQVVKVFNARSLTDVKAGIWGFGADFDNMKIRCWYEHHFPLLMTEGLIPDLRKAVQTAARLLSLLRSALKEAWFADAKGARGDFSFIDIDFWNLTQGRFLNLIHDLENGHKPDERLNKWQRELWLFTRHYFDDHVFTNPYESSDLERIMTARKKYFTTSAEKQSAKAAKAKKQEAAE

SEQ ID NO: 71 - DNA, 1557 bp, Artificial Sequence
  Salmonella enterica subsp. enterica serovar Typhimurium var. 5-CFSAN001921 CFSAN001921_02360 CasA
atggacaatttttcacttttaacaacgccctggctccccgtccgtttcaaagacggttccacgggcaagctggcccccgtcgatctggcggatgaaaacgtggtggacatcgccgcaacgcgagcagatttacagggagcggcttggcagtttctgttgggattgctgcaatgcagtatcgcgccgaaaagatacaaaaattgggaggatatctggtttgatggattgcatgccgatgtgctccataaggcattagcaccgttagaacacgcttttcagtttggcgcggaaacgccgtcttttatgcaggattttgaaccgttaagcggcgaaaaagtctctattgcctcattgttgccggaaatacctggcgcgcaaaccacgaagttcaataaagatcattttgtcaaacgcggcgtaacggaacgtttttgtccgcactgcgcggcgctggcgctgttctcgttgcagcttaacgcgcctgcgggcggcaaaggctatcgtaccgggctgcgcggcggcgggccactgaccacgctggttgaattgcaggaatatcagggcgagcggcaaacgccgctctggcgcaagctgtggctcaacgtgatgccgcaggatactgcggatctgcctttaccagaccagtgtgatgcgaccgttttcccgtggcttgccgcgacgcggaccagcgagcaggcgaatgccgttaccacgccggagcaggtcaataaactccaggcgtactgggggatgccgcgtcgtatccgcctggattttgccaccttacagtcaggttgctgcgatatttgcggcgctgaaagcgatgagcttcttggctttatgaccgtcaagaactacggcgttaactacgatggctggcggcacccgctgacgccttatcgcgccccggtaaaagatcaaaacgccttcttttccgttaaaccgcagcccggcggccttatctggcgcgactggctgggattaagtcagaacaaccagacggaagcgaattacgaatctcccgcgcaggtagtcaaggtgtttaacgcccgctcgctgactgacgttaaagcggggatctggggctttggcgcggatttcgacaatatgaaaatccgctgctggtatgagcatcacttcccgttgctgatgacggaaggtctgatccctgatttacgtaaggccgtgcaaactgcggcccgcctgttgagcctgcttcgcagcgcgctcaaagaggcctggtttgccgatgcgaagggtgctcgcggtgatttcagttttatcgacattgatttctggaacctgacgcagggacgttttctcaacctgattcacgatctggaaaacggccacaagccggacgaaaggctgaataaatggcaaagagaactttggctgtttacccgtcattacttcgatgatcacgtctttaccaacccctacgagagcagcgatctggaacgcatcatgaccgcgcgcaagaaatattttacgacatcggcggaaaaacaaagtgcaaaagccgccaaagcaaagaaacaggaggctgctgaatga

SEQ ID NO: 72 - protein, 518 aa, Artificial Sequence
  Salmonella enterica subsp. enterica serovar Enteritidis EC20090193 AU37_14140 CasA
MDNFSLLTTPWLPVRFKDGSTGKLAPVDLADENVVDIAATRADLQGAAWQFLLGLLQCSIAPKRYKNWEDIWFDGLHADVLHKALAPLEHAFQFGAESPSFMQDFEPLSGEKVSIASLLPEIPGAQTTKFNKDHFVKRGVTERFCPHCAALALFSLQLNAPAGGKGYRTGLRGGGPLTTLVELQEYQGERQTPIWRKLWLNVMPQDTADLPLPDQCDATVFPWLAATRTSEQANAVTTPEQVNKLQAYWGMPRRIRLDFATLQSGCCDICGAESDELLGFMTVKNYGVNYDGWRHPLTPYRAPVKDQNAFFSVKPQPGGLIWRDWLGLSQNNQTEANYESPAQVVKVFNARSLTDVKAGIRGFGADFDNMKIRCWYEHHFPLLMTEGLIPDLRKAVQTAARLLSLLRSALKEAWFTNAKDARGDFSFIDIDFWNLTQGRFLNLIHDLENGHKPDERLNKWQRELWLFTRCYFDDHVFTNPYESSDLERIMKARKKYFTSSAEKQSAKAAKAKKQEAAE

SEQ ID NO: 73 - DNA, 1557 bp, Artificial Sequence
  Salmonella enterica subsp. enterica serovar Enteritidis EC20090193 AU37_14140 CasA
atggacaatttttcacttttaacaacgccctggctccccgtccgtttcaaagacggttccacgggcaagctggcccccgtcgatctggcggatgaaaacgtggtggacatcgccgcaacgcgagcagatttacagggagcggcctggcagtttctgttgggattgctgcaatgcagtatcgcgccgaaaagatacaaaaattgggaggatatctggtttgatggattgcatgccgatgtgctccataaggcattagcaccgttagaacacgcttttcagtttggcgcggaatccccctcgtttatgcaggattttgaaccgttaagcggcgaaaaagtctctattgcctcattgttgccggaaatacctggcgcgcaaaccacgaagttcaataaagatcattttgtcaaacgcggcgtaacggaacgtttttgtccgcactgcgcggcgctggcgctgttctcgttgcagcttaacgcgcctgcgggcggcaaaggctatcgtaccgggctgcgcggcggcgggccactgaccacgctggttgaattgcaggaatatcagggcgagcggcaaacgccgatctggcgcaagctgtggctcaacgtgatgccgcaggatactgcggatctgcctttaccagaccagtgtgatgcgaccgttttcccgtggcttgccgcgacgcggaccagcgagcaggcgaatgccgttaccacgccggagcaggtcaataaactccaggcgtactgggggatgccgcgtcgtatccgcctggattttgccaccttacagtcaggttgctgcgatatttgcggcgctgaaagcgatgagcttcttggctttatgaccgtcaagaactacggcgttaactacgatggctggcggcacccgctgacgccttatcgcgccccggtaaaagatcaaaacgccttcttttccgttaaaccgcagcccggcggccttatctggcgcgactggctgggattaagtcagaacaaccagacggaagcgaattacgaatctcccgcgcaggtagtcaaggtgtttaacgcccgctcgctgactgacgttaaagcggggatccggggctttggcgcggatttcgacaatatgaaaatccgctgctggtatgagcatcacttcccgttgctgatgacggaaggtctgatccctgatttacgtaaggccgtgcaaactgcggcccgcctgttgagcctgcttcgcagtgcgctaaaagaagcgtggttcaccaatgcgaaggatgcgcggggtgatttcagttttatcgacattgatttctggaacctgacgcaggggcgctttctcaatctgatccacgatctggaaaacggacacaagccggacgaaaggctgaataaatggcaaagagaactttggctgtttacccgttgttacttcgatgatcacgtctttaccaacccctacgagagcagcgatctggagcgcatcatgaaggcgcgcaaaaaatattttacttcatcggcggaaaagcaaagcgcaaaagccgccaaagcaaagaaacaggaggctgctgaatga

SEQ ID NO: 74 - DNA, 28 bp, Artificial Sequence
  S thermophilus CRISPR4 repeat
gtttttcccgcacacgcgggggtgatcc